(19) United States
(12) Patent Application Publication
(10) Pub. No.: US 2019/0367519 A1
(43) Pub. Date: Dec. 5, 2019

(54) SOLVATED FORMS OF A BRUTON'S TYROSINE KINASE INHIBITOR

(71) Applicant: Pharmacyclics LLC, Sunnyvale, CA (US)

(72) Inventors: Erick Goldman, Concord, CA (US); Mark S. Smyth, Granite Bay, CA (US); Thierry Bonnaud, Cambridge, MA (US); Alberto Munoz Garcia, Cambridge (GB); Christopher P. Worrall, Cambridge (GB)

(21) Appl. No.: 16/224,565

(22) Filed: Dec. 18, 2018

Related U.S. Application Data
(63) Continuation of application No. 15/561,615, filed on Sep. 26, 2017, now abandoned.
(60) Provisional application No. 62/139,594, filed on Mar. 27, 2015.

(51) Int. Cl. C07D 487/04; A61K 31/519

(52) U.S. Cl. CPC C07D 487/04; G01N 23/20075; A61K 31/519

(57) ABSTRACT

Described herein are solvates of the Bruton's tyrosine kinase (Btk) inhibitor 1-((R)-3-(4-amino-3-(4-phenoxyphenyl)-1H-pyrazolo[3,4-d]pyrimidin-1-yl)piperidin-1-yl)prop-2-en-1-one, including crystalline forms, and pharmaceutically acceptable salts thereof. Also disclosed are pharmaceutical compositions that include the solvates, as well as methods of using the solvates, alone or in combination with other therapeutic agents, for the treatment of autoimmune diseases or conditions, heteroimmune diseases or conditions, cancer, including lymphoma, and inflammatory diseases or conditions.

### CROSS-REFERENCE TO RELATED APPLICATION

This application claims the benefit of U.S. Provisional Application No. 62/139,594, filed Mar. 27, 2015, which is incorporated herein by reference in its entirety.

### FIELD OF THE INVENTION

Described herein are solvates of the Bruton's tyrosine kinase (Btk) inhibitor 1-((R)-3-(4-amino-3-(4-phenoxyphenyl)-1H-pyrazolo[3,4-d]pyrimidin-1-yl)piperidin-1-yl)prop-2-en-1-one, including crystalline forms and pharmaceutically acceptable salts thereof, as well as pharmaceutical compositions that include the Btk inhibitor and methods of using the Btk inhibitor in the treatment of diseases or conditions that would benefit from inhibition of Btk activity.

### BACKGROUND OF THE INVENTION

Bruton's tyrosine kinase (Btk), a member of the Tec family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses.

Btk is a key regulator of B-cell development, activation, signaling, and survival. In addition, Btk plays a role in a number of other hematopoetic cell signaling pathways, e.g., Toll like receptor (TLR) and cytokine receptor-mediated TNF-α production in macrophages, IgE receptor (FcepsilonRI) signaling in Mast cells, inhibition of Fas/APO-1 apoptotic signaling in B-lineage lymphoid cells, and collagen-stimulated platelet aggregation.

1-((R)-3-(4-amino-3-(4-phenoxyphenyl)-1H-pyrazolo[3,4-d]pyrimidin-1-yl)piperidin-1-yl)prop-2-en-1-one is also known by its IUPAC name as 1-{(3R)-3-[4-amino-3-(4-phenoxyphenyl)-1H-pyrazolo[3,4-d]pyrimidin-1-yl]piperidin-1-yl}prop-2-en-1-one or 2-Propen-1-one, 1-[(3R)-3-[4-amino-3-(4-phenoxyphenyl)-1H-pyrazolo[3,4-d]pyrimidin-1-yl]-1-piperidinyl-, and has been given the USAN name, ibrutinib. The various names given for ibrutinib are used interchangeably herein.

### SUMMARY OF THE INVENTION

Described herein are solvates of the Btk inhibitor 1-((R)-3-(4-amino-3-(4-phenoxyphenyl)-1H-pyrazolo[3,4-d]pyrimidin-1-yl)piperidin-1-yl)prop-2-en-1-one, including pharmaceutically acceptable polymorphs and amorphous phases, and methods of uses thereof. Also described are pharmaceutically acceptable salts of the solvated Btk inhibitor, including pharmaceutically acceptable polymorphs, and amorphous phases, and methods of uses thereof. 1-((R)-3-(4-Amino-3-(4-phenoxyphenyl)-1H-pyrazolo[3,4-d]pyrimidin-1-yl)piperidin-1-yl)prop-2-en-1-one, as well as the pharmaceutically acceptable salts thereof, are used in the manufacture of medicaments for the treatment of diseases or conditions that are associated with Btk activity. 1-((R)-3-(4-Amino-3-(4-phenoxyphenyl)-1H-pyrazolo[3,4-d]pyrimidin-1-yl)piperidin-1-yl)prop-2-en-1-one is an irreversible Btk inhibitor.

Also described herein are methods for preparing crystalline, solvated forms of 1-((R)-3-(4-amino-3-(4-phenoxyphenyl)-1H-pyrazolo[3,4-d]pyrimidin-1-yl)piperidin-1-yl)prop-2-en-1-one. Further described are pharmaceutical compositions that include the solvated Btk inhibitor and methods of using the solvated Btk inhibitor in the treatment of diseases or conditions (including diseases or conditions wherein irreversible inhibition of Btk provides therapeutic benefit to a mammal having the disease or condition).

In one aspect is a solvate of 1-((R)-3-(4-amino-3-(4-phenoxyphenyl)-1H-pyrazolo[3,4-d]pyrimidin-1-yl)piperidin-1-yl)prop-2-en-1-one.

In one embodiment is a solvate, wherein 1-((R)-3-(4-amino-3-(4-phenoxyphenyl)-1H-pyrazolo[3,4-d]pyrimidin-1-yl)piperidin-1-yl)prop-2-en-1-one is solvated with butyronitrile, 1,2-dimethoxyethane, hexafluorobenzene, acetophenone, chlorobenzene, dimethylacetamide, benzyl acetate, or 1,1,2-trichloroethane, or a combination thereof. In one embodiment is a solvate, wherein 1-((R)-3-(4-amino-3-(4-phenoxyphenyl)-1H-pyrazolo[3,4-d]pyrimidin-1-yl)piperidin-1-yl)prop-2-en-1-one is solvated with butyronitrile. In one embodiment is a solvate, wherein 1-((R)-3-(4-amino-3-(4-phenoxyphenyl)-1H-pyrazolo[3,4-d]pyrimidin-1-yl)piperidin-1-yl)prop-2-en-1-one is solvated with 1,2-dimethoxyethane. In one embodiment is a solvate, wherein 1-((R)-3-(4-amino-3-(4-phenoxyphenyl)-1H-pyrazolo[3,4-d]pyrimidin-1-yl)piperidin-1-yl)prop-2-en-1-one is solvated with hexafluorobenzene. In one embodiment is a solvate, wherein 1-((R)-3-(4-amino-3-(4-phenoxyphenyl)-1H-pyrazolo[3,4-d]pyrimidin-1-yl)piperidin-1-yl)prop-2-en-1-one is solvated with acetophenone. In one embodiment is a solvate, wherein 1-((R)-3-(4-amino-3-(4-phenoxyphenyl)-1H-pyrazolo[3,4-d]pyrimidin-1-yl)piperidin-1-yl)prop-2-en-1-one is solvated with chlorobenzene. In one embodiment is a solvate, wherein 1-((R)-3-(4-amino-3-(4-phenoxyphenyl)-1H-pyrazolo[3,4-d]pyrimidin-1-yl)piperidin-1-yl)prop-2-en-1-one is solvated with dimethylacetamide. In one embodiment is a solvate, wherein 1-((R)-3-(4-amino-3-(4-phenoxyphenyl)-1H-pyrazolo[3,4-d]pyrimidin-1-yl)piperidin-1-yl)prop-2-en-1-one is solvated with benzyl acetate. In one embodiment is a solvate, wherein 1-((R)-3-(4-amino-3-(4-phenoxyphenyl)-1H-pyrazolo[3,4-d]pyrimidin-1-yl)piperidin-1-yl)prop-2-en-1-one is solvated with 1,1,2-trichloroethane.

In a further embodiment, the solvate is anhydrous.

In another embodiment the solvate is crystalline.

In yet another embodiment the solvate is amorphous.

In one aspect, described herein is a bis-butyronitrile solvate of 1-((R)-3-(4-amino-3-(4-phenoxyphenyl)-1H-pyrazolo[3,4-d]pyrimidin-1-yl)piperidin-1-yl)prop-2-en-1-one. In one aspect, described herein is a crystalline form (Form 1) of a butyronitrile solvate of 1-((R)-3-(4-amino-3-(4-phenoxyphenyl)-1H-pyrazolo[3,4-d]pyrimidin-1-yl)piperidin-1-yl)prop-2-en-1-one that has at least one of the following properties:
(a) an X-ray powder diffraction (XRPD) pattern substantially the same as shown in FIG. 1;
(b) an X-ray powder diffraction (XRPD) pattern with at least two, four, six, or all of the characteristic peaks at 5.5±0.1° 2-Theta, 10.9±0.1° 2-Theta, 13.6±0.1° 2-Theta, 14.8±0.1° 2-Theta, 17.3±0.1° 2-Theta, 18.7±0.1° 2-Theta, 20.0±0.1° 2-Theta, and 21.8±0.1° 2-Theta;
(c) a DSC thermogram substantially the same as the one set forth in FIG. 2;
(d) a DSC thermogram with an endotherm event at between about 100-125° C.;
(e) a thermo-gravimetric analysis (TGA) thermogram substantially the same as the one set forth in FIG. 2;
or
(f) combinations thereof.

In some embodiments, the crystalline form of the butyronitrile solvate of Compound 1 (Form 1) has an X-Ray powder diffraction (XRPD) pattern substantially the same as shown in FIG. 1. In some embodiments, the crystalline form of the butyronitrile solvate of Compound 1 (Form 1) has an X-ray powder diffraction (XRPD) pattern with characteristic peaks at 5.5±0.1° 2-Theta, 10.9±0.1° 2-Theta, 13.6±0.1° 2-Theta, 14.8±0.1° 2-Theta, 17.3±0.1° 2-Theta, 18.7±0.1° 2-Theta, 20.0±0.1° 2-Theta, and 21.8±0.1° 2-Theta. In some embodiments, the crystalline form of the butyronitrile solvate of Compound 1 (Form 1) has a DSC thermogram substantially the same as the one set forth in FIG. 2. In some embodiments, the crystalline form of the butyronitrile solvate of Compound 1 (Form 1) has a thermo-gravimetric analysis (TGA) thermogram substantially the same as the one set forth in FIG. 2. In some embodiments, the crystalline form of the butyronitrile solvate of Compound 1 (Form 1) has a DSC thermogram with an endotherm at between about 100-125° C. In some embodiments, the endotherm event have an onset at about 110° C., a first peak at about 120° C. and a second peak at about 121° C. In some embodiments, the DSC thermogram further has an endotherm having an onset at about 153° C. and a peak at about 156° C. In some embodiments, the crystalline form of the butyronitrile solvate of Compound 1 (Form 1) is characterized as having properties (a), (b), (c), (d), and (e).

In one aspect, described herein is a hemi-dimethoxyethane of 1-((R)-3-(4-amino-3-(4-phenoxyphenyl)-1H-pyrazolo[3,4-d]pyrimidin-1-yl)piperidin-1-yl)prop-2-en-1-one. In another aspect, described herein is a crystalline form (Form 2) of a 1,2-dimethoxyethane solvate of 1-((R)-3-(4-amino-3-(4-phenoxyphenyl)-1H-pyrazolo[3,4-d]pyrimidin-1-yl)piperidin-1-yl)prop-2-en-1-one that has at least one of the following properties:
(a) an X-ray powder diffraction (XRPD) pattern substantially the same as shown in FIG. 3;
(b) an X-ray powder diffraction (XRPD) pattern with at least two, four, six, or all of the characteristic peaks at 6.8±0.1° 2-Theta, 13.4±0.1° 2-Theta, 17.6±0.1° 2-Theta, 18.2±0.1° 2-Theta, 20.2±0.1° 2-Theta, 21.2±0.1° 2-Theta, and 22.2±0.1° 2-Theta;
(c) substantially the same X-ray powder diffraction (XRPD) pattern post storage at 40° C. and 75% RH for at least a week;
(d) a DSC thermogram substantially the same as the one set forth in FIG. 4;
(e) a DSC thermogram with an endotherm having an onset at about 89° C. and a peak at about 101° C.;
(f) a thermo-gravimetric analysis (TGA) thermogram substantially the same as the one set forth in FIG. 4;
or
(g) combinations thereof.

In some embodiments, the crystalline form of the 1,2-dimethoxyethane solvate of Compound 1 (Form 2) has an X-ray powder diffraction (XRPD) pattern substantially the same as shown in FIG. 3. In some embodiments, the crystalline form of the 1,2-dimethoxyethane solvate of Compound 1 (Form 2) has an X-ray powder diffraction (XRPD) pattern with characteristic peaks at 6.8±0.1° 2-Theta, 13.4±0.1° 2-Theta, 17.6±0.1° 2-Theta, 18.2±0.1° 2-Theta, 20.2±0.1° 2-Theta, 21.2±0.1° 2-Theta, and 22.2±0.1° 2-Theta. In some embodiments, the crystalline form of the 1,2-dimethoxyethane solvate of Compound 1 (Form 2) has a substantially the same X-ray powder diffraction (XRPD) pattern post storage at 40° C. and 75% RH for at least a week. In some embodiments, the crystalline form of the 1,2-dimethoxyethane solvate of Compound 1 (Form 2) has a DSC thermogram substantially the same as the one set forth in FIG. 4. In some embodiments, the crystalline form of the 1,2-dimethoxyethane solvate of Compound 1 (Form 2) has a DSC thermogram with an endotherm having an onset at about 89° C. and a peak at about 101° C. In some embodiments, the crystalline form of the 1,2-dimethoxyethane solvate of Compound 1 (Form 2) has a thermo-gravimetric analysis (TGA) thermogram substantially the same as the one set forth in FIG. 4. In some embodiments, the crystalline form of the 1,2-dimethoxyethane solvate of Compound 1 (Form 2) is characterized as having properties (a), (b), (c), (d), (e), and (f).

In one aspect, described herein is a hexafluorobenzene solvate of 1-((R)-3-(4-amino-3-(4-phenoxyphenyl)-1H-pyrazolo[3,4-d]pyrimidin-1-yl)piperidin-1-yl)prop-2-en-1-one. In another aspect, described herein is a crystalline form (Form 3) of a hexafluorobenzene solvate of 1-((R)-3-(4-amino-3-(4-phenoxyphenyl)-1H-pyrazolo[3,4-d]pyrimidin-1-yl)piperidin-1-yl)prop-2-en-1-one that has at least one of the following properties:
(a) an X-ray powder diffraction (XRPD) pattern substantially the same as shown in FIG. 5;
(b) an X-ray powder diffraction (XRPD) pattern with at least two, four, six, or all of the characteristic peaks at 5.4±0.1° 2-Theta, 14.0±0.1° 2-Theta, 16.1±0.1° 2-Theta, 18.6±0.1° 2-Theta, 19.3±0.1° 2-Theta, 22.4±0.1° 2-Theta, and 23.6±0.1° 2-Theta;
(c) a DSC thermogram substantially the same as the one set forth in FIG. 6;
(d) a DSC thermogram with an endotherm having an onset at about 51° C.;
or
(e) combinations thereof.

In some embodiments, the crystalline form of the hexafluorobenzene solvate of Compound 1 (Form 3) has an X-ray powder diffraction (XRPD) pattern substantially the same as shown in FIG. 5. In some embodiments, the crystalline form of the hexafluorobenzene solvate of Compound 1 (Form 3) has an X-ray powder diffraction (XRPD) pattern with characteristic peaks at 5.4±0.1° 2-Theta, 14.0±0.1° 2-Theta, 16.1±0.1° 2-Theta, 18.6±0.1° 2-Theta, 19.3±0.1° 2-Theta, 22.4±0.1° 2-Theta, and 23.6±0.1° 2-Theta. In some embodiments, the crystalline form of the hexafluorobenzene solvate of Compound 1 (Form 3) has a DSC thermogram substantially the same as the one set forth in FIG. 6. In some embodiments, the crystalline form of the hexafluorobenzene solvate of Compound 1 (Form 3) has a DSC thermogram with an endotherm having an onset at about 51° C. In some embodiments, the endotherm has a peak at about 75° C. In some embodiments, the crystalline form of the hexafluorobenzene solvate of Compound 1 (Form 3) is characterized as having properties (a), (b), (c), and (d).

In another aspect, described herein is a crystalline form (Form 4) of a hexafluorobenzene solvate of 1-((R)-3-(4-amino-3-(4-phenoxyphenyl)-1H-pyrazolo[3,4-d]pyrimidin-1-yl)piperidin-1-yl)prop-2-en-1-one that has at least one of the following properties:
(a) an X-ray powder diffraction (XRPD) pattern substantially the same as shown in FIG. 7;
(b) an X-ray powder diffraction (XRPD) pattern with at least two, four, or all of the characteristic peaks at 12.6±0.1° 2-Theta, 15.4±0.1° 2-Theta, 17.7±0.1° 2-Theta, 24.9±0.1° 2-Theta, 25.4±0.1° 2-Theta, and 26.9±0.1° 2-Theta;
(c) a DSC thermogram substantially the same as the one set forth in FIG. 8;
(d) a DSC thermogram with an endotherm having an onset at about 84° C. and a peak at about 100° C.;
(e) a thermo-gravimetric analysis (TGA) thermogram substantially the same as the one set forth in FIG. 8;
or
(f) combinations thereof.

In some embodiments, the crystalline form of the hexafluorobenzene solvate of Compound 1 (Form 4) has an X-ray powder diffraction (XRPD) pattern substantially the same as shown in FIG. 7. In some embodiments, the crystalline form of the hexafluorobenzene solvate of Compound 1 (Form 4) has an X-ray powder diffraction (XRPD) pattern with characteristic peaks at 12.6±0.1° 2-Theta, 15.4±0.1° 2-Theta, 17.7±0.1° 2-Theta, 24.9±0.1° 2-Theta, 25.4±0.1° 2-Theta, and 26.9±0.1° 2-Theta. In some embodiments, the crystalline form of the hexafluorobenzene solvate of Compound 1 (Form 4) has a DSC thermogram substantially the same as the one set forth in FIG. 8. In some embodiments, the crystalline form of the hexafluorobenzene solvate of Compound 1 (Form 4) has a DSC thermogram with an endotherm having an onset at about 84° C. and a peak at about 100° C. In some embodiments, the crystalline form of the hexafluorobenzene solvate of Compound 1 (Form 4) has a thermo-gravimetric analysis (TGA) thermogram substantially the same as the one set forth in FIG. 8. In some embodiments, the crystalline form of the hexafluorobenzene solvate of Compound 1 (Form 4) is characterized as having properties (a), (b), (c), (d), and (e).

In another aspect, described herein is an acetophenone solvate of 1-((R)-3-(4-amino-3-(4-phenoxyphenyl)-1H-pyrazolo[3,4-d]pyrimidin-1-yl)piperidin-1-yl)prop-2-en-1-one. In another aspect, described herein is a crystalline form (Form 5) of an acetophenone solvate of 1-((R)-3-(4-amino-3-(4-phenoxyphenyl)-1H-pyrazolo[3,4-d]pyrimidin-1-yl)piperidin-1-yl)prop-2-en-1-one that has at least one of the following properties:
(a) an X-ray powder diffraction (XRPD) pattern substantially the same as shown in FIG. 9;
(b) an X-ray powder diffraction (XRPD) pattern with at least two, four, six or all of the characteristic peaks at 7.6±0.1° 2-Theta, 8.8±0.1° 2-Theta, 15.2±0.1° 2-Theta, 17.6±0.1° 2-Theta, 18.9±0.1° 2-Theta, 19.5±0.1° 2-Theta, 20.4±0.1° 2-Theta, 21.0±0.1° 2-Theta, 21.3±0.1° 2-Theta, 21.8±0.1° 2-Theta, 24.3±0.1° 2-Theta, and 24.8±0.1° 2-Theta;
(c) a DSC thermogram substantially the same as the one set forth in FIG. 10;
(d) a DSC thermogram with an endotherm having an onset at about 89° C. and a peak at about 96° C.;
(e) a thermo-gravimetric analysis (TGA) thermogram substantially the same as the one set forth in FIG. 10;
(f) unit cell parameters approximately equal to the following at a temperature of approximately 100(2) K:

| Molecular formula | C₃₃H₃₂N₆O₃ | | | | |
|---|---|---|---|---|---|
| Molecular weight | 560.64 | | | | |
| Crystal system | Triclinic | | | | |
| Space group | P1 | a | 11.3552(5) Å | α | 79.657(3)° |
| | | b | 11.7741(4) Å | β | 70.352(4)° |
| | | c | 12.2064(4) Å | γ | 67.080(4)° |

| | |
|---|---|
| V | 1413.38(11) Å³ |
| Z | 2 |
| Density (calculated) | 1.317 Mg/m³ |
| Absorption coefficient | 0.699 mm⁻¹ |
| Wavelength | 1.54178 Å |
| F(000) | 592 |
| T | 100(2) K |

or
(g) combinations thereof.

In some embodiments, the crystalline form of the acetophenone solvate of Compound 1 (Form 5) has an X-ray powder diffraction (XRPD) pattern substantially the same as shown in FIG. 9. In some embodiments, the crystalline form of the acetophenone solvate of Compound 1 (Form 5) has an X-ray powder diffraction (XRPD) pattern with characteristic peaks at 7.6±0.1° 2-Theta, 8.8±0.1° 2-Theta, 15.2±0.1° 2-Theta, 17.6±0.1° 2-Theta, 18.9±0.1° 2-Theta, 19.5±0.1° 2-Theta, 20.4±0.1° 2-Theta, 21.0±0.1° 2-Theta, 21.3±0.1° 2-Theta, 21.8±0.1° 2-Theta, 24.3±0.1° 2-Theta, and 24.8±0.1° 2-Theta. In some embodiments, the crystalline form of the acetophenone solvate of Compound 1 (Form 5) has a DSC thermogram substantially the same as the one set forth in FIG. 10. In some embodiments, the crystalline form of the acetophenone solvate of Compound 1 (Form 5) has a DSC thermogram with an endotherm having an onset at about 89° C. and a peak at about 96° C. In some embodiments, the crystalline form of the acetophenone solvate of Compound 1 (Form 5) has a thermo-gravimetric analysis (TGA) thermogram substantially the same as the one set forth in FIG. 10. In some embodiments, the crystalline form of the acetophenone solvate of Compound 1 (Form 5) has a unit cell parameters approximately equal to the following at a temperature of approximately 100(2) K:

| Molecular formula | C₃₃H₃₂N₆O₃ | | | | |
|---|---|---|---|---|---|
| Molecular weight | 560.64 | | | | |
| Crystal system | Triclinic | | | | |
| Space group | P1 | a | 11.3552(5) Å | α | 79.657(3)° |
| | | b | 11.7741(4) Å | β | 70.352(4)° |
| | | c | 12.2064(4) Å | γ | 67.080(4)° |

| | |
|---|---|
| V | 1413.38(11) Å³ |
| Z | 2 |
| Density (calculated) | 1.317 Mg/m³ |
| Absorption coefficient | 0.699 mm⁻¹ |
| Wavelength | 1.54178 Å |
| F(000) | 592 |
| T | 100(2) K |

In some embodiments, the crystalline form of the acetophenone solvate of Compound 1 (Form 5) is characterized as having properties (a), (b), (c), (d), (e), and (f).

In another aspect, described herein is a chlorobenzene solvate of 1-((R)-3-(4-amino-3-(4-phenoxyphenyl)-1H-pyrazolo[3,4-d]pyrimidin-1-yl)piperidin-1-yl)prop-2-en-1-one. In another aspect, described herein is a crystalline form (Form 6) of a chlorobenzene solvate of 1-((R)-3-(4-amino-3-(4-phenoxyphenyl)-1H-pyrazolo[3,4-d]pyrimidin-1-yl)piperidin-1-yl)prop-2-en-1-one that has at least one of the following properties:
(a) an X-ray powder diffraction (XRPD) pattern substantially the same as shown in FIG. 11;
(b) an X-ray powder diffraction (XRPD) pattern with at least two, four, or all of the characteristic peaks at 18.4±0.1° 2-Theta, 19.4±0.1° 2-Theta, 20.2±0.1° 2-Theta, 20.9±0.1° 2-Theta, 21.2±0.1° 2-Theta, 21.9±0.1° 2-Theta, and 25.0±0.1° 2-Theta;
(c) substantially the same X-ray powder diffraction (XRPD) pattern post storage at 40° C. and 75% RH for at least a week;
(d) a DSC thermogram substantially the same as the one set forth in FIG. 12;
(e) a DSC thermogram with an endotherm having an onset at about 92° C. and a peak at about 95° C.;
(f) a thermo-gravimetric analysis (TGA) thermogram substantially the same as the one set forth in FIG. 12; or
(g) combinations thereof.

In some embodiments, the crystalline form of the chlorobenzene solvate of Compound 1 (Form 6) has an X-ray powder diffraction (XRPD) pattern substantially the same as shown in FIG. 11. In some embodiments, the crystalline form of the chlorobenzene solvate of Compound 1 (Form 6) has an X-ray powder diffraction (XRPD) pattern with characteristic peaks at 18.4±0.1° 2-Theta, 19.4±0.1° 2-Theta, 20.2±0.1° 2-Theta, 20.9±0.1° 2-Theta, 21.2±0.1° 2-Theta, 21.9±0.1° 2-Theta, and 25.0±0.1° 2-Theta. In some embodiments, the crystalline form of the chlorobenzene solvate of Compound 1 (Form 6) has a substantially the same X-ray powder diffraction (XRPD) pattern post storage at 40° C. and 75% RH for at least a week. In some embodiments, the crystalline form of the chlorobenzene solvate of Compound 1 (Form 6) has a DSC thermogram substantially the same as the one set forth in FIG. 12. In some embodiments, the crystalline form of the chlorobenzene solvate of Compound 1 (Form 6) has a DSC thermogram with an endotherm having an onset at about 92° C. and a peak at about 95° C. In some embodiments, the crystalline form of the chlorobenzene solvate of Compound 1 (Form 6) has a thermo-gravimetric analysis (TGA) thermogram substantially the same as the one set forth in FIG. 12. In some embodiments, the crystalline form of the chlorobenzene solvate of Compound 1 (Form 6) is characterized as having properties (a), (b), (c), (d), (e), and (f).

In another aspect, described herein is a hemi-acetophenone solvate of 1-((R)-3-(4-amino-3-(4-phenoxyphenyl)-1H-pyrazolo[3,4-d]pyrimidin-1-yl)piperidin-1-yl)prop-2-en-1-one. In another aspect, described herein is a crystalline form (Form 7) of an acetophenone solvate of 1-((R)-3-(4-amino-3-(4-phenoxyphenyl)-1H-pyrazolo[3,4-d]pyrimidin-1-yl)piperidin-1-yl)prop-2-en-1-one that has at least one of the following properties:
(a) an X-ray powder diffraction (XRPD) pattern substantially the same as shown in FIG. 13;
(b) an X-ray powder diffraction (XRPD) pattern with at least two, four, six, or all of the characteristic peaks at 6.5±0.1° 2-Theta, 13.0±0.1° 2-Theta, 17.6±0.1° 2-Theta, 18.4±0.1° 2-Theta, 19.9±0.1° 2-Theta, 21.0±0.1° 2-Theta, 21.5±0.1° 2-Theta, 22.1±0.1° 2-Theta, and 23.9±0.1° 2-Theta;
(c) a DSC thermogram substantially the same as the one set forth in FIG. 14;
(d) a DSC thermogram with an endotherm having an onset at about 124° C. and a peak at about 127° C.;
(e) a thermo-gravimetric analysis (TGA) thermogram substantially the same as the one set forth in FIG. 14;
or
(f) combinations thereof.

In some embodiments, the crystalline form of the acetophenone solvate of Compound 1 (Form 7) has an X-ray powder diffraction (XRPD) pattern substantially the same as shown in FIG. 13. In some embodiments, the crystalline form of the acetophenone solvate of Compound 1 (Form 7) has an X-ray powder diffraction (XRPD) pattern with characteristic peaks at 6.5±0.1° 2-Theta, 13.0±0.1° 2-Theta, 17.6±0.1° 2-Theta, 18.4±0.1° 2-Theta, 19.9±0.1 2-Theta, 21.0±0.1° 2-Theta, 21.5±0.1° 2-Theta, 22.1±0.1° 2-Theta, and 23.9±0.1° 2-Theta. In some embodiments, the crystalline form of the acetophenone solvate of Compound 1 (Form 7) has a DSC thermogram substantially the same as the one set forth in FIG. 14. In some embodiments, the crystalline form of the acetophenone solvate of Compound 1 (Form 7) has a DSC thermogram with an endotherm having an onset at about 124° C. and a peak at about 127° C. In some embodiments, the crystalline form of the acetophenone solvate of Compound 1 (Form 7) has a thermo-gravimetric analysis (TGA) thermogram substantially the same as the one set forth in FIG. 14. In some embodiments, the crystalline form of the acetophenone solvate of Compound 1 (Form 7) is characterized as having properties (a), (b), (c), (d), and (e).

In another aspect, described herein is a dimethylacetamide solvate of 1-((R)-3-(4-amino-3-(4-phenoxyphenyl)-1H-pyrazolo[3,4-d]pyrimidin-1-yl)piperidin-1-yl)prop-2-en-1-one. In another aspect, described herein is a crystalline form (Form 8) of a dimethylacetamide solvate of 1-((R)-3-(4-amino-3-(4-phenoxyphenyl)-1H-pyrazolo[3,4-d]pyrimidin-1-yl)piperidin-1-yl)prop-2-en-1-one that has at least one of the following properties:
(a) an X-ray powder diffraction (XRPD) pattern substantially the same as shown in FIG. 15;
(b) an X-ray powder diffraction (XRPD) pattern with at least two, four, or all of the characteristic peaks at 8.8±0.1° 2-Theta, 19.2±0.1° 2-Theta, 19.9±0.1° 2-Theta, 22.5±0.1° 2-Theta, 24.5±0.1° 2-Theta, and 25.3±0.1° 2-Theta;
(c) a DSC thermogram substantially the same as the one set forth in FIG. 16;
(d) a DSC thermogram with an endotherm having an onset at about 82° C. and a peak at about 85° C.;
(e) a thermo-gravimetric analysis (TGA) thermogram substantially the same as the one set forth in FIG. 16;
(f) unit cell parameters approximately equal to the following at a temperature of approximately 100(2) K:

| Molecular formula | C₂₉H₃₃N₇O₃ | | | | |
|---|---|---|---|---|---|
| Molecular weight | 527.62 | | | | |
| Crystal system | Triclinic | | | | |
| Space group | P1 | a | 9.3627(3) Å | α | 70.831(3)° |
| | | b | 10.9543(4) Å | β | 76.034(3)° |
| | | c | 14.7742(5) Å | γ | 70.721(3)° |

| | |
|---|---|
| V | 1335.88(9) Å³ |
| Z | 2 |
| Density (calculated) | 1.312 Mg/m³ |
| Absorption coefficient | 0.711 mm⁻¹ |
| Wavelength | 1.54178 Å |
| F(000) | 560 |
| T | 100(2) K |

or
(g) combinations thereof.

In some embodiments, the crystalline form of the dimethylacetamide solvate of Compound 1 (Form 8) has an X-ray powder diffraction (XRPD) pattern substantially the same as shown in FIG. 15. In some embodiments the crystalline form of the dimethylacetamide solvate of Compound 1 (Form 8) has an X-ray powder diffraction (XRPD) pattern with characteristic peaks at 8.8±0.1° 2-Theta, 19.2±0.1° 2-Theta, 19.9±0.1° 2-Theta, 22.5±0.10 2-Theta, 24.5±0.1° 2-Theta, and 25.3±0.1° 2-Theta. In some embodiments, the crystalline form of the dimethylacetamide solvate of Compound 1 (Form 8) has a DSC thermogram substantially the same as the one set forth in FIG. 16. In some embodiments, the crystalline form of the dimethylacetamide solvate of Compound 1 (Form 8) has a DSC thermogram with an endotherm having an onset at about 82° C. and a peak at about 85° C. In some embodiments, the crystalline form of the dimethylacetamide solvate of Compound 1 (Form 8) has a thermo-gravimetric analysis (TGA) thermogram substantially the same as the one set forth in FIG. 16. In some embodiments, the crystalline form of the dimethylacetamide solvate of Compound 1 (Form 8) is characterized as having properties (a), (b), (c), (d), (e), and (f).

In another aspect, described herein is a benzyl acetate solvate of 1-((R)-3-(4-amino-3-(4-phenoxyphenyl)-1H-pyrazolo[3,4-d]pyrimidin-1-yl)piperidin-1-yl)prop-2-en-1-one. In another aspect, described herein is a crystalline form (Form 9) of a benzyl acetate solvate of 1-((R)-3-(4-amino-3-(4-phenoxyphenyl)-1H-pyrazolo[3,4-d]pyrimidin-1-yl)piperidin-1-yl)prop-2-en-1-one that has at least one of the following properties:
(a) an X-ray powder diffraction (XRPD) pattern substantially the same as shown in FIG. 17;
(b) an X-ray powder diffraction (XRPD) pattern with at least two, four, or all of the characteristic peaks at 12.8±0.1° 2-Theta, 17.8±0.1° 2-Theta, 18.7±0.1° 2-Theta, 19.2±0.1° 2-Theta, 20.1±0.1° 2-Theta, 20.7±0.1° 2-Theta, 22.1±0.1° 2-Theta and 22.9±0.1° 2-Theta;
(c) a DSC thermogram substantially the same as the one set forth in FIG. 18;
(d) a DSC thermogram with an endotherm having an onset at about 106° C. and a peak at about 108° C. and an endotherm having an onset at about 155° C. and a peak at about 158° C.;
(e) a thermo-gravimetric analysis (TGA) thermogram substantially the same as the one set forth in FIG. 18;
or
(f) combinations thereof.

In some embodiments, the crystalline form of the benzyl acetate solvate of Compound 1 (Form 9) has an X-ray powder diffraction (XRPD) pattern substantially the same as shown in FIG. 17. In some embodiments the crystalline form of the benzyl acetate solvate of Compound 1 (Form 9) has an X-ray powder diffraction (XRPD) pattern with characteristic peaks at 12.8±0.1° 2-Theta, 17.8±0.1° 2-Theta, 18.7±0.1° 2-Theta, 19.2±0.1° 2-Theta, 20.1±0.1° 2-Theta, 20.7±0.1° 2-Theta, 21.8±0.1° 2-Theta, 22.1±0.1° 2-Theta and 22.9±0.1° 2-Theta. In some embodiments, the crystalline form of the benzyl acetate solvate of Compound 1 (Form 9) has a DSC thermogram substantially the same as the one set forth in FIG. 18. In some embodiments, the crystalline form of the benzyl acetate solvate of Compound 1 (Form 9) has a DSC thermogram with an endotherm having an onset at about 106° C. and a peak at about 108° C. and an endotherm having an onset at about 155° C. and a peak at about 158° C. In some embodiments, the crystalline form of the benzyl acetate solvate of Compound 1 (Form 9) has a thermo-gravimetric analysis (TGA) thermogram substantially the same as the one set forth in FIG. 18. In some embodiments, the crystalline form of the benzyl acetate solvate of Compound 1 (Form 9) is characterized as having properties (a), (b), (c), (d), and (e).

In another aspect, described herein is a 1,1,2-trichloroethane solvate of 1-((R)-3-(4-amino-3-(4-phenoxyphenyl)-1H-pyrazolo[3,4-d]pyrimidin-1-yl)piperidin-1-yl)prop-2-en-1-one. In another aspect, described herein is a crystalline form (Form 10) of a 1,1,2-trichloroethane solvate of 1-((R)-3-(4-amino-3-(4-phenoxyphenyl)-1H-pyrazolo[3,4-d]pyrimidin-1-yl)piperidin-1-yl)prop-2-en-1-one that has at least one of the following properties:
(a) an X-ray powder diffraction (XRPD) pattern substantially the same as shown in FIG. 19;
(b) an X-ray powder diffraction (XRPD) pattern with at least two, four, or all of the characteristic peaks at 5.4±0.1° 2-Theta, 18.6±0.1° 2-Theta, 20.1±0.1° 2-Theta, 20.8±0.1° 2-Theta, 21.3±0.1° 2-Theta, 21.7±0.1° 2-Theta, and 22.6±0.1° 2-Theta;
(c) a DSC thermogram substantially the same as the one set forth in FIG. 20;
(d) a DSC thermogram with an endotherm having an onset at about 150° C. and a peak at about 154° C.;
(e) a thermo-gravimetric analysis (TGA) thermogram substantially the same as the one set forth in FIG. 20;
or
(f) combinations thereof.

In some embodiments, the crystalline form of the 1,1,2-trichloroethane solvate of Compound 1 (Form 10) has an X-ray powder diffraction (XRPD) pattern substantially the same as shown in FIG. 19. In some embodiments the crystalline form of the 1,1,2-trichloroethane solvate of Compound 1 (Form 10) has an X-ray powder diffraction (XRPD) pattern with characteristic peaks at 5.4±0.1° 2-Theta, 18.6±0.1° 2-Theta, 20.1±0.1° 2-Theta, 20.8±0.1° 2-Theta, 21.3±0.1° 2-Theta, 21.7±0.1° 2-Theta, and 22.6±0.1° 2-Theta. In some embodiments, the crystalline form of the 1,1,2-trichloroethane solvate of Compound 1 (Form 10) has a DSC thermogram substantially the same as the one set forth in FIG. 20. In some embodiments, the crystalline form of the 1,1,2-trichloroethane solvate of Compound 1 (Form 10) has a DSC thermogram with an endotherm having an onset at about 150° C. and a peak at about 154° C. In some embodiments, the crystalline form of the 1,1,2-trichloroethane solvate of Compound 1 (Form 10) has a thermo-gravimetric analysis (TGA) thermogram substantially the same as the one set forth in FIG. 20. In some embodiments, the crystalline form of the 1,1,2-trichloroethane solvate of Compound 1 (Form 10) is characterized as having properties (a), (b), (c), (d), and (e).

In another aspect, described herein is a pharmaceutically acceptable salt of 1-((R)-3-(4-amino-3-(4-phenoxyphenyl)-1H-pyrazolo[3,4-d]pyrimidin-1-yl)piperidin-1-yl)prop-2-en-1-one, wherein the pharmaceutically acceptable salt is an acid addition salt. In some embodiments, the pharmaceutically acceptable salt is amorphous. In some embodiments, the pharmaceutically acceptable salt is crystalline.

In a further aspect are provided pharmaceutical compositions, which include a solvate of 1-((R)-3-(4-amino-3-(4-phenoxyphenyl)-1H-pyrazolo[3,4-d]pyrimidin-1-yl)piperidin-1-yl)prop-2-en-1-one as described herein, and at least one additional ingredient selected from pharmaceutically acceptable carriers, diluents and excipients. In some embodiments, the pharmaceutical composition comprises a crystalline form of a butyronitrile solvate of 1-((R)-3-(4-amino-3-(4-phenoxyphenyl)-1H-pyrazolo[3,4-d]pyrimidin-1-yl)piperidin-1-yl)prop-2-en-1-one. In some embodiments, the pharmaceutical composition comprises a crystalline form of a 1,2-dimethoxyethane solvate of 1-((R)-3-(4-amino-3-(4-phenoxyphenyl)-1H-pyrazolo[3,4-d]pyrimidin-1-yl)piperidin-1-yl)prop-2-en-1-one. In some embodiments, the pharmaceutical composition comprises a crystalline form of a hexafluorobenzene solvate of 1-((R)-3-(4-amino-3-(4-phenoxyphenyl)-1H-pyrazolo[3,4-d]pyrimidin-1-yl)piperidin-1-yl)prop-2-en-1-one. In some embodiments, the pharmaceutical composition comprises a crystalline form of a chlorobenzene solvate of 1-((R)-3-(4-amino-3-(4-phenoxyphenyl)-1H-pyrazolo[3,4-d]pyrimidin-1-yl)piperidin-1-yl)prop-2-en-1-one. In some embodiments, the pharmaceutical composition comprises a crystalline form of an acetophenone solvate of 1-((R)-3-(4-amino-3-(4-phenoxyphenyl)-1H-pyrazolo[3,4-d]pyrimidin-1-yl)piperidin-1-yl)prop-2-en-1-one. In some embodiments, the pharmaceutical composition comprises a crystalline form of a dimethylacetamide solvate of 1-((R)-3-(4-amino-3-(4-phenoxyphenyl)-1H-pyrazolo[3,4-d]pyrimidin-1-yl)piperidin-1-yl)prop-2-en-1-one. In some embodiments, the pharmaceutical composition comprises a crystalline form of a benzyl acetate solvate of 1-((R)-3-(4-amino-3-(4-phenoxyphenyl)-1H-pyrazolo[3,4-d]pyrimidin-1-yl)piperidin-1-yl)prop-2-en-1-one. In some embodiments, the pharmaceutical composition comprises a crystalline form of a 1,1,2-trichloroethane solvate of 1-((R)-3-(4-amino-3-(4-phenoxyphenyl)-1H-pyrazolo[3,4-d]pyrimidin-1-yl)piperidin-1-yl)prop-2-en-1-one. In some embodiments, the pharmaceutical composition is in a form suitable for oral administration to a mammal. In some embodiments, the pharmaceutical composition is an oral solid dosage form. In some embodiments, the pharmaceutical composition comprises about 0.5 mg to about 1000 mg of 1-((R)-3-(4-amino-3-(4-phenoxyphenyl)-1H-pyrazolo[3,4-d]pyrimidin-1-yl)piperidin-1-yl)prop-2-en-1-one solvate.

In another aspect, provided herein are methods for treating a patient by administering a solvate of 1-((R)-3-(4-amino-3-(4-phenoxyphenyl)-1H-pyrazolo[3,4-d]pyrimidin-1-yl)piperidin-1-yl)prop-2-en-1-one. In some embodiments, provided herein is a method of inhibiting the activity of tyrosine kinase(s), such as Btk, or of treating a disease, disorder, or condition, which would benefit from inhibition of tyrosine kinase(s), such as Btk, in a mammal, which includes administering to the mammal a therapeutically effective amount of a solvate of 1-((R)-3-(4-amino-3-(4-phenoxyphenyl)-1H-pyrazolo[3,4-d]pyrimidin-1-yl)piperidin-1-yl)prop-2-en-1-one.

In another aspect, provided herein is the use of a solvate of 1-((R)-3-(4-amino-3-(4-phenoxyphenyl)-1H-pyrazolo[3,4-d]pyrimidin-1-yl)piperidin-1-yl)prop-2-en-1-one for inhibiting Bruton's tyrosine kinase (Btk) activity or for the treatment of a disease, disorder, or condition, which would benefit from inhibition of Bruton's tyrosine kinase (Btk) activity.

In some embodiments, a solvate of 1-((R)-3-(4-amino-3-(4-phenoxyphenyl)-1H-pyrazolo[3,4-d]pyrimidin-1-yl is orally administered.

In other embodiments, a solvate of 1-((R)-3-(4-amino-3-(4-phenoxyphenyl)-1H-pyrazolo[3,4-d]pyrimidin-1-yl)piperidin-1-yl)prop-2-en-1-one is used for the formulation of a medicament for the inhibition of tyrosine kinase activity. In some other embodiments, a solvate of 1-((R)-3-(4-amino-3-(4-phenoxyphenyl)-1H-pyrazolo[3,4-d]pyrimidin-1-yl)piperidin-1-yl)prop-2-en-1-one is used for the formulation of a medicament for the inhibition of Bruton's tyrosine kinase (Btk) activity.

In another aspect, provided herein is a method of treating cancer in a mammal comprising administering to the mammal a pharmaceutical composition described herein comprising a solvate of 1-((R)-3-(4-amino-3-(4-phenoxyphenyl)-1H-pyrazolo[3,4-d]pyrimidin-1-yl)piperidin-1-yl)prop-2-en-1-one. In some embodiments, the cancer is a B cell malignancy. In some embodiments, the cancer is a B cell malignancy selected from chronic lymphocytic leukemia (CLL)/small lymphocytic lymphoma (SLL), mantle cell lymphoma (MCL), diffuse large B Cell lymphoma (DLBCL), and multiple myeloma. In some embodiments, the cancer is a lymphoma, leukemia or a solid tumor. In some embodiments, the cancer is diffuse large B cell lymphoma, follicular lymphoma, chronic lymphocytic lymphoma, chronic lymphocytic leukemia, B-cell prolymphocytic leukemia, lymphoplasmacytic lymphoma/Waldenström macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, where the subject is suffering from a cancer, an anti-cancer agent is administered to the subject in addition to one of the above-mentioned compounds. In one embodiment, the anti-cancer agent is an inhibitor of mitogen-activated protein kinase signaling.

In another aspect, provided herein is a method of treating an inflammatory or an autoimmune disease in a mammal comprising administering to the mammal a pharmaceutical composition described herein comprising a solvate of 1-((R)-3-(4-amino-3-(4-phenoxyphenyl)-1H-pyrazolo[3,4-d]pyrimidin-1-yl)piperidin-1-yl)prop-2-en-1-one. In some embodiments, the inflammatory disease is asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis. In some embodiments, the autoimmune disease is inflammatory bowel disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease Sjtgren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, autoimmune hemolytic anemia, warm autoimmune hemolytic anemia, cold hemolytic anemia, Wegener's granulomatosis, psoriasis, alopecia universalis, Behqet's disease, chronic fatigue, dysautonomia, endometriosis, interstitial cystitis, neuromyotonia, scleroderma, or vulvodynia.

In some embodiments, the composition and methods described herein can be used to treat ischemia/reperfusion injury, such as ischemia/reperfusion injury caused by transplantation, heart attack, stroke, or the like.

Articles of manufacture including packaging material, a solvate of 1-((R)-3-(4-amino-3-(4-phenoxyphenyl)-1H-pyrazolo[3,4-d]pyrimidin-1-yl)piperidin-1-yl)prop-2-en-1-one within the packaging material, and a label that indicates that a solvate of 1-((R)-3-(4-amino-3-(4-phenoxyphenyl)-1H-pyrazolo[3,4-d]pyrimidin-1-yl)piperidin-1-yl)prop-2-en-1-one is used for inhibiting the activity of tyrosine kinase(s), such as Btk, are provided.

In a further aspect, provided herein is a method of treating an autoimmune disease in a mammal, comprising administering a solvate of 1-((R)-3-(4-amino-3-(4-phenoxyphenyl)-1H-pyrazolo[3,4-d]pyrimidin-1-yl)piperidin-1-yl)prop-2-en-1-one to the mammal.

In a further aspect, provided herein is a method of treating a heteroimmune disease or condition in a mammal, comprising administering a solvate of 1-((R)-3-(4-amino-3-(4-phenoxyphenyl)-1H-pyrazolo[3,4-d]pyrimidin-1-yl)piperidin-1-yl)prop-2-en-1-one to the mammal.

In a further aspect, provided herein is a method of treating an inflammatory disease in a mammal, comprising administering a solvate of 1-((R)-3-(4-amino-3-(4-phenoxyphenyl)-1H-pyrazolo[3,4-d]pyrimidin-1-yl)piperidin-1-yl)prop-2-en-1-one to the mammal.

In a further aspect, provided herein is a method of treating cancer in a mammal, comprising administering a solvate of 1-((R)-3-(4-amino-3-(4-phenoxyphenyl)-1H-pyrazolo[3,4-d]pyrimidin-1-yl)piperidin-1-yl)prop-2-en-1-one to the mammal.

In a further aspect, provided herein is a method of treating a thromboembolic disorder in a mammal, comprising administering a solvate of 1-((R)-3-(4-amino-3-(4-phenoxyphenyl)-1H-pyrazolo[3,4-d]pyrimidin-1-yl)piperidin-1-yl)prop-2-en-1-one to the mammal. Thromboembolic disorders include, but are not limited to, myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In another aspect are methods for modulating, including irreversibly inhibiting the activity of Btk or other tyrosine kinases, wherein the other tyrosine kinases share homology with Btk by having a cysteine residue (including a Cys 481 residue) that can form a covalent bond with a solvate of 1-((R)-3-(4-amino-3-(4-phenoxyphenyl)-1H-pyrazolo[3,4-d]pyrimidin-1-yl)piperidin-1-yl)prop-2-en-1-one, in a mammal comprising administering to the mammal at least once an effective amount of a solvate of 1-((R)-3-(4-amino-3-(4-phenoxyphenyl)-1H-pyrazolo[3,4-d]pyrimidin-1-yl)piperidin-1-yl)prop-2-en-1-one. In another aspect are methods for modulating, including irreversibly inhibiting, the activity of Btk in a mammal comprising administering to the mammal at least once an effective amount of a solvate of 1-((R)-3-(4-amino-3-(4-phenoxyphenyl)-1H-pyrazolo[3,4-d]pyrimidin-1-yl)piperidin-1-yl)prop-2-en-1-one. In another aspect are methods for treating Btk-dependent or Btk mediated conditions or diseases, comprising administering to the mammal at least once an effective amount of a solvate of 1-((R)-3-(4-amino-3-(4-phenoxyphenyl)-1H-pyrazolo[3,4-d]pyrimidin-1-yl)piperidin-1-yl)prop-2-en-1-one.

In another aspect are methods for treating inflammation comprising administering to the mammal at least once an effective amount of a solvate of 1-((R)-3-(4-amino-3-(4-phenoxyphenyl)-1H-pyrazolo[3,4-d]pyrimidin-1-yl)piperidin-1-yl)prop-2-en-1-one.

A further aspect are methods for the treatment of cancer comprising administering to the mammal at least once an effective amount of a solvate of 1-((R)-3-(4-amino-3-(4-phenoxyphenyl)-1H-pyrazolo[3,4-d]pyrimidin-1-yl)piperidin-1-yl)prop-2-en-1-one. The type of cancer may include, but is not limited to, pancreatic cancer and other solid or hematological tumors.

In another aspect are methods for treating respiratory diseases comprising administering to the mammal at least once an effective amount of a solvate of 1-((R)-3-(4-amino-3-(4-phenoxyphenyl)-1H-pyrazolo[3,4-d]pyrimidin-1-yl)piperidin-1-yl)prop-2-en-1-one. In a further embodiment of this aspect, the respiratory disease is asthma. In a further embodiment of this aspect, the respiratory disease includes, but is not limited to, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma.

In another aspect are methods for preventing rheumatoid arthritis and/or osteoarthritis comprising administering to the mammal at least once an effective amount of a solvate of 1-((R)-3-(4-amino-3-(4-phenoxyphenyl)-1H-pyrazolo[3,4-d]pyrimidin-1-yl)piperidin-1-yl)prop-2-en-1-one.

In another aspect are methods for treating inflammatory responses of the skin comprising administering to the mammal at least once an effective amount of a solvate of 1-((R)-3-(4-amino-3-(4-phenoxyphenyl)-1H-pyrazolo[3,4-d]pyrimidin-1-yl)piperidin-1-yl)prop-2-en-1-one. Such inflammatory responses of the skin include, by way of example, dermatitis, contact dermatitis, eczema, urticaria, rosacea, and scarring. In another aspect are methods for reducing psoriatic lesions in the skin, joints, or other tissues or organs, comprising administering to the mammal an effective amount of a solvate of 1-((R)-3-(4-amino-3-(4-phenoxyphenyl)-1H-pyrazolo[3,4-d]pyrimidin-1-yl)piperidin-1-yl)prop-2-en-1-one.

In another aspect is the use of a solvate of 1-((R)-3-(4-amino-3-(4-phenoxyphenyl)-1H-pyrazolo[3,4-d]pyrimidin-1-yl)piperidin-1-yl)prop-2-en-1-one in the manufacture of a medicament for treating an inflammatory disease or condition in an animal in which the activity of Btk or other tyrosine kinases, wherein the other tyrosine kinases share homology with Btk by having a cysteine residue (including a Cys 481 residue) that can form a covalent bond with at least one irreversible inhibitor described herein, contributes to the pathology and/or symptoms of the disease or condition. In one embodiment of this aspect, the tyrosine kinase protein is Btk. In another or further embodiment of this aspect, the inflammatory disease or conditions are respiratory, cardiovascular, or proliferative diseases.

In any of the aforementioned aspects are further embodiments in which a solvate of 1-((R)-3-(4-amino-3-(4-phenoxyphenyl)-1H-pyrazolo[3,4-d]pyrimidin-1-yl)piperidin-1-yl)prop-2-en-1-one is (a) systemically administered to the mammal; (b) administered orally to the mammal; (c) intravenously administered to the mammal; (d) administered by inhalation; (e) administered by nasal administration; or (f) administered by injection to the mammal; (g) administered topically (dermal) to the mammal; (h) administered by ophthalmic administration; or (i) administered rectally to the mammal.

In any of the aforementioned aspects are further embodiments comprising single administration of a solvate of 1-((R)-3-(4-amino-3-(4-phenoxyphenyl)-1H-pyrazolo[3,4-d]pyrimidin-1-yl)piperidin-1-yl)prop-2-en-1-one, including further embodiments in which a solvate of 1-((R)-3-(4-amino-3-(4-phenoxyphenyl)-1H-pyrazolo[3,4-d]pyrimidin-1-yl)piperidin-1-yl)prop-2-en-1-one is administered (i) once; (ii) multiple times over the span of one day; (iii) continually; or (iv) continuously.

In any of the aforementioned aspects are further embodiments comprising multiple administrations of a solvate of 1-((R)-3-(4-amino-3-(4-phenoxyphenyl)-1H-pyrazolo[3,4-d]pyrimidin-1-yl)piperidin-1-yl)prop-2-en-1-one, including further embodiments in which (i) a solvate of 1-((R)-3-(4-amino-3-(4-phenoxyphenyl)-1H-pyrazolo[3,4-d]pyrimidin-1-yl)piperidin-1-yl)prop-2-en-1-one is administered in a single dose; (ii) the time between multiple administrations is every 6 hours; (iii) a solvate of 1-((R)-3-(4-amino-3-(4-phenoxyphenyl)-1H-pyrazolo[3,4-d]pyrimidin-1-yl)piperidin-1-yl)prop-2-en-1-one is administered to the mammal every 8 hours. In further or alternative embodiments, the method comprises a drug holiday, wherein the administration of a solvate of 1-((R)-3-(4-amino-3-(4-phenoxyphenyl)-1H-pyrazolo[3,4-d]pyrimidin-1-yl)piperidin-1-yl)prop-2-en-1-one is temporarily suspended or the dose of a solvate of 1-((R)-3-(4-amino-3-(4-phenoxyphenyl)-1H-pyrazolo[3,4-d]pyrimidin-1-yl)piperidin-1-yl)prop-2-en-1-one being administered is temporarily reduced; at the end of the drug holiday, dosing of a solvate of 1-((R)-3-(4-amino-3-(4-phenoxyphenyl)-1H-pyrazolo[3,4-d]pyrimidin-1-yl)piperidin-1-yl)prop-2-en-1-one is resumed. The length of the drug holiday can vary from 2 days to 1 year.

In some embodiments, in any of the embodiments disclosed herein (including methods, uses, formulations, combination therapy, etc.), a solvate of 1-((R)-3-(4-amino-3-(4-phenoxyphenyl)-1H-pyrazolo[3,4-d]pyrimidin-1-yl)piperidin-1-yl)prop-2-en-1-one is: optically pure (i.e. greater than 99% chiral purity by HPLC). In some embodiments, in any of the embodiments disclosed herein (including methods, uses, formulations, combination therapy, etc.), the solvate of 1-((R)-3-(4-amino-3-(4-phenoxyphenyl)-1H-pyrazolo[3,4-d]pyrimidin-1-yl)piperidin-1-yl)prop-2-en-1-one is replaced with: a) a solvate of 1-((R)-3-(4-amino-3-(4-phenoxyphenyl)-1H-pyrazolo[3,4-d]pyrimidin-1-yl)piperidin-1-yl)prop-2-en-1-one of lower chiral purity; b) a solvate of 1-((S)-3-(4-amino-3-(4-phenoxyphenyl)-1H-pyrazolo[3,4-d]pyrimidin-1-yl)piperidin-1-yl)prop-2-en-1-one of any optical purity; or c) a racemic solvate of 1-((R)-3-(4-amino-3-(4-phenoxyphenyl)-1H-pyrazolo[3,4-d]pyrimidin-1-yl)piperidin-1-yl)prop-2-en-1-one.

In any of the embodiments disclosed herein (including methods, uses, formulations, combination therapy, etc.), an amorphous form of a solvate of 1-((R)-3-(4-amino-3-(4-phenoxyphenyl)-1H-pyrazolo[3,4-d]pyrimidin-1-yl)piperidin-1-yl)prop-2-en-1-one is used. In any of the embodiments disclosed herein (including methods, uses, formulations, combination therapy, etc.), a crystalline form of a solvate of 1-((R)-3-(4-amino-3-(4-phenoxyphenyl)-1H-pyrazolo[3,4-d]pyrimidin-1-yl)piperidin-1-yl)prop-2-en-1-one is used.

In any of the embodiments disclosed herein (including methods, uses, formulations, combination therapy, etc.), a crystalline form of a butyronitrile solvate of 1-((R)-3-(4-amino-3-(4-phenoxyphenyl)-1H-pyrazolo[3,4-d]pyrimidin-1-yl)piperidin-1-yl)prop-2-en-1-one is used. In any of the embodiments disclosed herein (including methods, uses, formulations, combination therapy, etc.), a crystalline form of a 1,2-dimethoxyethane solvate of 1-((R)-3-(4-amino-3-(4-phenoxyphenyl)-1H-pyrazolo[3,4-d]pyrimidin-1-yl)piperidin-1-yl)prop-2-en-1-one is used. In any of the embodiments disclosed herein (including methods, uses, formulations, combination therapy, etc.), a crystalline form of a hexafluorobenzene solvate of 1-((R)-3-(4-amino-3-(4-phenoxyphenyl)-1H-pyrazolo[3,4-d]pyrimidin-1-yl)piperidin-1-yl)prop-2-en-1-one is used. In any of the embodiments disclosed herein (including methods, uses, formulations, combination therapy, etc.), a crystalline form of an acetophenone solvate of 1-((R)-3-(4-amino-3-(4-phenoxyphenyl)-1H-pyrazolo[3,4-d]pyrimidin-1-yl)piperidin-1-yl)prop-2-en-1-one is used. In any of the embodiments disclosed herein (including methods, uses, formulations, combination therapy, etc.), a crystalline form of a chlorobenzene solvate of 1-((R)-3-(4-amino-3-(4-phenoxyphenyl)-1H-pyrazolo[3,4-d]pyrimidin-1-yl)piperidin-1-yl)prop-2-en-1-one is used. In any of the embodiments disclosed herein (including methods, uses, formulations, combination therapy, etc.), a crystalline form of a dimethylacetamide solvate of 1-((R)-3-(4-amino-3-(4-phenoxyphenyl)-1H-pyrazolo[3,4-d]pyrimidin-1-yl)piperidin-1-yl)prop-2-en-1-one is used. In any of the embodiments disclosed herein (including methods, uses, formulations, combination therapy, etc.), a crystalline form of a benzyl acetate solvate of 1-((R)-3-(4-amino-3-(4-phenoxyphenyl)-1H-pyrazolo[3,4-d]pyrimidin-1-yl)piperidin-1-yl)prop-2-en-1-one is used. In any of the embodiments disclosed herein (including methods, uses, formulations, combination therapy, etc.), a crystalline form of a 1,1,2-trichloroethane solvate of 1-((R)-3-(4-amino-3-(4-phenoxyphenyl)-1H-pyrazolo[3,4-d]pyrimidin-1-yl)piperidin-1-yl)prop-2-en-1-one is used.

In some embodiments, in any of the embodiments disclosed herein (including methods, uses, formulations, combination therapy, etc.), a solvate of 1-((R)-3-(4-amino-3-(4-phenoxyphenyl)-1H-pyrazolo[3,4-d]pyrimidin-1-yl)piperidin-1-yl)prop-2-en-1-one, or a pharmaceutically acceptable salt thereof, is replaced with an active metabolite of 1-((R)-3-(4-amino-3-(4-phenoxyphenyl)-1H-pyrazolo[3,4-d]pyrimidin-1-yl)piperidin-1-yl)prop-2-en-1-one. In some embodiments, the active metabolite is in a crystalline form. In some embodiments, the active metabolite is in an amorphous phase. In further embodiments the metabolite is isolated. In some embodiments, in any of the embodiments disclosed herein (including methods, uses, formulations, combination therapy, etc.), a solvate of 1-((R)-3-(4-amino-3-(4-phenoxyphenyl)-1H-pyrazolo[3,4-d]pyrimidin-1-yl)piperidin-1-yl)prop-2-en-1-one, or a pharmaceutically acceptable salt thereof, is replaced with a prodrug of 1-((R)-3-(4-amino-3-(4-phenoxyphenyl)-1H-pyrazolo[3,4-d]pyrimidin-1-yl)piperidin-1-yl)prop-2-en-1-one, or a deuterated analog of 1-((R)-3-(4-amino-3-(4-phenoxyphenyl)-1H-pyrazolo[3,4-d]pyrimidin-1-yl)piperidin-1-yl)prop-2-en-1-one, or a pharmaceutically acceptable salt thereof.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### INCORPORATION BY REFERENCE

All publications and patent applications mentioned in this specification are herein incorporated by reference to the extent applicable and relevant.

### BRIEF DESCRIPTION OF THE FIGURES

FIG. 1. Illustrates an X-ray powder diffraction (XRPD) pattern of a crystalline form of a butyronitrile solvate of 1-((R)-3-(4-amino-3-(4-phenoxyphenyl)-1H-pyrazolo[3,4-d]pyrimidin-1-yl)piperidin-1-yl)prop-2-en-1-one (Form 1).

FIG. 2. Illustrates a DSC thermogram and a thermo-gravimetric analysis (TGA) thermogram of a crystalline form of a butyronitrile solvate of 1-((R)-3-(4-amino-3-(4-phenoxyphenyl)-1H-pyrazolo[3,4-d]pyrimidin-1-yl)piperidin-1-yl)prop-2-en-1-one (Form 1).

FIG. 3. Illustrates an X-ray powder diffraction (XRPD) pattern of a crystalline form of a 1,2-dimethoxyethane solvate of 1-((R)-3-(4-amino-3-(4-phenoxyphenyl)-1H-pyrazolo[3,4-d]pyrimidin-1-yl)piperidin-1-yl)prop-2-en-1-one (Form 2).

FIG. 4. Illustrates a DSC thermogram and a thermo-gravimetric analysis (TGA) thermogram of a crystalline form of a 1,2-dimethoxyethane solvate of 1-((R)-3-(4-amino-3-(4-phenoxyphenyl)-1H-pyrazolo[3,4-d]pyrimidin-1-yl)piperidin-1-yl)prop-2-en-1-one (Form 2).

FIG. 5. Illustrates an X-ray powder diffraction (XRPD) pattern of a crystalline form of a hexafluorobenzene solvate of 1-((R)-3-(4-amino-3-(4-phenoxyphenyl)-1H-pyrazolo[3,4-d]pyrimidin-1-yl)piperidin-1-yl)prop-2-en-1-one (Form 3).

FIG. 6. Illustrates a DSC thermogram of a crystalline form of a hexafluorobenzene solvate of 1-((R)-3-(4-amino-3-(4-phenoxyphenyl)-1H-pyrazolo[3,4-d]pyrimidin-1-yl)piperidin-1-yl)prop-2-en-1-one (Form 3).

FIG. 7. Illustrates an X-ray powder diffraction (XRPD) pattern of a crystalline form of a hexafluorobenzene solvate of 1-((R)-3-(4-amino-3-(4-phenoxyphenyl)-1H-pyrazolo[3,4-d]pyrimidin-1-yl)piperidin-1-yl)prop-2-en-1-one (Form 4).

FIG. 8. Illustrates a DSC thermogram and a thermo-gravimetric analysis (TGA) thermogram a crystalline form of a hexafluorobenzene solvate of 1-((R)-3-(4-amino-3-(4-phenoxyphenyl)-1H-pyrazolo[3,4-d]pyrimidin-1-yl)piperidin-1-yl)prop-2-en-1-one (Form 4).

FIG. 9. Illustrates an X-ray powder diffraction (XRPD) pattern of a crystalline form of an acetophenone solvate of 1-((R)-3-(4-amino-3-(4-phenoxyphenyl)-1H-pyrazolo[3,4-d]pyrimidin-1-yl)piperidin-1-yl)prop-2-en-1-one (Form 5).

FIG. 10. Illustrates a DSC thermogram and a thermo-gravimetric analysis (TGA) thermogram of a crystalline form of an acetophenone solvate of 1-((R)-3-(4-amino-3-(4-phenoxyphenyl)-1H-pyrazolo[3,4-d]pyrimidin-1-yl)piperidin-1-yl)prop-2-en-1-one (Form 5).

FIG. 11. Illustrates an X-ray powder diffraction (XRPD) pattern of a crystalline form of a chlorobenzene solvate of 1-((R)-3-(4-amino-3-(4-phenoxyphenyl)-1H-pyrazolo[3,4-d]pyrimidin-1-yl)piperidin-1-yl)prop-2-en-1-one (Form 6).

FIG. 12. Illustrates a DSC thermogram and a thermo-gravimetric analysis (TGA) thermogram of a crystalline form of a chlorobenzene solvate of 1-((R)-3-(4-amino-3-(4-phenoxyphenyl)-1H-pyrazolo[3,4-d]pyrimidin-1-yl)piperidin-1-yl)prop-2-en-1-one (Form 6).

FIG. 13. Illustrates an X-ray powder diffraction (XRPD) pattern of a crystalline form of an acetophenone solvate of 1-((R)-3-(4-amino-3-(4-phenoxyphenyl)-1H-pyrazolo[3,4-d]pyrimidin-1-yl)piperidin-1-yl)prop-2-en-1-one (Form 7).

FIG. 14. Illustrates a DSC thermogram and a thermo-gravimetric analysis (TGA) thermogram of a crystalline form of an acetophenone solvate of 1-((R)-3-(4-amino-3-(4-phenoxyphenyl)-1H-pyrazolo[3,4-d]pyrimidin-1-yl)piperidin-1-yl)prop-2-en-1-one (Form 7).

FIG. 15. Illustrates an X-ray powder diffraction (XRPD) pattern of a crystalline form of a dimethylacetamide solvate of 1-((R)-3-(4-amino-3-(4-phenoxyphenyl)-1H-pyrazolo[3,4-d]pyrimidin-1-yl)piperidin-1-yl)prop-2-en-1-one (Form 8).

FIG. 16. Illustrates a DSC thermogram and a thermo-gravimetric analysis (TGA) thermogram of a crystalline form of a dimethylacetamide solvate of 1-((R)-3-(4-amino-3-(4-phenoxyphenyl)-1H-pyrazolo[3,4-d]pyrimidin-1-yl)piperidin-1-yl)prop-2-en-1-one (Form 8).

FIG. 17. Illustrates an X-ray powder diffraction (XRPD) pattern of a crystalline form of a benzyl acetate solvate of 1-((R)-3-(4-amino-3-(4-phenoxyphenyl)-1H-pyrazolo[3,4-d]pyrimidin-1-yl)piperidin-1-yl)prop-2-en-1-one (Form 9).

FIG. 18. Illustrates a DSC thermogram and a thermo-gravimetric analysis (TGA) thermogram of crystalline of a benzyl acetate solvate of 1-((R)-3-(4-amino-3-(4-phenoxyphenyl)-1H-pyrazolo[3,4-d]pyrimidin-1-yl)piperidin-1-yl)prop-2-en-1-one (Form 9).

FIG. 19. Illustrates an X-ray powder diffraction (XRPD) pattern of a crystalline form of a 1,1,2-trichloroethane solvate of 1-((R)-3-(4-amino-3-(4-phenoxyphenyl)-1H-pyrazolo[3,4-d]pyrimidin-1-yl)piperidin-1-yl)prop-2-en-1-one (Form 10).

FIG. 20. Illustrates a DSC thermogram and a thermo-gravimetric analysis (TGA) thermogram of a crystalline form of a 1,1,2-trichloroethane solvate of 1-((R)-3-(4-amino-3-(4-phenoxyphenyl)-1H-pyrazolo[3,4-d]pyrimidin-1-yl)piperidin-1-yl)prop-2-en-1-one (Form 10).

### DETAILED DESCRIPTION OF THE INVENTION

The diverse roles played by Btk signaling in various hematopoietic cell functions, e.g., B-cell receptor activation, suggests that small molecule Btk inhibitors, such as Compound 1, are useful for reducing the risk of or treating a variety of diseases affected by or affecting many cell types of the hematopoetic lineage including, e.g., autoimmune diseases, heteroimmune conditions or diseases, inflammatory diseases, cancer (e.g., B-cell proliferative disorders), and thromboembolic disorders. Further, irreversible Btk inhibitor compounds, such as Compound 1, can be used to inhibit a small subset of other tyrosine kinases that share homology with Btk by having a cysteine residue (including a Cys 481 residue) that can form a covalent bond with the irreversible inhibitor.

In some embodiments, a solvate of 1-((R)-3-(4-amino-3-(4-phenoxyphenyl)-1H-pyrazolo[3,4-d]pyrimidin-1-yl)piperidin-1-yl)prop-2-en-1-one (Compound 1) can be used in the treatment of an autoimmune disease in a mammal, which includes, but is not limited to, rheumatoid arthritis, psoriatic arthritis, osteoarthritis, Still's disease, juvenile arthritis, lupus, diabetes, myasthenia gravis, Hashimoto's thyroiditis, Ord's thyroiditis, Graves' disease Sjögren's syndrome, multiple sclerosis, Guillain-Barre syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behqet's disease, chronic fatigue, dysautonomia, endometriosis, interstitial cystitis, neuromyotonia, scleroderma, and vulvodynia.

In some embodiments, a solvate of Compound 1 can be used in the treatment of a heteroimmune disease or condition in a mammal, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

In some embodiments, a solvate of Compound 1 can be used in the treatment of an inflammatory disease in a mammal, which includes, but is not limited to asthma, inflammatory bowel disease, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, and vulvitis.

In some embodiments, a solvate of Compound 1 described herein can be used to treat hematological malignancies such as, but not limited to, a leukemia, a lymphoma, a myeloma, a non-Hodgkin's lymphoma, a Hodgkin's lymphoma, a T-cell malignancy, or a B-cell malignancy. In some embodiments, the hematological malignancy is a treatment naïve hematological malignancy. In some embodiments the hematological malignancy is a relapsed or refractory hematological malignancy.

In yet other embodiments, the methods described herein can be used to treat a cancer, e.g., B-cell proliferative disorders, which include, but are not limited to diffuse large B cell lymphoma, follicular lymphoma, chronic lymphocytic lymphoma, chronic lymphocytic leukemia, B-cell prolymphocytic leukemia, lymphoplasmacytic lymphoma/Waldenström macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, primary mediastinal B-cell lymphoma (PMBL), lymphoplasmacytic lymphoma, B cell prolymphocytic leukemia, intravascular large B cell lymphoma, primary effusion lymphoma, burkitt lymphoma/leukemia, and lymphomatoid granulomatosis.

In further embodiments, the methods described herein can be used to treat thromboembolic disorders, which include, but are not limited to myocardial infarct, angina pectoris (including unstable angina), reocclusions or restenoses after angioplasty or aortocoronary bypass, stroke, transitory ischemia, peripheral arterial occlusive disorders, pulmonary embolisms, and deep venous thromboses.

### Hematological Malignancies

Disclosed herein, in certain embodiments, is a method for treating a hematological malignancy in an individual in need thereof, comprising: administering to the individual an amount of a solvate of Compound 1.

In some embodiments, the hematological malignancy is a non-Hodgkin's lymphoma (NHL). In some embodiments, the hematological malignancy is a chronic lymphocytic leukemia (CLL), small lymphocytic lymphoma (SLL), high risk CLL, or a non-CLL/SLL lymphoma. In some embodiments, the hematological malignancy is follicular lymphoma (FL), diffuse large B-cell lymphoma (DLBCL), mantle cell lymphoma (MCL), Waldenstrom's macroglobulinemia, multiple myeloma (MM), marginal zone lymphoma, Burkitt's lymphoma, non-Burkitt high grade B cell lymphoma, or extranodal marginal zone B cell lymphoma. In some embodiments, the hematological malignancy is acute or chronic myelogenous (or myeloid) leukemia, myelodysplastic syndrome, acute lymphoblastic leukemia, or precursor B-cell acute lymphoblastic leukemia. In some embodiments, the hematological malignancy is chronic lymphocytic leukemia (CLL). In some embodiments, the hematological malignancy is mantle cell lymphoma (MCL). In some embodiments, the hematological malignancy is diffuse large B-cell lymphoma (DLBCL). In some embodiments, the hematological malignancy is diffuse large B-cell lymphoma (DLBCL), ABC subtype. In some embodiments, the hematological malignancy is diffuse large B-cell lymphoma (DLBCL), GCB subtype. In some embodiments, the hematological malignancy is Waldenstrom's macroglobulinemia (WM). In some embodiments, the hematological malignancy is multiple myeloma (MM). In some embodiments, the hematological malignancy is Burkitt's lymphoma. In some embodiments, the hematological malignancy is follicular lymphoma (FL). In some embodiments, the hematological malignancy is transformed follicular lymphoma. In some embodiments, the hematological malignancy is marginal zone lymphoma.

In some embodiments, the hematological malignancy is relapsed or refractory non-Hodgkin's lymphoma (NHL). In some embodiments, the hematological malignancy is relapsed or refractory diffuse large B-cell lymphoma (DLBCL), relapsed or refractory mantle cell lymphoma (MCL), relapsed or refractory follicular lymphoma (FL), relapsed or refractory CLL, relapsed or refractory SLL, relapsed or refractory multiple myeloma, relapsed or refractory Waldenstrom's macroglobulinemia, relapsed or refractory multiple myeloma (MM), relapsed or refractory marginal zone lymphoma, relapsed or refractory Burkitt's lymphoma, relapsed or refractory non-Burkitt high grade B cell lymphoma, relapsed or refractory extranodal marginal zone B cell lymphoma. In some embodiments, the hematological malignancy is a relapsed or refractory acute or chronic myelogenous (or myeloid) leukemia, relapsed or refractory myelodysplastic syndrome, relapsed or refractory acute lymphoblastic leukemia, or relapsed or refractory precursor B-cell acute lymphoblastic leukemia. In some embodiments, the hematological malignancy is relapsed or refractory chronic lymphocytic leukemia (CLL). In some embodiments, the hematological malignancy is relapsed or refractory mantle cell lymphoma (MCL). In some embodiments, the hematological malignancy is relapsed or refractory diffuse large B-cell lymphoma (DLBCL). In some embodiments, the hematological malignancy is relapsed or refractory diffuse large B-cell lymphoma (DLBCL), ABC subtype. In some embodiments, the hematological malignancy is relapsed or refractory diffuse large B-cell lymphoma (DLBCL), GCB subtype. In some embodiments, the hematological malignancy is relapsed or refractory Waldenstrom's macroglobulinemia (WM). In some embodiments, the hematological malignancy is relapsed or refractory multiple myeloma (MM). In some embodiments, the hematological malignancy is relapsed or refractory Burkitt's lymphoma. In some embodiments, the hematological malignancy is relapsed or refractory follicular lymphoma (FL).

In some embodiments, the hematological malignancy is a hematological malignancy that is classified as high-risk. In some embodiments, the hematological malignancy is high risk CLL or high risk SLL.

In some embodiments, the hematologic malignancy is a T-cell malignancy. In some embodiments, the T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas. In some embodiments, the T-cell malignancy is a relapsed or refractory T-cell malignancy. In some embodiments, the T-cell malignancy is a treatment naïve T-cell malignancy.

B-cell lymphoproliferative disorders (BCLDs) are neoplasms of the blood and encompass, inter alia, non-Hodgkin lymphoma, multiple myeloma, and leukemia. BCLDs can originate either in the lymphatic tissues (as in the case of lymphoma) or in the bone marrow (as in the case of leukemia and myeloma), and they all are involved with the uncontrolled growth of lymphocytes or white blood cells. There are many subtypes of BCLD, e.g., chronic lymphocytic leukemia (CLL) and non-Hodgkin lymphoma (NHL). The disease course and treatment of BCLD is dependent on the BCLD subtype; however, even within each subtype the clinical presentation, morphologic appearance, and response to therapy is heterogeneous.

Malignant lymphomas are neoplastic transformations of cells that reside predominantly within lymphoid tissues. Two groups of malignant lymphomas are Hodgkin's lymphoma and non-Hodgkin's lymphoma (NHL). Both types of lymphomas infiltrate reticuloendothelial tissues. However, they differ in the neoplastic cell of origin, site of disease, presence of systemic symptoms, and response to treatment (Freedman et al., “Non-Hodgkin's Lymphomas” Chapter 134, Cancer Medicine, (an approved publication of the American Cancer Society, B.C. Decker Inc., Hamilton, Ontario, 2003).

Non-Hodgkin's Lymphomas

Disclosed herein, in certain embodiments, is a method for treating a non-Hodgkin's lymphoma in an individual in need thereof, comprising: administering to the individual an amount of a solvate of Compound 1.

Further disclosed herein, in certain embodiments, is a method for treating relapsed or refractory non-Hodgkin's lymphoma in an individual in need thereof, comprising: administering to the individual a therapeutically-effective amount of a solvate of Compound 1. In some embodiments, the non-Hodgkin's lymphoma is relapsed or refractory diffuse large B-cell lymphoma (DLBCL), relapsed or refractory mantle cell lymphoma, relapsed or refractory follicular lymphoma, or relapsed or refractory CLL.

Non-Hodgkin lymphomas (NHL) are a diverse group of malignancies that are predominately of B-cell origin. NHL may develop in any organs associated with lymphatic system such as spleen, lymph nodes or tonsils and can occur at any age. NHL is often marked by enlarged lymph nodes, fever, and weight loss. NHL is classified as either B-cell or T-cell NHL. Lymphomas related to lymphoproliferative disorders following bone marrow or stem cell transplantation are usually B-cell NHL. In the Working Formulation classification scheme, NHL has been divided into low-, intermediate-, and high-grade categories by virtue of their natural histories (see “The Non-Hodgkin's Lymphoma Pathologic Classification Project,” Cancer 49(1982):2112-2135). The low-grade lymphomas are indolent, with a median survival of 5 to 10 years (Horning and Rosenberg (1984) N. Engl. J. Med. 311:1471-1475). Although chemotherapy can induce remissions in the majority of indolent lymphomas, cures are rare and most patients eventually relapse, requiring further therapy. The intermediate- and high-grade lymphomas are more aggressive tumors, but they have a greater chance for cure with chemotherapy. However, a significant proportion of these patients will relapse and require further treatment.

A non-limiting list of the B-cell NHL includes Burkitt's lymphoma (e.g., Endemic Burkitt's Lymphoma and Sporadic Burkitt's Lymphoma), Cutaneous B-Cell Lymphoma, Cutaneous Marginal Zone Lymphoma (MZL), Diffuse Large Cell Lymphoma (DLBCL), Diffuse Mixed Small and Large Cell Lymphoma, Diffuse Small Cleaved Cell, Diffuse Small Lymphocytic Lymphoma, Extranodal Marginal Zone B-cell lymphoma, follicular lymphoma, Follicular Small Cleaved Cell (Grade 1), Follicular Mixed Small Cleaved and Large Cell (Grade 2), Follicular Large Cell (Grade 3), Intravascular Large B-Cell Lymphoma, Intravascular Lymphomatosis, Large Cell Immunoblastic Lymphoma, Large Cell Lymphoma (LCL), Lymphoblastic Lymphoma, MALT Lymphoma, Mantle Cell Lymphoma (MCL), immunoblastic large cell lymphoma, precursor B-lymphoblastic lymphoma, mantle cell lymphoma, chronic lymphocytic leukemia (CLL)/small lymphocytic lymphoma (SLL), extranodal marginal zone B-cell lymphoma-mucosa-associated lymphoid tissue (MALT) lymphoma, Mediastinal Large B-Cell Lymphoma, nodal marginal zone B-cell lymphoma, splenic marginal zone B-cell lymphoma, primary mediastinal B-cell lymphoma, lymphoplasmocytic lymphoma, hairy cell leukemia, Waldenstrom's Macroglobulinemia, and primary central nervous system (CNS) lymphoma. Additional non-Hodgkin's lymphomas are contemplated within the scope of the present invention and apparent to those of ordinary skill in the art.

DLBCL

Disclosed herein, in certain embodiments, is a method for treating a DLCBL in an individual in need thereof, comprising: administering to the individual an amount of a solvate of Compound 1. Further disclosed herein, in certain embodiments, is a method for treating relapsed or refractory DLCBL in an individual in need thereof, comprising: administering to the individual a therapeutically-effective amount of a solvate of Compound 1.

As used herein, the term “Diffuse large B-cell lymphoma (DLBCL)” refers to a neoplasm of the germinal center B lymphocytes with a diffuse growth pattern and a high-intermediate proliferation index. DLBCLs represent approximately 30% of all lymphomas and may present with several morphological variants including the centroblastic, immunoblastic, T-cell/histiocyte rich, anaplastic and plasmoblastic subtypes. Genetic tests have shown that there are different subtypes of DLBCL. In some embodiments, DLBCL is further divided into subtypes: activated B-cell diffuse large B-cell lymphoma (ABC-DLBCL), germinal center diffuse large B-cell lymphoma (GCB DLBCL), and Double-Hit (DH) DLBCL. In some embodiments, ABC-DLBCL is characterized by a CD79B mutation. In some embodiments, ABC-DLBCL is characterized by a CD79A mutation. In some embodiments, the ABC-DLBCL is characterized by a mutation in MyD88, A20, or a combination thereof. These subtypes seem to have different outlooks (prognoses) and responses to treatment. DLBCL can affect any age group but occurs mostly in older people (the average age is mid-60s).

Disclosed herein, in certain embodiments, is a method for treating diffuse large B-cell lymphoma, activated B cell-like subtype (ABC-DLBCL), in an individual in need thereof, comprising: administering to the individual an irreversible Btk inhibitor in an amount from 300 mg/day up to, and including, 1000 mg/day. The ABC subtype of diffuse large B-cell lymphoma (ABC-DLBCL) is thought to arise from post germinal center B cells that are arrested during plasmatic differentiation. The ABC subtype of DLBCL (ABC-DLBCL) accounts for approximately 30% total DLBCL diagnoses. It is considered the least curable of the DLBCL molecular subtypes and, as such, patients diagnosed with the ABC-DLBCL typically display significantly reduced survival rates compared with individuals with other types of DLCBL. ABC-DLBCL is most commonly associated with chromosomal translocations deregulating the germinal center master regulator BCL6 and with mutations inactivating the PRDM1 gene, which encodes a transcriptional repressor required for plasma cell differentiation.

A particularly relevant signaling pathway in the pathogenesis of ABC-DLBCL is the one mediated by the nuclear factor (NF)-κB transcription complex. The NF-κB family comprises 5 members (p50, p52, p65, c-rel and RelB) that form homo- and heterodimers and function as transcriptional factors to mediate a variety of proliferation, apoptosis, inflammatory and immune responses and are critical for normal B-cell development and survival. NF-κB is widely used by eukaryotic cells as a regulator of genes that control cell proliferation and cell survival. As such, many different types of human tumors have misregulated NF-κB: that is, NF-κB is constitutively active. Active NF-κB turns on the expression of genes that keep the cell proliferating and protect the cell from conditions that would otherwise cause it to die via apoptosis.

The dependence of ABC DLBCLs on NF-kB depends on a signaling pathway upstream of IkB kinase comprised of CARD11, BCL10 and MALT1 (the CBM complex). Interference with the CBM pathway extinguishes NF-kB signaling in ABC DLBCL cells and induces apoptosis. The molecular basis for constitutive activity of the NF-kB pathway is a subject of current investigation but some somatic alterations to the genome of ABC DLBCLs clearly invoke this pathway. For example, somatic mutations of the coiled-coil domain of CARD11 in DLBCL render this signaling scaffold protein able to spontaneously nucleate protein-protein interaction with MALT1 and BCL10, causing IKK activity and NF-kB activation. Constitutive activity of the B cell receptor signaling pathway has been implicated in the activation of NF-kB in ABC DLBCLs with wild type CARD11, and this is associated with mutations within the cytoplasmic tails of the B cell receptor subunits CD79A and CD79B. Oncogenic activating mutations in the signaling adapter MYD88 activate NF-kB and synergize with B cell receptor signaling in sustaining the survival of ABC DLBCL cells. In addition, inactivating mutations in a negative regulator of the NF-kB pathway, A20, occur almost exclusively in ABC DLBCL.

Indeed, genetic alterations affecting multiple components of the NF-κB signaling pathway have been recently identified in more than 50% of ABC-DLBCL patients, where these lesions promote constitutive NF-κB activation, thereby contributing to lymphoma growth. These include mutations of CARD11 (˜10% of the cases), a lymphocyte-specific cytoplasmic scaffolding protein that—together with MALT1 and BCL10—forms the BCR signalosome, which relays signals from antigen receptors to the downstream mediators of NF-κB activation. An even larger fraction of cases (˜30%) carry biallelic genetic lesions inactivating the negative NF-κB regulator A20. Further, high levels of expression of NF-κB target genes have been observed in ABC-DLBCL tumor samples. See, e.g., U. Klein et al., (2008), Nature Reviews Immunology 8:22-23; R. E. Davis et al., (2001), Journal of Experimental Medicine 194:1861-1874; G. Lentz et al., (2008), Science 319:1676-1679; M. Compagno et al., (2009), Nature 459:712-721; and L. Srinivasan et al., (2009), Cell 139:573-586).

DLBCL cells of the ABC subtype, such as OCI-Ly10, have chronic active BCR signaling and are very sensitive to the Btk inhibitor described herein. The irreversible Btk inhibitor described herein potently and irreversibly inhibits the growth of OCI-Ly10 (EC₅₀ continuous exposure=10 nM, EC₅₀ 1 hour pulse=50 nM). In addition, induction of apoptosis, as shown by capsase activation, Annexin-V flow cytometry and increase in sub-GO fraction is observed in OCILy10. Both sensitive and resistant cells express Btk at similar levels, and the active site of Btk is fully occupied by the inhibitor in both as shown using a fluorescently labeled affinity probe. OCI-Ly10 cells are shown to have chronically active BCR signaling to NF-kB which is dose dependently inhibited by the Btk inhibitors described herein. The activity of Btk inhibitors in the cell lines studied herein are also characterized by comparing signal transduction profiles (Btk, PLCγ, ERK, NF-kB, AKT), cytokine secretion profiles and mRNA expression profiles, both with and without BCR stimulation, and observed significant differences in these profiles that lead to clinical biomarkers that identify the most sensitive patient populations to Btk inhibitor treatment. See U.S. Pat. No. 7,711,492 and Staudt et al., Nature, Vol. 463, Jan. 7, 2010, pp. 88-92, the contents of which are incorporated by reference in their entirety.

Follicular Lymphoma

Disclosed herein, in certain embodiments, is a method for treating a follicular lymphoma in an individual in need thereof, comprising: administering to the individual an amount of a solvate of Compound 1. Further disclosed herein, in certain embodiments, is a method for treating relapsed or refractory follicular lymphoma in an individual in need thereof, comprising: administering to the individual a therapeutically-effective amount of a solvate of Compound 1.

As used herein, the term “follicular lymphoma” refers to any of several types of non-Hodgkin's lymphoma in which the lymphomatous cells are clustered into nodules or follicles. The term follicular is used because the cells tend to grow in a circular, or nodular, pattern in lymph nodes. The average age for people with this lymphoma is about 60.

CLL/SLL

Disclosed herein, in certain embodiments, is a method for treating a CLL or SLL in an individual in need thereof, comprising: administering to the individual an amount of a solvate of Compound 1. Further disclosed herein, in certain embodiments, is a method for treating relapsed or refractory CLL or SLL in an individual in need thereof, comprising: administering to the individual a therapeutically-effective amount of a solvate of Compound 1.

Chronic lymphocytic leukemia and small lymphocytic lymphoma (CLL/SLL) are commonly thought as the same disease with slightly different manifestations. Where the cancerous cells gather determines whether it is called CLL or SLL. When the cancer cells are primarily found in the lymph nodes, lima bean shaped structures of the lymphatic system (a system primarily of tiny vessels found in the body), it is called SLL. SLL accounts for about 5% to 10% of all lymphomas. When most of the cancer cells are in the bloodstream and the bone marrow, it is called CLL.

Both CLL and SLL are slow-growing diseases, although CLL, which is much more common, tends to grow slower. CLL and SLL are treated the same way. They are usually not considered curable with standard treatments, but depending on the stage and growth rate of the disease, most patients live longer than 10 years. Occasionally over time, these slow-growing lymphomas may transform into a more aggressive type of lymphoma.

Chronic lymphoid leukemia (CLL) is the most common type of leukemia. It is estimated that 100,760 people in the United States are living with or are in remission from CLL. Most (>75%) people newly diagnosed with CLL are over the age of 50. Currently CLL treatment focuses on controlling the disease and its symptoms rather than on an outright cure. CLL is treated by chemotherapy, radiation therapy, biological therapy, or bone marrow transplantation. Symptoms are sometimes treated surgically (splenectomy removal of enlarged spleen) or by radiation therapy (“de-bulking” swollen lymph nodes). Though CLL progresses slowly in most cases, it is considered generally incurable. Certain CLLs are classified as high-risk. As used herein, “high risk CLL” means CLL characterized by at least one of the following 1) 17p13−; 2) 11q22−; 3) unmutated IgVH together with ZAP-70+ and/or CD38+; or 4) trisomy 12.

CLL treatment is typically administered when the patient's clinical symptoms or blood counts indicate that the disease has progressed to a point where it may affect the patient's quality of life.

Small lymphocytic leukemia (SLL) is very similar to CLL described supra, and is also a cancer of B-cells. In SLL the abnormal lymphocytes mainly affect the lymph nodes. However, in CLL the abnormal cells mainly affect the blood and the bone marrow. The spleen may be affected in both conditions. SLL accounts for about 1 in 25 of all cases of non-Hodgkin lymphoma. It can occur at any time from young adulthood to old age, but is rare under the age of 50. SLL is considered an indolent lymphoma. This means that the disease progresses very slowly, and patients tend to live many years after diagnosis. However, most patients are diagnosed with advanced disease, and although SLL responds well to a variety of chemotherapy drugs, it is generally considered to be incurable. Although some cancers tend to occur more often in one gender or the other, cases and deaths due to SLL are evenly split between men and women. The average age at the time of diagnosis is 60 years.

Although SLL is indolent, it is persistently progressive. The usual pattern of this disease is one of high response rates to radiation therapy and/or chemotherapy, with a period of disease remission. This is followed months or years later by an inevitable relapse. Re-treatment leads to a response again, but again the disease will relapse. This means that although the short-term prognosis of SLL is quite good, over time, many patients develop fatal complications of recurrent disease. Considering the age of the individuals typically diagnosed with CLL and SLL, there is a need in the art for a simple and effective treatment of the disease with minimum side-effects that do not impede on the patient's quality of life. The instant invention fulfills this long standing need in the art.

Mantle Cell Lymphoma

Disclosed herein, in certain embodiments, is a method for treating a Mantle cell lymphoma in an individual in need thereof, comprising: administering to the individual an amount of a solvate of Compound 1. Further disclosed herein, in certain embodiments, is a method for treating relapsed or refractory Mantle cell lymphoma in an individual in need thereof, comprising: administering to the individual a therapeutically-effective amount of a solvate of Compound 1.

As used herein, the term, “Mantle cell lymphoma” refers to a subtype of B-cell lymphoma, due to CD5 positive antigen-naive pregerminal center B-cell within the mantle zone that surrounds normal germinal center follicles. MCL cells generally over-express cyclin D1 due to a t(11:14) chromosomal translocation in the DNA. More specifically, the translocation is at t(11;14)(q13;q32). Only about 5% of lymphomas are of this type. The cells are small to medium in size. Men are affected most often. The average age of patients is in the early 60s. The lymphoma is usually widespread when it is diagnosed, involving lymph nodes, bone marrow, and, very often, the spleen. Mantle cell lymphoma is not a very fast growing lymphoma, but is difficult to treat.

Marginal Zone B-Cell Lymphoma

Disclosed herein, in certain embodiments, is a method for treating a marginal zone B-cell lymphoma in an individual in need thereof, comprising: administering to the individual an amount of a solvate of Compound 1. Further disclosed herein, in certain embodiments, is a method for treating relapsed or refractory marginal zone B-cell lymphoma in an individual in need thereof, comprising: administering to the individual a therapeutically-effective amount of a solvate of Compound 1.

As used herein, the term “marginal zone B-cell lymphoma” refers to a group of related B-cell neoplasms that involve the lymphoid tissues in the marginal zone, the patchy area outside the follicular mantle zone. Marginal zone lymphomas account for about 5% to 10% of lymphomas. The cells in these lymphomas look small under the microscope. There are 3 main types of marginal zone lymphomas including extranodal marginal zone B-cell lymphomas, nodal marginal zone B-cell lymphoma, and splenic marginal zone lymphoma.

MALT

Disclosed herein, in certain embodiments, is a method for treating a MALT in an individual in need thereof, comprising: administering to the individual an amount of a solvate of Compound 1. Further disclosed herein, in certain embodiments, is a method for treating relapsed or refractory MALT in an individual in need thereof, comprising: administering to the individual a therapeutically-effective amount of a solvate of Compound 1.

The term “mucosa-associated lymphoid tissue (MALT) lymphoma”, as used herein, refers to extranodal manifestations of marginal-zone lymphomas. Most MALT lymphoma are a low grade, although a minority either manifest initially as intermediate-grade non-Hodgkin lymphoma (NHL) or evolve from the low-grade form. Most of the MALT lymphoma occur in the stomach, and roughly 70% of gastric MALT lymphoma are associated with Helicobacter pylori infection. Several cytogenetic abnormalities have been identified, the most common being trisomy 3 or t(11;18). Many of these other MALT lymphoma have also been linked to infections with bacteria or viruses. The average age of patients with MALT lymphoma is about 60.

Nodal Marginal Zone B-Cell Lymphoma

Disclosed herein, in certain embodiments, is a method for treating a nodal marginal zone B-cell lymphoma in an individual in need thereof, comprising: administering to the individual an amount of a solvate of Compound 1. Further disclosed herein, in certain embodiments, is a method for treating relapsed or refractory nodal marginal zone B-cell lymphoma in an individual in need thereof, comprising: administering to the individual a therapeutically-effective amount of a solvate of Compound 1.

The term “nodal marginal zone B-cell lymphoma” refers to an indolent B-cell lymphoma that is found mostly in the lymph nodes. The disease is rare and only accounts for 1% of all Non-Hodgkin's Lymphomas (NHL). It is most commonly diagnosed in older patients, with women more susceptible than men. The disease is classified as a marginal zone lymphoma because the mutation occurs in the marginal zone of the B-cells. Due to its confinement in the lymph nodes, this disease is also classified as nodal.

Splenic Marginal Zone B-Cell Lymphoma

Disclosed herein, in certain embodiments, is a method for treating a splenic marginal zone B-cell lymphoma in an individual in need thereof, comprising: administering to the individual an amount of a solvate of Compound 1. Further disclosed herein, in certain embodiments, is a method for treating relapsed or refractory splenic marginal zone B-cell lymphoma in an individual in need thereof, comprising: administering to the individual a therapeutically-effective amount of a solvate of Compound 1.

The term “splenic marginal zone B-cell lymphoma” refers to specific low-grade small B-cell lymphoma that is incorporated in the World Health Organization classification. Characteristic features are splenomegaly, moderate lymphocytosis with villous morphology, intrasinusoidal pattern of involvement of various organs, especially bone marrow, and relative indolent course. Tumor progression with increase of blastic forms and aggressive behavior are observed in a minority of patients. Molecular and cytogenetic studies have shown heterogeneous results probably because of the lack of standardized diagnostic criteria.

Burkitt Lymphoma

Disclosed herein, in certain embodiments, is a method for treating a Burkitt lymphoma in an individual in need thereof, comprising: administering to the individual an amount of a solvate of Compound 1. Further disclosed herein, in certain embodiments, is a method for treating relapsed or refractory Burkitt lymphoma in an individual in need thereof, comprising: administering to the individual a therapeutically-effective amount of a solvate of Compound 1.

The term “Burkitt lymphoma” refers to a type of Non-Hodgkin Lymphoma (NHL) that commonly affects children. It is a highly aggressive type of B-cell lymphoma that often starts and involves body parts other than lymph nodes. In spite of its fast-growing nature, Burkitt's lymphoma is often curable with modern intensive therapies. There are two broad types of Burkitt's lymphoma—the sporadic and the endemic varieties:

Endemic Burkitt's lymphoma: The disease involves children much more than adults, and is related to Epstein Barr Virus (EBV) infection in 95% cases. It occurs primarily is equatorial Africa, where about half of all childhood cancers are Burkitt's lymphoma. It characteristically has a high chance of involving the jawbone, a rather distinctive feature that is rare in sporadic Burkitt's. It also commonly involves the abdomen.

Sporadic Burkitt's lymphoma: The type of Burkitt's lymphoma that affects the rest of the world, including Europe and the Americas is the sporadic type. Here too, it's mainly a disease in children. The link between Epstein Barr Virus (EBV) is not as strong as with the endemic variety, though direct evidence of EBV infection is present in one out of five patients. More than the involvement of lymph nodes, it is the abdomen that is notably affected in more than 90% of the children. Bone marrow involvement is more common than in the sporadic variety.

Waldenstrom Macroglobulinemia

Disclosed herein, in certain embodiments, is a method for treating a Waldenstrom macroglobulinemia in an individual in need thereof, comprising: administering to the individual an amount of a solvate of Compound 1. Further disclosed herein, in certain embodiments, is a method for treating relapsed or refractory Waldenstrom macroglobulinemia in an individual in need thereof, comprising: administering to the individual a therapeutically-effective amount of a solvate of Compound 1.

The term “Waldenstrom macroglobulinemia”, also known as lymphoplasmacytic lymphoma, is cancer involving a subtype of white blood cells called lymphocytes. It is characterized by an uncontrolled clonal proliferation of terminally differentiated B lymphocytes. It is also characterized by the lymphoma cells making an antibody called immunoglobulin M (IgM). The IgM antibodies circulate in the blood in large amounts, and cause the liquid part of the blood to thicken, like syrup. This can lead to decreased blood flow to many organs, which can cause problems with vision (because of poor circulation in blood vessels in the back of the eyes) and neurological problems (such as headache, dizziness, and confusion) caused by poor blood flow within the brain. Other symptoms can include feeling tired and weak, and a tendency to bleed easily. The underlying etiology is not fully understood but a number of risk factors have been identified, including the locus 6p21.3 on chromosome 6. There is a 2- to 3-fold risk increase of developing WM in people with a personal history of autoimmune diseases with autoantibodies and particularly elevated risks associated with hepatitis, human immunodeficiency virus, and rickettsiosis.

Multiple Myeloma

Disclosed herein, in certain embodiments, is a method for treating a myeloma in an individual in need thereof, comprising: administering to the individual an amount of a solvate of Compound 1. Further disclosed herein, in certain embodiments, is a method for treating relapsed or refractory myeloma in an individual in need thereof, comprising: administering to the individual a therapeutically-effective amount of a solvate of Compound 1.

Multiple myeloma, also known as MM, myeloma, plasma cell myeloma, or as Kahler's disease (after Otto Kahler) is a cancer of the white blood cells known as plasma cells. A type of B cell, plasma cells are a crucial part of the immune system responsible for the production of antibodies in humans and other vertebrates. They are produced in the bone marrow and are transported through the lymphatic system.

Leukemia

Disclosed herein, in certain embodiments, is a method for treating a leukemia in an individual in need thereof, comprising: administering to the individual an amount of a solvate of Compound 1. Further disclosed herein, in certain embodiments, is a method for treating relapsed or refractory leukemia in an individual in need thereof, comprising: administering to the individual a therapeutically-effective amount of a solvate of Compound 1.

Leukemia is a cancer of the blood or bone marrow characterized by an abnormal increase of blood cells, usually leukocytes (white blood cells). Leukemia is a broad term covering a spectrum of diseases. The first division is between its acute and chronic forms: (i) acute leukemia is characterized by the rapid increase of immature blood cells. This crowding makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children; (ii) chronic leukemia is distinguished by the excessive build up of relatively mature, but still abnormal, white blood cells. Typically taking months or years to progress, the cells are produced at a much higher rate than normal cells, resulting in many abnormal white blood cells in the blood. Chronic leukemia mostly occurs in older people, but can theoretically occur in any age group. Additionally, the diseases are subdivided according to which kind of blood cell is affected. This split divides leukemias into lymphoblastic or lymphocytic leukemias and myeloid or myelogenous leukemias: (i) lymphoblastic or lymphocytic leukemias, the cancerous change takes place in a type of marrow cell that normally goes on to form lymphocytes, which are infection-fighting immune system cells; (ii) myeloid or myelogenous leukemias, the cancerous change takes place in a type of marrow cell that normally goes on to form red blood cells, some other types of white cells, and platelets.

Within these main categories, there are several subcategories including, but not limited to, Acute lymphoblastic leukemia (ALL), precursor B-cell acute lymphoblastic leukemia (precursor B-ALL; also called precursor B-lymphoblastic leukemia), Acute myelogenous leukemia (AML), Chronic myelogenous leukemia (CML), and Hairy cell leukemia (HCL). Accordingly, disclosed herein, in certain embodiments, is a method for treating Acute lymphoblastic leukemia (ALL), precursor B-cell acute lymphoblastic leukemia (precursor B-ALL; also called precursor B-lymphoblastic leukemia), Acute myelogenous leukemia (AML), Chronic myelogenous leukemia (CML), or Hairy cell leukemia (HCL) in an individual in need thereof, comprising: administering to the individual an amount of a solvate of Compound 1. In some embodiments, the leukemia is a relapsed or refractory leukemia. In some embodiments, the leukemia is a relapsed or refractory Acute lymphoblastic leukemia (ALL), relapsed or refractory precursor B-cell acute lymphoblastic leukemia (precursor B-ALL; also called precursor B-lymphoblastic leukemia), relapsed or refractory Acute myelogenous leukemia (AML), relapsed or refractory Chronic myelogenous leukemia (CML), or relapsed or refractory Hairy cell leukemia (HCL).

Symptoms, diagnostic tests, and prognostic tests for each of the above-mentioned conditions are known. See, e.g., Harrison's Principles of Internal Medicine©,” 16th ed., 2004, The McGraw-Hill Companies, Inc. Dey et al. (2006), Cytojournal 3(24), and the “Revised European American Lymphoma” (REAL) classification system (see, e.g., the website maintained by the National Cancer Institute).

A number of animal models of are useful for establishing a range of therapeutically effective doses of irreversible Btk inhibitor compounds, such as a solvate of Compound 1, for treating any of the foregoing diseases.

The therapeutic efficacy of a solvate of Compound 1 for any one of the foregoing diseases can be optimized during a course of treatment. For example, a subject being treated can undergo a diagnostic evaluation to correlate the relief of disease symptoms or pathologies to inhibition of in vivo Btk activity achieved by administering a given dose of Compound 1 in a solvate form. Cellular assays known in the art can be used to determine in vivo activity of Btk in the presence or absence of an irreversible Btk inhibitor. For example, since activated Btk is phosphorylated at tyrosine 223 (Y223) and tyrosine 551 (Y551), phospho-specific immunocytochemical staining of P-Y223 or P-Y551-positive cells can be used to detect or quantify activation of Btk in a population of cells (e.g., by FACS analysis of stained vs unstained cells). See, e.g., Nisitani et al. (1999), Proc. Natl. Acad. Sci. USA 96:2221-2226. Thus, the amount of the Btk inhibitor compound that is administered to a subject can be increased or decreased as needed so as to maintain a level of Btk inhibition optimal for treating the subject's disease state.

Compound 1 can irreversibly inhibit Btk and may be used to treat mammals suffering from Bruton's tyrosine kinase-dependent or Bruton's tyrosine kinase mediated conditions or diseases, including, but not limited to, cancer, autoimmune and other inflammatory diseases. Compound 1 has shown efficacy in a wide variety of diseases and conditions that are described herein.

In some embodiments, a solvate of Compound 1 is used for the manufacture of a medicament for treating any of the foregoing conditions (e.g., autoimmune diseases, inflammatory diseases, allergy disorders, B-cell proliferative disorders, or thromboembolic disorders).

In some embodiments, a solvate of Compound 1 described herein can be used to treat a solid tumor. In some embodiments, a solvate of Compound 1 described herein can be used to treat carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, or Smoldering of indolent multiple myeloma.

In some embodiments, the composition is for use in treatment of a sarcoma or carcinoma. In some embodiments, the composition is for use in treatment of a sarcoma. In some embodiments, the composition is for use in treatment of a carcinoma. In some embodiments, the sarcoma is selected from alveolar rhabdomyosarcoma; alveolar soft part sarcoma; ameloblastoma; angiosarcoma; chondrosarcoma; chordoma; clear cell sarcoma of soft tissue; dedifferentiated liposarcoma; desmoid; desmoplastic small round cell tumor; embryonal rhabdomyosarcoma; epithelioid fibrosarcoma; epithelioid hemangioendothelioma; epithelioid sarcoma; esthesioneuroblastoma; Ewing sarcoma; extrarenal rhabdoid tumor; extraskeletal myxoid chondrosarcoma; extrasketetal osteosarcoma; fibrosarcoma; giant cell tumor; hemangiopericytoma; infantile fibrosarcoma; inflammatory myofibroblastic tumor; Kaposi sarcoma; leiomyosarcoma of bone; liposarcoma; liposarcoma of bone; malignant fibrous histiocytoma (MFH); malignant fibrous histiocytoma (MFH) of bone; malignant mesenchymoma; malignant peripheral nerve sheath tumor; mesenchymal chondrosarcoma; myxofibrosarcoma; myxoid liposarcoma; myxoinflammatory fibroblastic sarcoma; neoplasms with perivascular epitheioid cell differentiation; osteosarcoma; parosteal osteosarcoma; neoplasm with perivascular epitheioid cell differentiation; periosteal osteosarcoma; pleomorphic liposarcoma; pleomorphic rhabdomyosarcoma; PNET/extraskeletal Ewing tumor; rhabdomyosarcoma; round cell liposarcoma; small cell osteosarcoma; solitary fibrous tumor; synovial sarcoma; telangiectatic osteosarcoma. In some embodiments, the carcinoma is selected from an adenocarcinoma, squamous cell carcinoma, adenosquamous carcinoma, anaplastic carcinoma, large cell carcinoma, or small cell carcinoma. In some embodiments, the solid tumor is selected from anal cancer; appendix cancer; bile duct cancer (i.e., cholangiocarcinoma); bladder cancer; brain tumor; breast cancer; HER2-amplified breast cancer; cervical cancer; colon cancer; cancer of Unknown Primary (CUP); esophageal cancer; eye cancer; fallopian tube cancer; kidney cancer; renal cell carcinoma; liver cancer; lung cancer; medulloblastoma; melanoma; oral cancer; ovarian cancer; pancreatic cancer; pancreatic ductal cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments, the carcinoma is breast cancer. In some embodiments, the breast cancer is invasive ductal carcinoma, ductal carcinoma in situ, invasive lobular carcinoma, or lobular carcinoma in situ. In some embodiments, the carcinoma is pancreatic cancer. In some embodiments, the pancreatic cancer is adenocarcinoma, or islet cell carcinoma. In some embodiments, the carcinoma is colorectal cancer. In some embodiments, the colorectal cancer is adenocarcinoma. In some embodiments, the solid tumor is a colon polyp. In some embodiments, the colon polyp is associated with familial adenomatous polyposis. In some embodiments, the carcinoma is bladder cancer. In some embodiments, the bladder cancer is transitional cell bladder cancer, squamous cell bladder cancer, or adenocarcinoma. In some embodiments, the carcinoma is lung cancer. In some embodiments, the lung cancer is a non-small cell lung cancer. In some embodiments, the non-small cell lung cancer is adenocarcinoma, squamous-cell lung carcinoma, or large-cell lung carcinoma. In some embodiments, the non-small cell lung cancer is large cel lung cancer. In some embodiments, the lung cancer is a small cell lung cancer. In some embodiments, the carcinoma is prostate cancer. In some embodiments, the prostate cancer is adenocarcinoma or small cell carcinoma. In some embodiments, the carcinoma is ovarian cancer. In some embodiments, the ovarian cancer is epithelial ovarian cancer. In some embodiments, the carcinoma is bile duct cancer. In some embodiments, the bile duct cancer is proximal bile duct carcinoma or distal bile duct carcinoma.

In some embodiments, the composition and methods described herein can be used to treat mastocytosis.

In some embodiments, a solvate of Compound 1 described herein can be used to treat a central nervous system (CNS) malignancy. In some embodiments, the CNS malignancy is a primary CNS lymphoma. In some embodiments the primary CNS lymphoma is a glioma. In some embodiments the glioma is astrocytomas, ependymomas, oligodendrogliomas. In some embodiments the CNS malignancy is astrocytic tumors such as juvenile pilocytic, subependymal, well differentiated or moderately differentiated anaplastic astrocytoma; anaplastic astrocytoma; glioblastoma multiforme; ependymal tumors such as myxopapillary and well-differentiated ependymoma, anaplastic ependymoma, ependymoblastoma; oligodendroglial tumors including well-differentiated oligodendroglioma and anaplastic oligodendroglioma; mixed tumors such as mixed astrocytoma-ependymoma, mixed astrocytoma-oligodendroglioma, mixed astrocytomaependymoma-oligodendroglioma; or medulloblastoma.

In some embodiments, a solvate of Compound 1 described herein can be used to treat fibrosis. In some embodiments, the fibrosis is not associated with graft versus host disease (GVHD). In some embodiments, the fibrosis is not associated with sclerodermatous GVHD, lung chronic GVHD, or liver chronic GVHD. In some embodiments, the fibrosis is of the liver, lung, pancreas, kidney, bone marrow, heart, skin, intestine, or joints. In some embodiments, the fibrosis is of the liver. In some embodiments, the fibrosis is of the lung. In some embodiments, the fibrosis is of the pancreas. In some embodiments, the patient has cirrhosis, chronic pancreatitis, or cystic fibrosis.

### Compound 1, and Pharmaceutically Acceptable Salts Thereof

The Btk inhibitor compound described herein (i.e. Compound 1) is selective for Btk and kinases having a cysteine residue in an amino acid sequence position of the tyrosine kinase that is homologous to the amino acid sequence position of cysteine 481 in Btk. The Btk inhibitor compound can form a covalent bond with Cys 481 of Btk (e.g., via a Michael reaction).

“Compound 1” or “1-((R)-3-(4-amino-3-(4-phenoxyphenyl)-1H-pyrazolo[3,4-d]pyrimidin-1-yl)piperidin-1-yl)prop-2-en-1-one” or “1-{(3R)-3-[4-amino-3-(4-phenoxyphenyl)-1H-pyrazolo[3,4-d]pyrimidin-1-yl]piperidin-1-yl}prop-2-en-1-one” or “2-Propen-1-one, 1-[(3R)-3-[4-amino-3-(4-phenoxyphenyl)-1H-pyrazolo[3,4-d]pyrimidin-1-yl]-1-piperidinyl-” or ibrutinib or any other suitable name refers to the compound with the following structure:

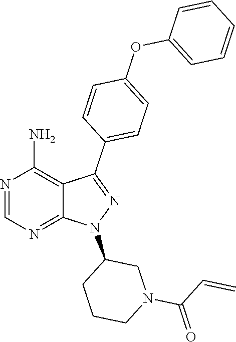 [SMILES: C=CC(=O)N1CCC[C@@H](n2nc(-c3ccc(Oc4ccccc4)cc3)c3c(N)ncnc32)C1]

A wide variety of pharmaceutically acceptable salts is formed from Compound 1 and includes:
acid addition salts formed by reacting Compound 1 with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, critric acid, benzoic acid, cinnamic acid, mandelic acid, methanesulfonic acid, ethanesulfonica acid, p-toluenesulfonic acid, salicylic acid, and the like;
acid addition salts formed by reacting Compound 1 with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to Compound 1 refers to a salt of Compound 1, which does not cause significant irritation to a mammal to which it is administered and does not substantially abrogate the biological activity and properties of the compound.

Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, acetophenone, benzyl acetate, butyronitrile, chlorobenzene, 1,2-dimethoxyethane, dimethylacetamide, hexafluorobenzene, 1,1,2-trichloroethane, and the like. In one aspect, solvates are formed using, but not limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of Compound 1, or pharmaceutically acceptable salts thereof, are prepared or formed by the processes described herein. In some embodiments, solvates of Compound 1 are anhydrous. It should be understood that a reference to a pharmaceutically acceptable salt includes the solvent addition forms (solvates).

In yet other embodiments, a solvate of Compound 1, or a pharmaceutically acceptable salt thereof, is prepared in various forms, including but not limited to, amorphous phase, crystalline forms, milled forms and nano-particulate forms. In some embodiments, a solvate of Compound 1, or a pharmaceutically acceptable salt thereof, is amorphous. In some embodiments, a solvate of Compound 1, or a pharmaceutically acceptable salt thereof, is amorphous and anhydrous. In some embodiments, a solvate of Compound 1, or a pharmaceutically acceptable salt thereof, is crystalline. In some embodiments, a solvate of Compound 1, or a pharmaceutically acceptable salt thereof, is crystalline and anhydrous. In some embodiments, a solvate of Compound 1, or a pharmaceutically acceptable salt thereof, is comtemplated to provide improved solubility and/or bioavailability. In some embodiments, a solvate of Compound 1, or a pharmaceutically acceptable salt thereof, is stable. In some embodiments, a solvate of Compound 1, or a pharmaceutically acceptable salt thereof, converts to a more stable crystalline form of Compound 1, or a pharmaceutically acceptable salt or solvate thereof, and may be useful in the preparation, such as purification, of Compound 1 or the more stable crystalline form of Compound 1, or a pharmaceutically acceptable salt or solvate thereof.

In some embodiments, Compound 1 is prepared as outlined in U.S. Pat. No. 7,514,444 (incorporated by reference).

### Butyronitrile Solvate of Compound 1, Crystalline Form 1

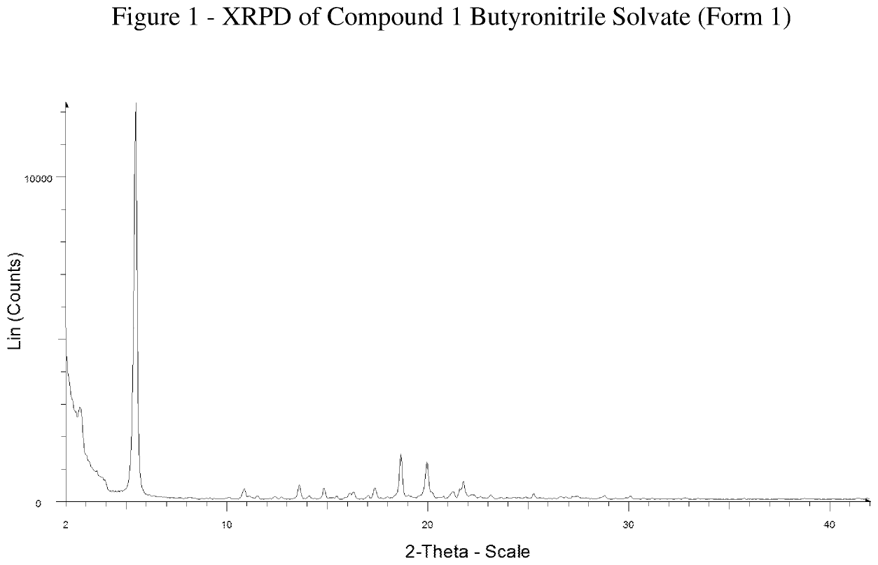

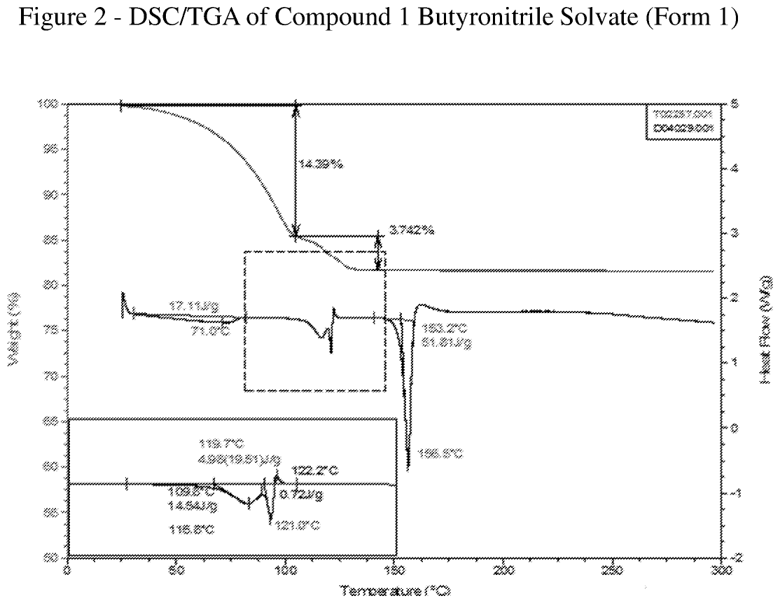

In some embodiments, Compound 1 is a butyronitrile solvate. In some embodiments, the butyronitrile solvate of Compound 1 is crystalline Form 1. The crystalline form of the butyronitrile solvate of Compound 1 (Form 1) is characterized as having at least one of the following properties:
(a) an X-ray powder diffraction (XRPD) pattern substantially the same as shown in FIG. 1;
(b) an X-ray powder diffraction (XRPD) pattern with at least two of the characteristic peaks at 2.7±0.1° 2-Theta, 5.5±0.1° 2-Theta, 10.9±0.1° 2-Theta, 13.6±0.1° 2-Theta, 14.8±0.1 2-Theta, 17.3±0.1° 2-Theta, 18.7±0.1° 2-Theta, 20.0±0.1° 2-Theta, and 21.8±0.10 2-Theta;
(c) a DSC thermogram substantially the same as the one set forth in FIG. 2;
(d) a DSC thermogram with an endotherm event at between about 100-125° C.;
(e) a thermo-gravimetric analysis (TGA) thermogram substantially the same as the one set forth in FIG. 2;
or
(f) combinations thereof.

In some embodiments, the crystalline form of the butyronitrile solvate of Compound 1 (Form 1) is characterized as having at least two of the properties selected from (a) to (e). In some embodiments, the crystalline form of the butyronitrile solvate of Compound 1 (Form 1) is characterized as having at least three of the properties selected from (a) to (e). In some embodiments, the crystalline form of the butyronitrile solvate of Compound 1 (Form 1) is characterized as having at least four of the properties selected from (a) to (e). In some embodiments, the crystalline form of the butyronitrile solvate of Compound 1 (Form 1) is characterized as having properties (a) to (e).

In some embodiments, the crystalline form of the butyronitrile solvate of Compound 1 (Form 1) has an X-ray powder diffraction (XRPD) pattern substantially the same as shown in FIG. 1. In some embodiments, the crystalline form of the butyronitrile solvate of Compound 1 (Form 1) has an X-ray powder diffraction (XRPD) pattern with characteristic peaks at 2.7±0.1° 2-Theta, 5.5±0.1° 2-Theta, 10.9±0.1° 2-Theta, 13.6±0.1° 2-Theta, 14.8±0.1° 2-Theta, 17.3±0.1° 2-Theta, 18.7±0.1° 2-Theta, 20.0±0.1° 2-Theta, and 21.8±0.1° 2-Theta.

In some embodiments, the crystalline form of the butyronitrile solvate of Compound 1 (Form 1) has a DSC thermogram substantially the same as the one set forth in FIG. 2. In some embodiments, the crystalline form of the butyronitrile solvate of Compound 1 (Form 1) has a DSC thermogram with an endotherm at between about 100-125° C. In some embodiments, the endotherm event have an onset at about 110° C., a first peak at about 120° C. and a second peak at about 121° C. In some embodiments, the DSC thermogram further has an endotherm having an onset at about 153° C. and a peak at about 156° C. In some embodiments, the crystalline form of the butyronitrile solvate of Compound 1 (Form 1) has a thermo-gravimetric analysis (TGA) thermogram substantially the same as the one set forth in FIG. 2.

### 1,2-Dimethoxyethane Solvate of Compound 1, Crystalline Form 2

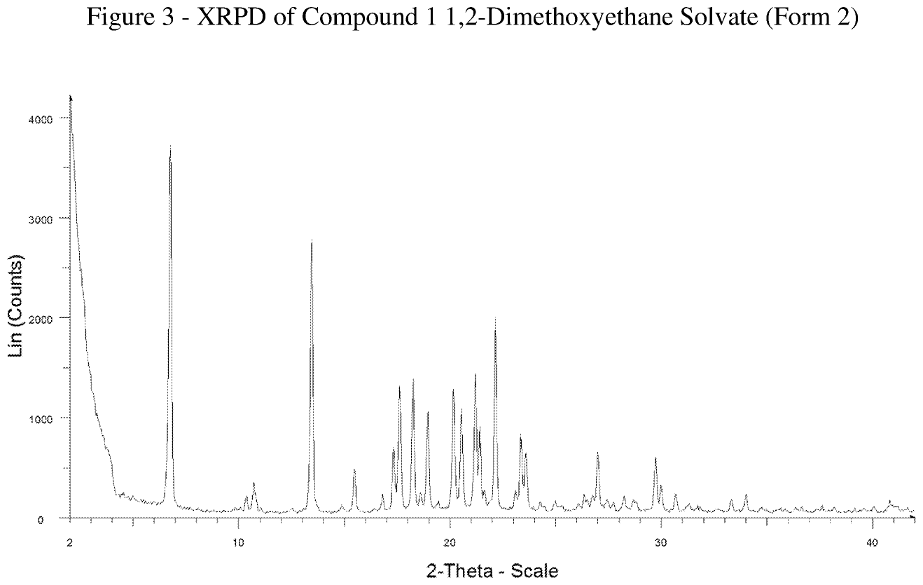

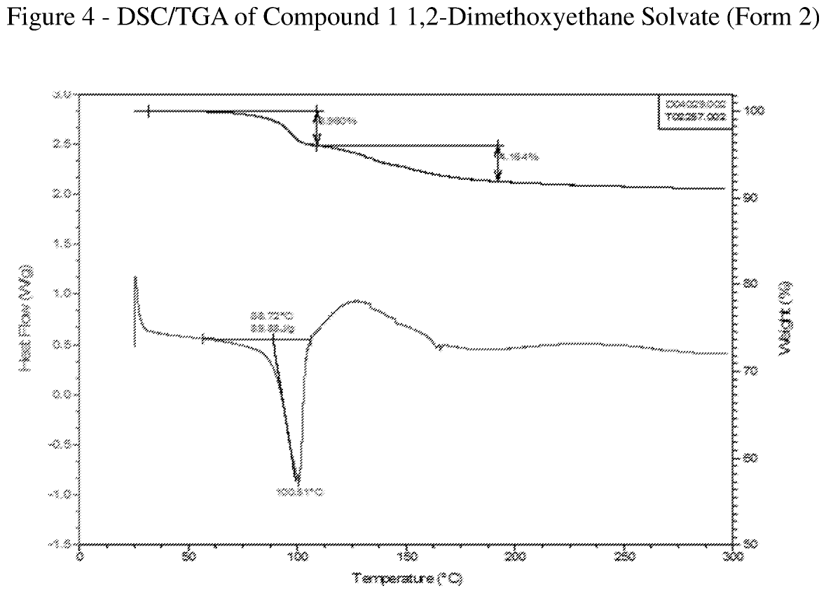

In some embodiments, provided is a Compound 1 1,2-dimethoxyethane solvate. In some embodiments, the 1,2-dimethoxyethane solvate of Compound 1 is crystalline Form 2. The crystalline form of the 1,2-dimethoxyethane solvate of Compound 1 (Form 2) is characterized as having at least one of the following properties:
(a) an X-ray powder diffraction (XRPD) pattern substantially the same as shown in FIG. 3;
(b) an X-ray powder diffraction (XRPD) pattern with at least two of the characteristic peaks at 6.8±0.1° 2-Theta, 13.4±0.1° 2-Theta, 17.6±0.1° 2-Theta, 18.2±0.1° 2-Theta, 20.2±0.1° 2-Theta, 21.2±0.1° 2-Theta, and 22.2±0.1° 2-Theta;
(c) substantially the same X-ray powder diffraction (XRPD) pattern post storage at 40° C. and 75% RH for at least a week;
(d) a DSC thermogram substantially the same as the one set forth in FIG. 4;
(e) a DSC thermogram with an endotherm having an onset at about 89° C. and a peak at about 101° C.;
(f) a thermo-gravimetric analysis (TGA) thermogram substantially the same as the one set forth in FIG. 4;
   or
(g) combinations thereof.

In some embodiments, the crystalline form of the 1,2-dimethoxyethane solvate of Compound 1 (Form 2) is characterized as having at least two of the properties selected from (a) to (f). In some embodiments, the crystalline form of the 1,2-dimethoxyethane solvate of Compound 1 (Form 2) is characterized as having at least three of the properties selected from (a) to (f). In some embodiments, the crystalline form of the 1,2-dimethoxyethane solvate of Compound 1 (Form 2) is characterized as having at least four of the properties selected from (a) to (f). In some embodiments, the crystalline form of the 1,2-dimethoxyethane solvate of Compound 1 (Form 2) is characterized as having at least five of the properties selected from (a) to (f). In some embodiments, the crystalline form of the 1,2-dimethoxyethane solvate of Compound 1 (Form 2) is characterized as having properties (a) to (f).

In some embodiments, the crystalline form of the 1,2-dimethoxyethane solvate of Compound 1 (Form 2) has an X-Ray powder diffraction (XRPD) pattern substantially the same as shown in FIG. 3. In some embodiments, the crystalline form of the 1,2-dimethoxyethane solvate of Compound 1 (Form 2) has an X-ray powder diffraction (XRPD) pattern with characteristic peaks at 6.8±0.1° 2-Theta, 13.4±0.1° 2-Theta, 17.6±0.1° 2-Theta, 18.2±0.1° 2-Theta, 20.2±0.1° 2-Theta, 21.2±0.1° 2-Theta, and 22.2±0.1° 2-Theta. In some embodiments, the crystalline form of the 1,2-dimethoxyethane solvate of Compound 1 (Form 2) has substantially the same X-ray powder diffraction (XRPD) pattern post storage at 40° C. and 75% RH for at least a week.

In some embodiments, the crystalline form of the 1,2-dimethoxyethane solvate of Compound 1 (Form 2) has a DSC thermogram substantially the same as the one set forth in FIG. 4. In some embodiments, the crystalline form of the 1,2-dimethoxyethane solvate of Compound 1 (Form 2) has a DSC thermogram with an endotherm having an onset at about 89° C. and a peak at about 101° C. In some embodiments, the crystalline form of the 1,2-dimethoxyethane solvate of Compound 1 (Form 2) has a thermo-gravimetric analysis (TGA) thermogram substantially the same as the one set forth in FIG. 4.

### Hexafluorobenzene Solvate of Compound 1, Crystalline Form 3

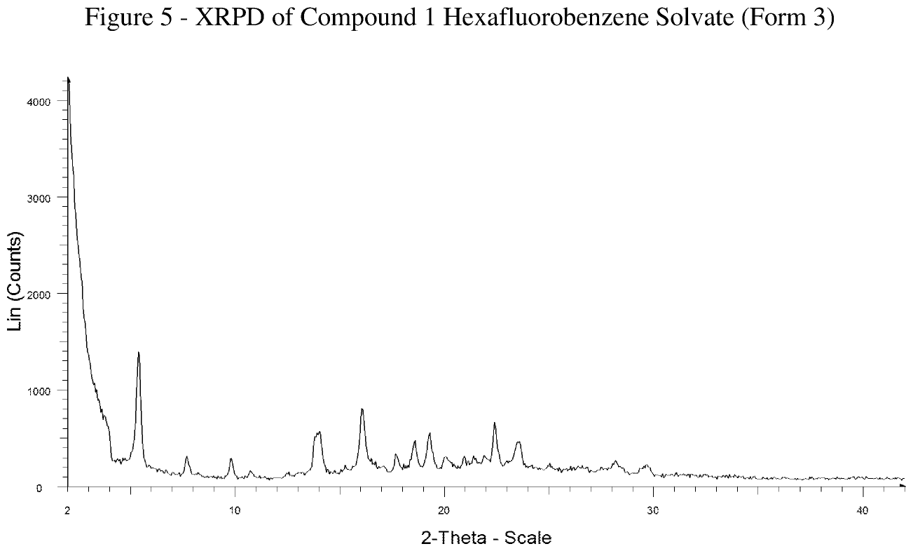

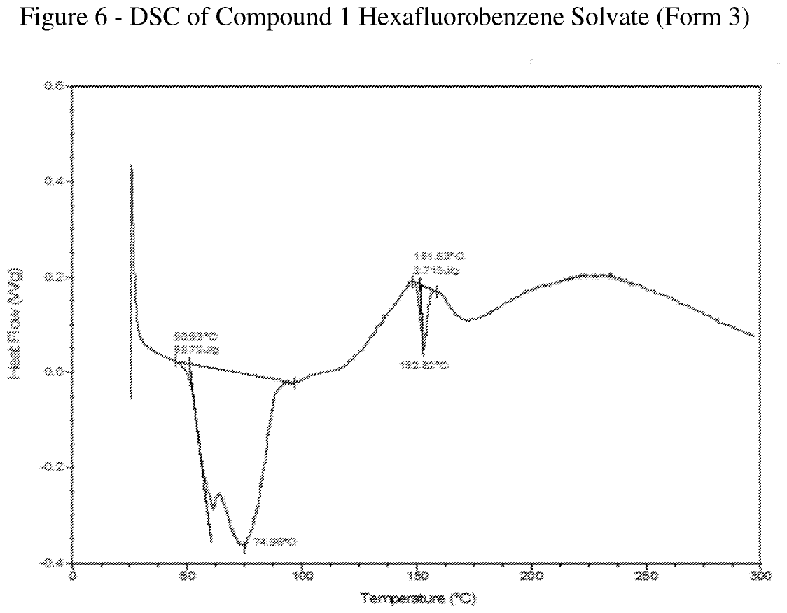

In some embodiments, provided is a Compound 1 hexafluorobenzene solvate. In some embodiments, the hexafluorobenzene solvate of Compound 1 is crystalline Form 3. The crystalline form of the hexafluorobenzene solvate of Compound 1 (Form 3) is characterized as having at least one of the following properties:
(a) an X-ray powder diffraction (XRPD) pattern substantially the same as shown in FIG. 5;
(b) an X-ray powder diffraction (XRPD) pattern with at least two of the characteristic peaks at 5.4±0.1° 2-Theta, 14.0±0.1° 2-Theta, 16.1±0.1° 2-Theta, 18.6±0.1° 2-Theta, 19.3±0.1° 2-Theta, 22.4±0.1° 2-Theta, and 23.6±0.1° 2-Theta;
(c) a DSC thermogram substantially the same as the one set forth in FIG. 6;
(d) a DSC thermogram with an endotherm having an onset at about 51° C.;
   or
(e) combinations thereof.

In some embodiments, the crystalline form of the hexafluorobenzene solvate of Compound 1 (Form 3) is characterized as having at least two of the properties selected from (a) to (d). In some embodiments, the crystalline form of the hexafluorobenzene solvate of Compound 1 (Form 3) is characterized as having at least three of the properties selected from (a) to (d). In some embodiments, the crystalline form of the hexafluorobenzene solvate of Compound 1 (Form 3) is characterized as having properties (a) to (d).

In some embodiments, the crystalline form of the hexafluorobenzene solvate of Compound 1 (Form 3) has an X-Ray powder diffraction (XRPD) pattern substantially the same as shown in FIG. 5. In some embodiments, the crystalline form of the hexafluorobenzene solvate of Compound 1 (Form 3) has an X-ray powder diffraction (XRPD) pattern with characteristic peaks at 5.4±0.1° 2-Theta, 14.0±0.1° 2-Theta, 16.1±0.1° 2-Theta, 18.6±0.1° 2-Theta, 19.3±0.1° 2-Theta, 22.4±0.1° 2-Theta, and 23.6±0.1° 2-Theta.

In some embodiments, the crystalline form of the hexafluorobenzene solvate of Compound 1 (Form 3) has a DSC thermogram substantially the same as the one set forth in FIG. 6. In some embodiments, the crystalline form of the hexafluorobenzene solvate of Compound 1 (Form 3) has a DSC thermogram with an endotherm having an onset at about 51° C.

### Hexafluorobenzene Solvate of Compound 1, Crystalline Form 4

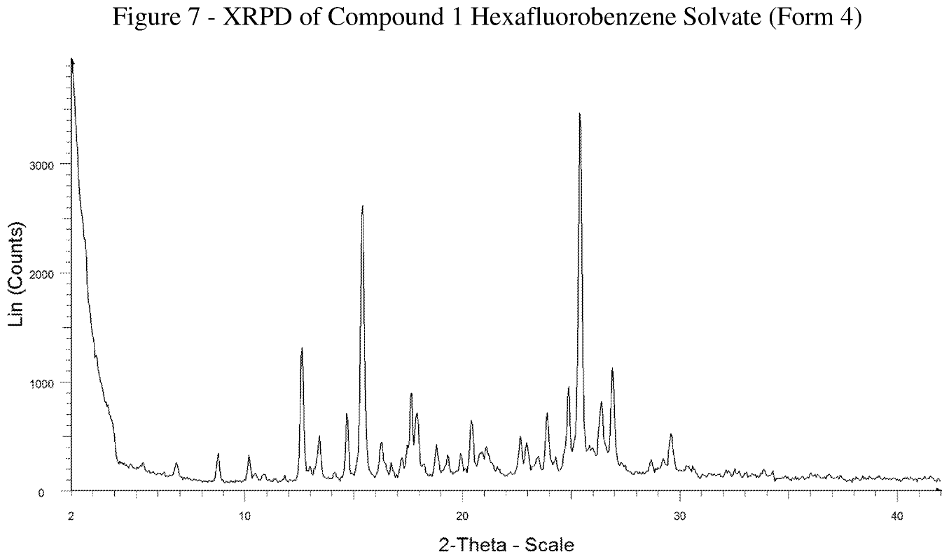

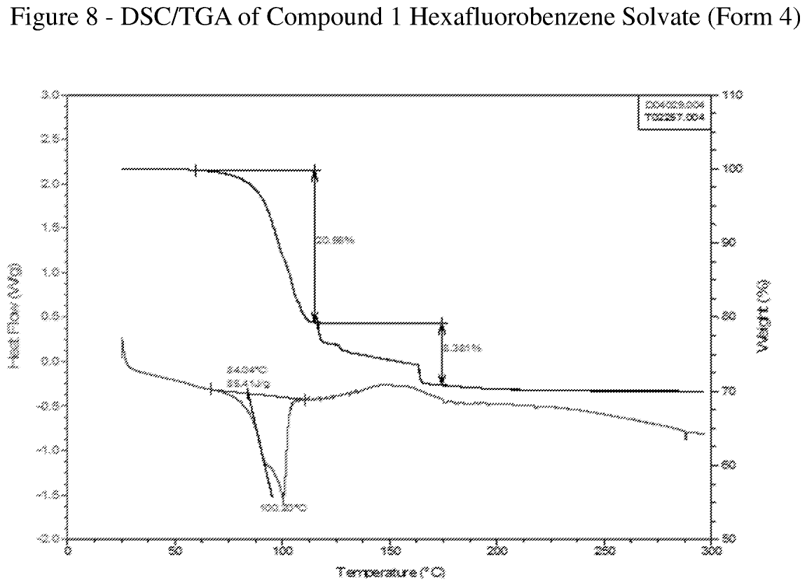

In some embodiments, provided is a Compound 1 hexafluorobenzene solvate. In some embodiments, the hexafluorobenzene solvate of Compound 1 is crystalline Form 4. The crystalline form of the hexafluorobenzene solvate of Compound 1 (Form 4) is characterized as having at least one of the following properties:
(a) an X-ray powder diffraction (XRPD) pattern substantially the same as shown in FIG. 7;
(b) an X-ray powder diffraction (XRPD) pattern with at least two of the characteristic peaks at 12.6±0.1° 2-Theta, 15.4±0.1° 2-Theta, 17.7±0.1° 2-Theta, 24.9±0.1° 2-Theta, 25.4±0.1° 2-Theta, and 26.9±0.1° 2-Theta;
(c) a DSC thermogram substantially the same as the one set forth in FIG. 8;
(d) a DSC thermogram with an endotherm having an onset at about 84° C. and a peak at about 100° C.;
(e) thermo-gravimetric analysis (TGA) thermogram substantially the same as the one set forth in FIG. 8;
   or
(f) combinations thereof.

In some embodiments, the crystalline form of the hexafluorobenzene solvate of Compound 1 (Form 4) is characterized as having at least two of the properties selected from (a) to (e). In some embodiments, the crystalline form of the hexafluorobenzene solvate of Compound 1 (Form 4) is characterized as having at least three of the properties selected from (a) to (e). In some embodiments, the crystalline form of the hexafluorobenzene solvate of Compound 1 (Form 4) is characterized as having at least four of the properties selected from (a) to (e). In some embodiments, the crystalline form of the hexafluorobenzene solvate of Compound 1 (Form 4) is characterized as having properties (a) to (e).

In some embodiments, the crystalline form of the hexafluorobenzene solvate of Compound 1 (Form 4) has an X-Ray powder diffraction (XRPD) pattern substantially the same as shown in FIG. 7. In some embodiments, the crystalline form of the hexafluorobenzene solvate of Compound 1 (Form 4) has an X-ray powder diffraction (XRPD) pattern with characteristic peaks at 12.6±0.1° 2-Theta, 15.4±0.1° 2-Theta, 17.7±0.1° 2-Theta, 24.9±0.1° 2-Theta, 25.4±0.1° 2-Theta, and 26.9±0.1° 2-Theta.

In some embodiments, the crystalline form of the hexafluorobenzene solvate of Compound 1 (Form 4) has a DSC thermogram substantially the same as the one set forth in FIG. 8. In some embodiments, the crystalline form of the hexafluorobenzene solvate of Compound 1 (Form 4) has a DSC thermogram with an endotherm having an onset at about 84° C. and a peak at about 100° C. In some embodiments, the crystalline form of the hexafluorobenzene solvate of Compound 1 (Form 4) has a thermo-gravimetric analysis (TGA) thermogram substantially the same as the one set forth in FIG. 8.

### Acetophenone Solvate of Compound 1, Crystalline Form 5

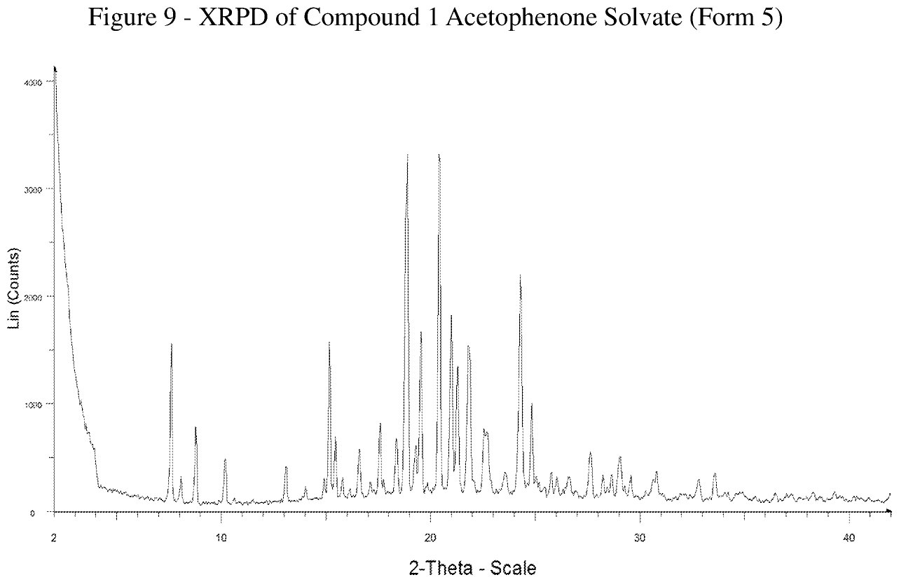

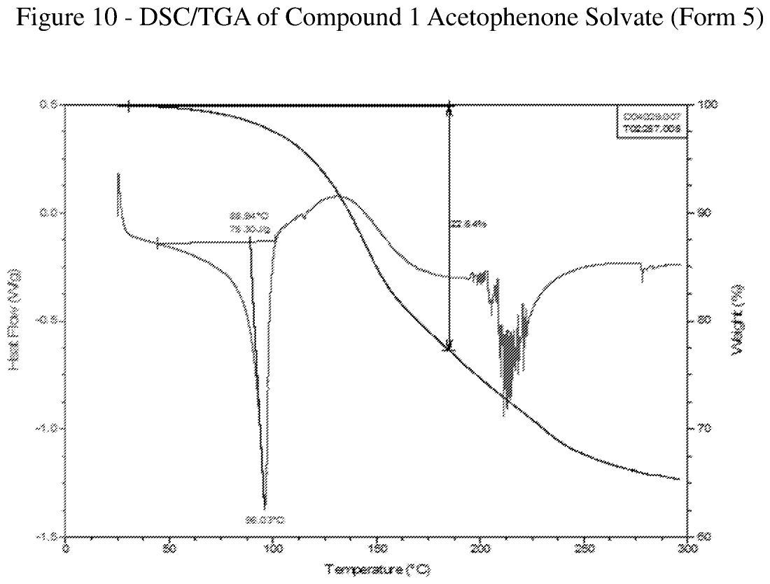

In some embodiments, provided is a Compound 1 acetophenone solvate. In some embodiments, the acetophenone solvate of Compound 1 is crystalline Form 5. In some embodiments, the crystalline form of the acetophenone solvate of Compound 1 (Form 5) is characterized as having at least one of the following properties:
(a) an X-ray powder diffraction (XRPD) pattern substantially the same as shown in FIG. 9;
(b) an X-ray powder diffraction (XRPD) pattern with at least two of the characteristic peaks at 7.6±0.1° 2-Theta, 8.8±0.1° 2-Theta, 15.2±0.1° 2-Theta, 17.6±0.1° 2-Theta, 18.9±0.1 2-Theta, 19.5±0.1° 2-Theta, 20.4±0.1° 2-Theta, 21.0±0.1° 2-Theta, 21.3±0.1° 2-Theta, 21.8±0.1° 2-Theta, 24.3±0.1° 2-Theta, and 24.8±0.1° 2-Theta;
(c) a DSC thermogram substantially the same as the one set forth in FIG. 10;
(d) a DSC thermogram with an endotherm having an onset at about 89° C. and a peak at about 96° C.;
(e) thermo-gravimetric analysis (TGA) thermogram substantially the same as the one set forth in FIG. 10;
(f) unit cell parameters approximately equal to the following at a temperature of approximately 100(2) K:

| Molecular formula | C₃₃H₃₂N₆O₃ | | | | |
|---|---|---|---|---|---|
| Molecular weight | 560.64 | | | | |
| Crystal system | Triclinic | | | | |
| Space group | P1 | a | 11.3552(5) Å | α | 79.657(3)° |
| | | b | 11.7741(4) Å | β | 70.352(4)° |
| | | c | 12.2064(4) Å | γ | 67.080(4)° |

| | |
|---|---|
| V | 1413.38(11) Å³ |
| Z | 2 |
| Density (calculated) | 1.317 Mg/m³ |
| Absorption coefficient | 0.699 mm⁻¹ |
| Wavelength | 1.54178 Å |
| F(000) | 592 |
| T | 100(2) K |

or
(g) combinations thereof.

In some embodiments, the crystalline form of the acetophenone solvate of Compound 1 (Form 5) is characterized as having at least two of the properties selected from (a) to (f). In some embodiments, the crystalline form of the acetophenone solvate of Compound 1 (Form 5) is characterized as having at least three of the properties selected from (a) to (f). In some embodiments, the crystalline form of the acetophenone solvate of Compound 1 (Form 5) is characterized as having at least four of the properties selected from (a) to (f). In some embodiments, the crystalline form of the acetophenone solvate of Compound 1 (Form 5) is characterized as having at least five of the properties selected from (a) to (f). In some embodiments, the crystalline form of the acetophenone solvate of Compound 1 (Form 5) is characterized as having properties (a) to (f).

In some embodiments, the crystalline form of the acetophenone solvate of Compound 1 (Form 5) has an X-Ray powder diffraction (XRPD) pattern substantially the same as shown in FIG. 9. In some embodiments, the crystalline form of the acetophenone solvate of Compound 1 (Form 5) has an X-ray powder diffraction (XRPD) pattern with characteristic peaks at 7.6±0.1° 2-Theta, 8.8±0.1° 2-Theta, 15.2±0.1° 2-Theta, 17.6±0.1° 2-Theta, 18.9±0.1° 2-Theta, 19.5±0.1° 2-Theta, 20.4±0.1° 2-Theta, 21.0±0.1° 2-Theta, 21.3±0.1° 2-Theta, 21.8±0.1° 2-Theta, 24.3±0.1° 2-Theta, and 24.8±0.1° 2-Theta.

In some embodiments, the crystalline form of the acetophenone solvate of Compound 1 (Form 5) has a DSC thermogram substantially the same as the one set forth in FIG. 10. In some embodiments, the crystalline form of the acetophenone solvate of Compound 1 (Form 5) has a DSC thermogram with an endotherm having an onset at about 89° C. and a peak at about 96° C. In some embodiments, endotherm event of the crystalline form of the acetophenone solvate of Compound 1 (Form 5) is between about 50-110° C. In some embodiments, the crystalline form of the acetophenone solvate of Compound 1 (Form 5) has a thermo-gravimetric analysis (TGA) thermogram substantially the same as the one set forth in FIG. 10. In some embodiments, the crystalline form of the acetophenone solvate of Compound 1 (Form 5) has unit cell parameters approximately equal to the following at a temperature of approximately 100(2) K:

| Molecular formula | C₃₃H₃₂N₆O₃ | | | | |
|---|---|---|---|---|---|
| Molecular weight | 560.64 | | | | |
| Crystal system | Triclinic | | | | |
| Space group | P1 | a | 11.3552(5) Å | α | 79.657(3)° |
| | | b | 11.7741(4) Å | β | 70.352(4)° |
| | | c | 12.2064(4) Å | γ | 67.080(4)° |

| | |
|---|---|
| V | 1413.38(11) Å³ |
| Z | 2 |
| Density (calculated) | 1.317 Mg/m³ |
| Absorption coefficient | 0.699 mm⁻¹ |
| Wavelength | 1.54178 Å |
| F(000) | 592 |
| T | 100(2) K |

### Chlorobenzene Solvate of Compound 1, Crystalline Form 6

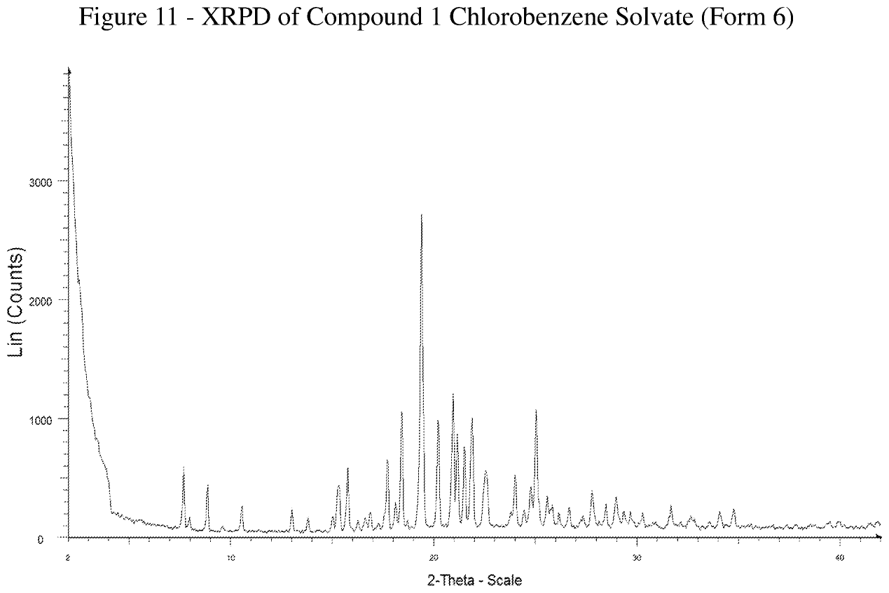

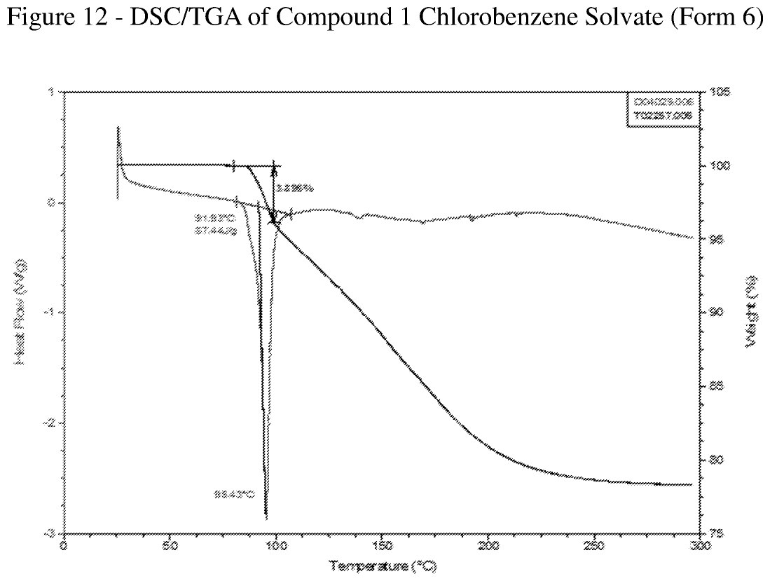

In some embodiments, provided is a Compound 1 chlorobenzene solvate. In some embodiments, the chlorobenzene solvate of Compound 1 is crystalline Form 6. In some embodiments, the crystalline form of the chlorobenzene solvate of Compound 1 (Form 6) is characterized as having at least one of the following properties:
(a) X-ray powder diffraction (XRPD) pattern substantially the same as shown in FIG. 11;
(b) an X-ray powder diffraction (XRPD) pattern with at least two of the characteristic peaks at 18.4±0.1° 2-Theta, 19.4±0.1° 2-Theta, 20.2±0.1° 2-Theta, 20.9±0.1° 2-Theta, 21.2±0.1° 2-Theta, 21.9±0.1° 2-Theta, and 25.0±0.1° 2-Theta;
(c) substantially the same X-ray powder diffraction (XRPD) pattern post storage at 40° C. and 75% RH for 7 days;
(d) a DSC thermogram substantially the same as the one set forth in FIG. 12;
(e) a DSC thermogram with an endotherm having an onset at about 92° C. and a peak at about 95° C.;
(f) thermo-gravimetric analysis (TGA) thermogram substantially the same as the one set forth in FIG. 12;
   or
(g) combinations thereof.

In some embodiments, the crystalline form of the chlorobenzene solvate of Compound 1 (Form 6) is characterized as having at least two of the properties selected from (a) to (f). In some embodiments, the crystalline form of the chlorobenzene solvate of Compound 1 (Form 6) is characterized as having at least three of the properties selected from (a) to (f). In some embodiments, the crystalline form of the chlorobenzene solvate of Compound 1 (Form 6) is characterized as having at least four of the properties selected from (a) to (f). In some embodiments, the crystalline form of the chlorobenzene solvate of Compound 1 (Form 6) is characterized as having at least five of the properties selected from (a) to (f). In some embodiments, the crystalline form of the chlorobenzene solvate of Compound 1 (Form 6) is characterized as having properties (a) to (f).

In some embodiments, the crystalline form of the chlorobenzene solvate of Compound 1 (Form 6) has an X-ray powder diffraction (XRPD) pattern substantially the same as shown in FIG. 11. In some embodiments, the crystalline form of the chlorobenzene solvate of Compound 1 (Form 6) has an X-ray powder diffraction (XRPD) pattern with characteristic peaks at 18.4±0.1° 2-Theta, 19.4±0.1° 2-Theta, 20.2±0.1° 2-Theta, 20.9±0.1° 2-Theta, 21.2±0.1° 2-Theta, 21.9±0.1° 2-Theta, and 25.0±0.1° 2-Theta.

In some embodiments, the crystalline form of the chlorobenzene solvate of Compound 1 (Form 6) has a DSC thermogram substantially the same as the one set forth in FIG. 12. In some embodiments, the crystalline form of the chlorobenzene solvate of Compound 1 (Form 6) has a DSC thermogram with an endotherm having an onset at about 92° C. and a peak at about 95° C. In some embodiments, the crystalline form of the chlorobenzene solvate of Compound 1 (Form 6) has a thermo-gravimetric analysis (TGA) thermogram substantially the same as the one set forth in FIG. 12.

### Acetophenone Solvate of Compound 1, Crystalline Form 7

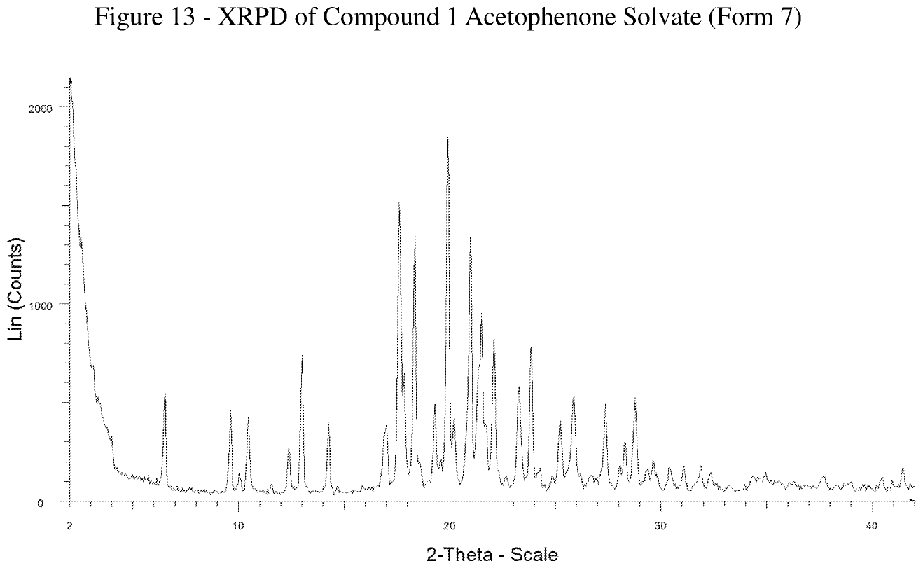

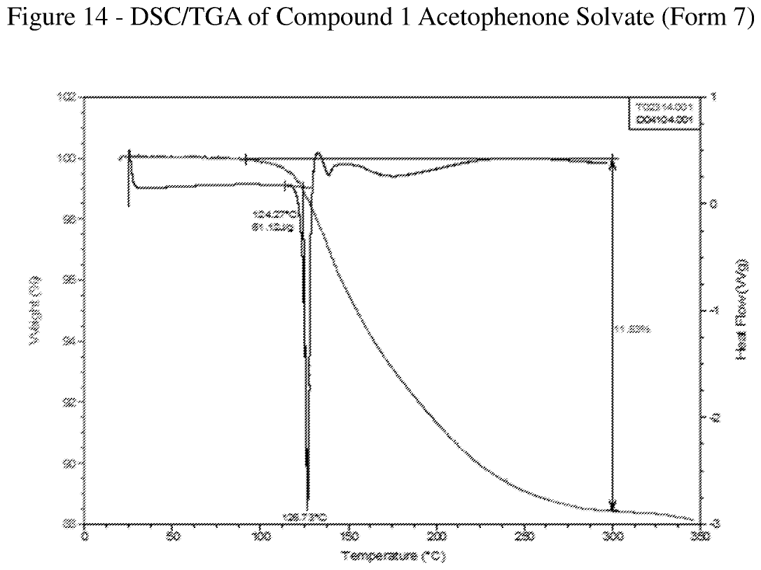

In some embodiments, provided is a Compound 1 acetophenone solvate. In some embodiments, the acetophenone solvate of Compound 1 is crystalline Form 7. In some embodiments, the crystalline form of the acetophenone solvate of Compound 1 (Form 7) is characterized as having at least one of the following properties:
(a) an X-ray powder diffraction (XRPD) pattern substantially the same as shown in FIG. 13;
(b) an X-ray powder diffraction (XRPD) pattern with at least two of the characteristic peaks at 6.5±0.1° 2-Theta, 13.0±0.1° 2-Theta, 17.6±0.1° 2-Theta, 17.9±0.1° 2-Theta, 18.4±0.1° 2-Theta, 19.9±0.1° 2-Theta, 21.0±0.1° 2-Theta, 21.5±0.1° 2-Theta, 22.1±0.1° 2-Theta, 23.3±0.1° 2-Theta and 23.9±0.1° 2-Theta;
(c) a DSC thermogram substantially the same as the one set forth in FIG. 14;
(d) a DSC thermogram with an endotherm having an onset at about 124° C. and a peak at about 127° C.;
(e) thermo-gravimetric analysis (TGA) thermogram substantially the same as the one set forth in FIG. 14; or
(f) combinations thereof.

In some embodiments, the crystalline form of the acetophenone solvate of Compound 1 (Form 7) is characterized as having at least two of the properties selected from (a) to (e). In some embodiments, the crystalline form of the acetophenone solvate of Compound 1 (Form 7) is characterized as having at least three of the properties selected from (a) to (e). In some embodiments, the crystalline form of the acetophenone solvate of Compound 1 (Form 7) is characterized as having at least four of the properties selected from (a) to (e). In some embodiments, the crystalline form of the acetophenone solvate of Compound 1 (Form 7) is characterized as having properties (a) to (e).

In some embodiments, the crystalline form of the acetophenone solvate of Compound 1 (Form 7) has an X-ray powder diffraction (XRPD) pattern substantially the same as shown in FIG. 13. In some embodiments, the crystalline form of the acetophenone solvate of Compound 1 (Form 7) has an X-ray powder diffraction (XRPD) pattern with characteristic peaks at 6.5±0.1° 2-Theta, 13.0±0.1° 2-Theta, 17.6±0.1° 2-Theta, 17.9±0.1° 2-Theta, 18.4±0.1 2-Theta, 19.9±0.1° 2-Theta, 21.0±0.1° 2-Theta, 21.5±0.1° 2-Theta, 22.1±0.1° 2-Theta, 23.3±0.1° 2-Theta and 23.9±0.1° 2-Theta.

In some embodiments, the crystalline form of the acetophenone solvate of Compound 1 (Form 7) has a DSC thermogram substantially the same as the one set forth in FIG. 14. In some embodiments, the crystalline form of the acetophenone solvate of Compound 1 (Form 7) has a DSC thermogram with an endotherm having an onset at about 124° C. and a peak at about 127° C. In some embodiments, the crystalline form of the acetophenone solvate of Compound 1 (Form 7) has a thermo-gravimetric analysis (TGA) thermogram substantially the same as the one set forth in FIG. 14.

### Dimethylacetamide Solvate of Compound 1, Crystalline Form 8

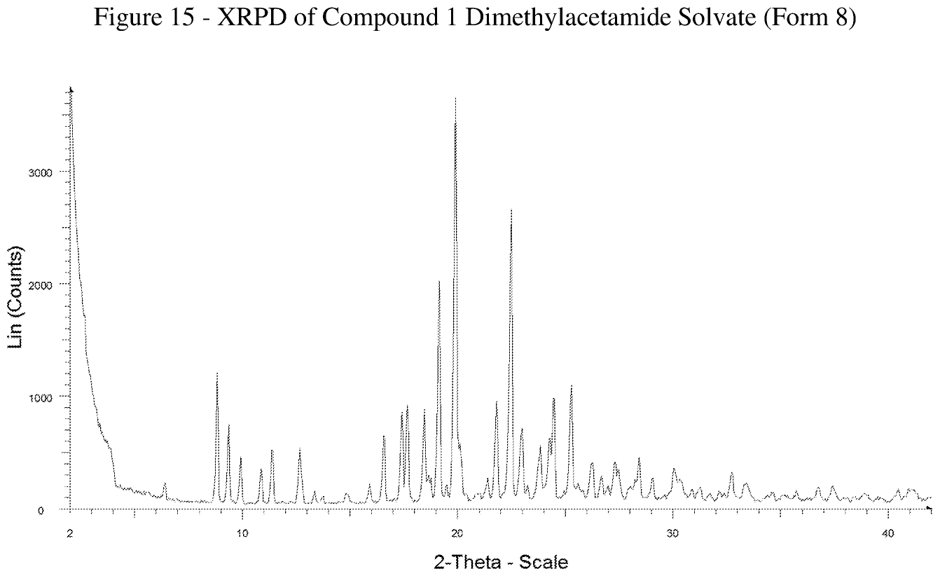

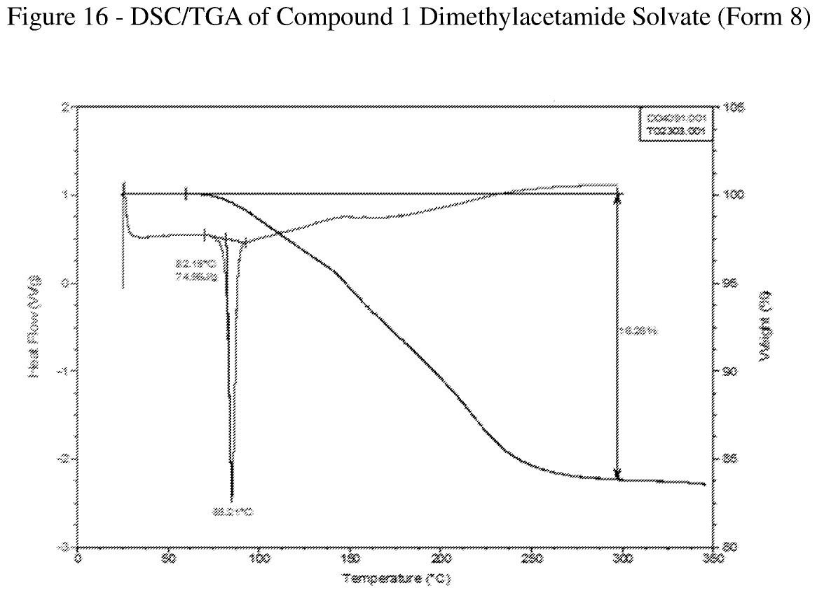

In some embodiments, provided is a Compound 1 dimethylacetamide solvate. In some embodiments, the dimethylacetamide solvate of Compound 1 is crystalline Form 8. In some embodiments, the crystalline form of the dimethylacetamide solvate of Compound 1 (Form 8) is characterized as having at least one of the following properties:
(a) X-ray powder diffraction (XRPD) pattern substantially the same as shown in FIG. 15;
(b) an X-ray powder diffraction (XRPD) pattern with at least two of the characteristic peaks at 8.8±0.1° 2-Theta, 19.2±0.1° 2-Theta, 19.9±0.1° 2-Theta, 22.5±0.1° 2-Theta, 24.5±0.1° 2-Theta, and 25.3±0.1° 2-Theta;
(c) a DSC thermogram substantially the same as the one set forth in FIG. 16;
(d) a DSC thermogram with an endotherm having an onset at about 82° C. and a peak at about 85° C.;
(e) a thermo-gravimetric analysis (TGA) thermogram substantially the same as the one set forth in FIG. 16;
(f) unit cell parameters approximately equal to the following at a temperature of approximately 100(2) K:

| Molecular formula | C₂₉H₃₃N₇O₃ | | | | |
|---|---|---|---|---|---|
| Molecular weight | 527.62 | | | | |
| Crystal system | Triclinic | | | | |
| Space group | P1 | a | 9.3627(3) Å | α | 70.831(3)° |
| | | b | 10.9543(4) Å | β | 76.034(3)° |
| | | c | 14.7742(5) Å | γ | 70.721(3)° |

| | |
|---|---|
| V | 1335.88(9) Å³ |
| Z | 2 |
| Density (calculated) | 1.312 Mg/m³ |
| Absorption coefficient | 0.711 mm⁻¹ |
| Wavelength | 1.54178 Å |
| F(000) | 560 |
| T | 100(2) K |

or
(g) combinations thereof.

In some embodiments, the crystalline form of the dimethylacetamide solvate of Compound 1 (Form 8) is characterized as having at least two of the properties selected from (a) to (f). In some embodiments, the crystalline form of the dimethylacetamide solvate of Compound 1 (Form 8) is characterized as having at least three of the properties selected from (a) to (f). In some embodiments, the crystalline form of the dimethylacetamide solvate of Compound 1 (Form 8) is characterized as having at least four of the properties selected from (a) to (f). In some embodiments, the crystalline form of the dimethylacetamide solvate of Compound 1 (Form 8) is characterized as having at least five of the properties selected from (a) to (f). In some embodiments, the crystalline form of the dimethylacetamide solvate of Compound 1 (Form 8) is characterized as having properties (a) to (f).

In some embodiments, the crystalline form of the dimethylacetamide solvate of Compound 1 (Form 8) has an X-ray powder diffraction (XRPD) pattern substantially the same as shown in FIG. 15. In some embodiments, the crystalline form of the dimethylacetamide solvate of Compound 1 (Form 8) has an X-ray powder diffraction (XRPD) pattern with characteristic peaks at 8.8±0.1° 2-Theta, 19.2±0.1° 2-Theta, 19.9±0.1° 2-Theta, 22.5±0.10 2-Theta, 24.5±0.1° 2-Theta, and 25.3±0.1° 2-Theta.

In some embodiments, the crystalline form of the dimethylacetamide solvate of Compound 1 (Form 8) has a DSC thermogram substantially the same as the one set forth in FIG. 16. In some embodiments, the crystalline form of the dimethylacetamide solvate of Compound 1 (Form 8) has a DSC thermogram with an endotherm having an onset at about 82° C. and a peak at about 85° C. In some embodiments, the crystalline form of the dimethylacetamide solvate of Compound 1 (Form 8) has a thermo-gravimetric analysis (TGA) thermogram substantially the same as the one set forth in FIG. 16. In some embodiments, the crystalline form of the dimethylacetamide solvate of Compound 1 (Form 8) has unit cell parameters approximately equal to the following at a temperature of approximately 100(2) K:

| Molecular formula | C₂₉H₃₃N₇O₃ | | | | |
|---|---|---|---|---|---|
| Molecular weight | 527.62 | | | | |
| Crystal system | Triclinic | | | | |
| Space group | P1 | a | 9.3627(3) Å | α | 70.831(3)° |
| | | b | 10.9543(4) Å | β | 76.034(3)° |
| | | c | 14.7742(5) Å | γ | 70.721(3)° |

| | |
|---|---|
| V | 1335.88(9) Å³ |
| Z | 2 |
| Density (calculated) | 1.312 Mg/m³ |
| Absorption coefficient | 0.711 mm⁻¹ |
| Wavelength | 1.54178 Å |
| F(000) | 560 |
| T | 100(2) K |

### Benzyl Acetate Solvate of Compound 1, Crystalline Form 9

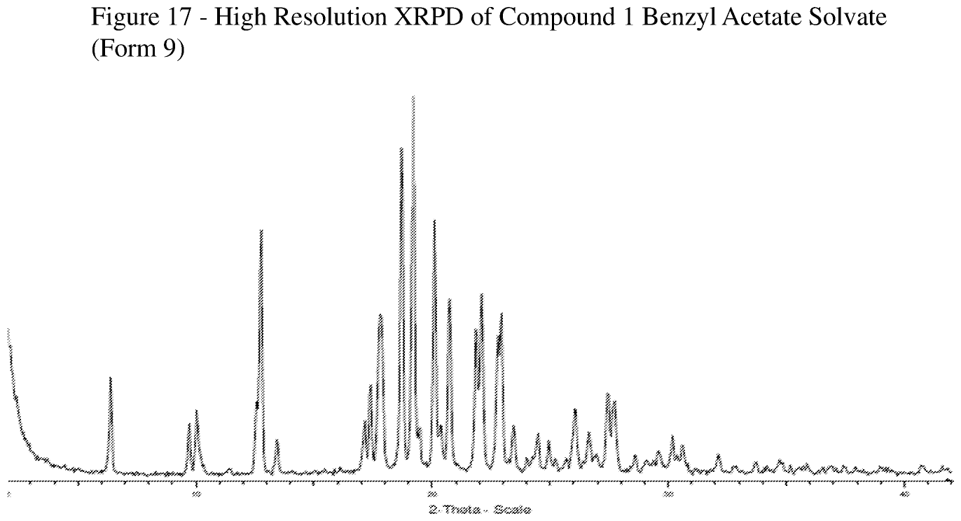

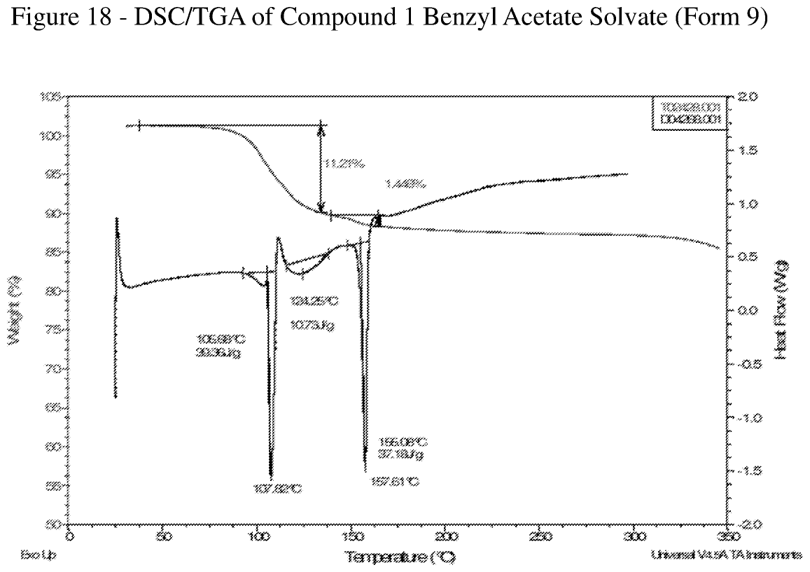

In some embodiments, provided is a Compound 1 benzyl acetate solvate. In some embodiments, the benzyl acetate solvate of Compound 1 is crystalline Form 9. In some embodiments, the crystalline form of the benzyl acetate solvate of Compound 1 (Form 9) is characterized as having at least one of the following properties:
(a) X-ray powder diffraction (XRPD) pattern substantially the same as shown in FIG. 17;
(b) an X-ray powder diffraction (XRPD) pattern with at least two of the characteristic peaks at 12.8±0.1° 2-Theta, 17.8±0.1° 2-Theta, 18.7±0.1° 2-Theta, 19.2±0.1° 2-Theta, 20.1±0.1° 2-Theta, 20.7±0.1° 2-Theta, 22.1±0.1° 2-Theta and 22.9±0.1° 2-Theta;
(c) a DSC thermogram substantially the same as the one set forth in FIG. 18;
(d) a DSC thermogram with an endotherm having an onset at about 106° C. and a peak at about 108° C. and an endotherm having an onset at about 155° C. and a peak at about 158° C.;
(e) a thermo-gravimetric analysis (TGA) thermogram substantially the same as the one set forth in FIG. 18;
   or
(f) combinations thereof.

In some embodiments, the crystalline form of the benzyl acetate solvate of Compound 1 (Form 9) is characterized as having at least two of the properties selected from (a) to (e). In some embodiments, the crystalline form of the benzyl acetate solvate of Compound 1 (Form 9) is characterized as having at least three of the properties selected from (a) to (e). In some embodiments, the crystalline form of the benzyl acetate solvate of Compound 1 (Form 9) is characterized as having at least four of the properties selected from (a) to (e). In some embodiments, the crystalline form of the benzyl acetate solvate of Compound 1 (Form 9) is characterized as having at least five of the properties selected from (a) to (e). In some embodiments, the crystalline form of the benzyl acetate solvate of Compound 1 (Form 9) is characterized as having properties (a) to (e).

In some embodiments, the crystalline form of the benzyl acetate solvate of Compound 1 (Form 9) has an X-ray powder diffraction (XRPD) pattern substantially the same as shown in FIG. 17. In some embodiments, the crystalline form of the benzyl acetate solvate of Compound 1 (Form 9) has an X-ray powder diffraction (XRPD) pattern with characteristic peaks at 12.8±0.1° 2-Theta, 17.8±0.1° 2-Theta, 18.7±0.1° 2-Theta, 19.2±0.1° 2-Theta, 20.1±0.1° 2-Theta, 20.7±0.1° 2-Theta, 22.1±0.1° 2-Theta and 22.9±0.1° 2-Theta.

In some embodiments, the crystalline form of the benzyl acetate solvate of Compound 1 (Form 9) has a DSC thermogram substantially the same as the one set forth in FIG. 18. In some embodiments, the crystalline form of the benzyl acetate solvate of Compound 1 (Form 9) has a DSC thermogram with an endotherm having an onset at about 106° C. and a peak at about 108° C. and an endotherm having an onset at about 155° C. and a peak at about 158° C. In some embodiments, the crystalline form of the benzyl acetate solvate of Compound 1 (Form 9) has a thermo-gravimetric analysis (TGA) thermogram substantially the same as the one set forth in FIG. 18. In some embodiments, the crystalline form of the benzyl acetate solvate of Compound 1 does not change after storage at 40° C. and 75% RH. In some embodiments, the benzyl acetate solvate of Compound 1 (Form 9) is a hemi-solvate.

### 1,1,2-Trichloroethane Solvate of Compound 1, Crystalline Form 10

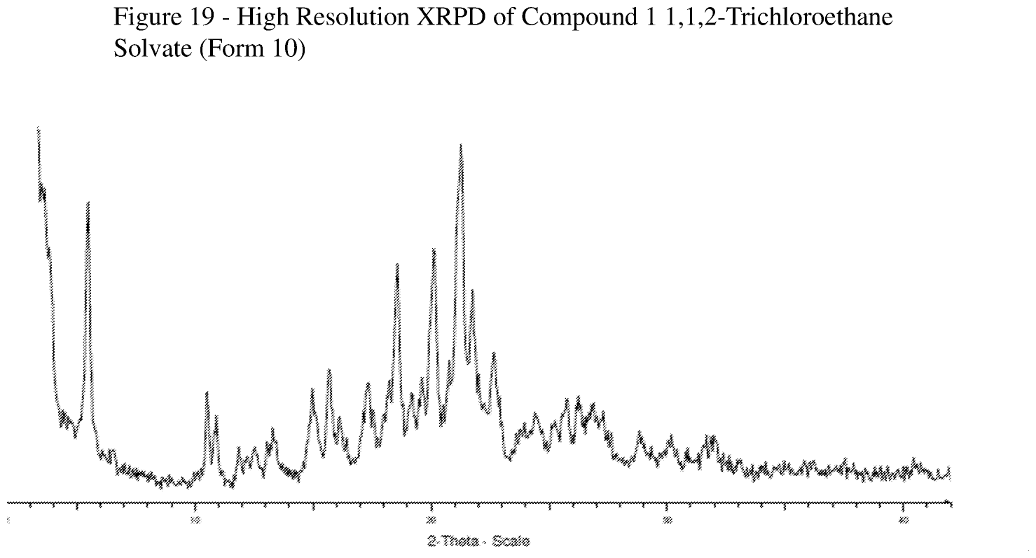

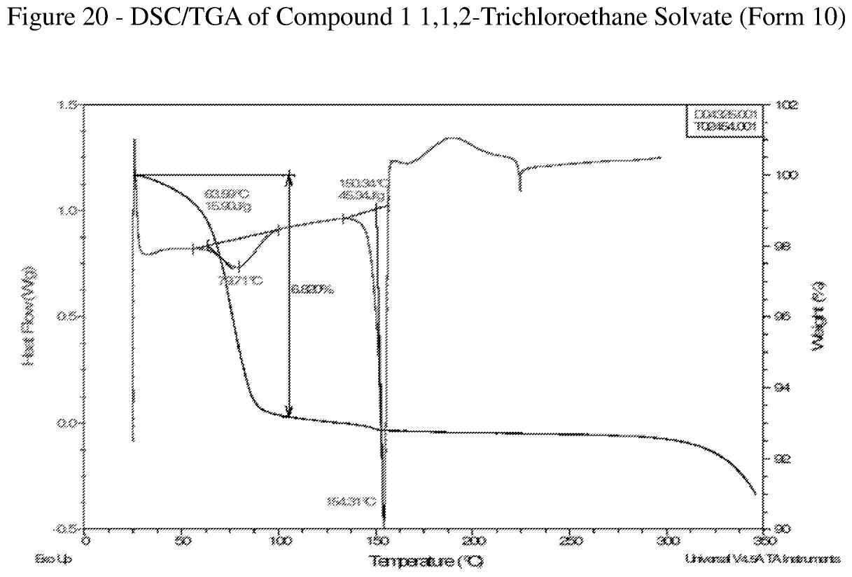

In some embodiments, provided is a Compound 1 1,1,2-trichloroethane solvate. In some embodiments, the 1,1,2-trichloroethane solvate of Compound 1 is crystalline Form 10. In some embodiments, the crystalline form of the 1,1,2-trichloroethane solvate of Compound 1 (Form 10) is characterized as having at least one of the following properties:
(a) X-ray powder diffraction (XRPD) pattern substantially the same as shown in FIG. 19;
(b) an X-ray powder diffraction (XRPD) pattern with at least two of the characteristic peaks at 5.4±0.1° 2-Theta, 18.6±0.1° 2-Theta, 20.1±0.1° 2-Theta, 20.8±0.1° 2-Theta, 21.3±0.1° 2-Theta, 21.7±0.1° 2-Theta, and 22.6±0.1° 2-Theta;
(c) a DSC thermogram substantially the same as the one set forth in FIG. 20;
(d) a DSC thermogram with an endotherm having an onset at about 64° C. and a peak at about 80° C., and an endotherm having an onset at about 150° C. and a peak at about 154° C.;
(e) a thermo-gravimetric analysis (TGA) thermogram substantially the same as the one set forth in FIG. 20;
   or
(f) combinations thereof.

In some embodiments, the crystalline form of the 1,1,2-trichloroethane solvate of Compound 1 (Form 10) is characterized as having at least two of the properties selected from (a) to (e). In some embodiments, the crystalline form of the 1,1,2-trichloroethane solvate of Compound 1 (Form 10) is characterized as having at least three of the properties selected from (a) to (e). In some embodiments, the crystalline form of the 1,1,2-trichloroethane solvate of Compound 1 (Form 10) is characterized as having at least four of the properties selected from (a) to (e). In some embodiments, the crystalline form of the 1,1,2-trichloroethane solvate of Compound 1 (Form 10) is characterized as having at least five of the properties selected from (a) to (e). In some embodiments, the crystalline form of the 1,1,2-trichloroethane solvate of Compound 1 (Form 10) is characterized as having properties (a) to (e).

In some embodiments, the crystalline form of the 1,1,2-trichloroethane solvate of Compound 1 (Form 10) has an X-ray powder diffraction (XRPD) pattern substantially the same as shown in FIG. 19. In some embodiments, the crystalline form of the 1,1,2-trichloroethane solvate of Compound 1 (Form 10) has an X-ray powder diffraction (XRPD) pattern with characteristic peaks at 5.4±0.1° 2-Theta, 18.6±0.1° 2-Theta, 20.1±0.1° 2-Theta, 20.8±0.1° 2-Theta, 21.3±0.1° 2-Theta, 21.7±0.1° 2-Theta, and 22.6±0.1° 2-Theta.

In some embodiments, the crystalline form of the 1,1,2-trichloroethane solvate of Compound 1 (Form 10) has a DSC thermogram substantially the same as the one set forth in FIG. 20. In some embodiments, the crystalline form of the 1,1,2-trichloroethane solvate of Compound 1 (Form 10) has a DSC thermogram with an endotherm having an onset at about 64° C. and a peak at about 80° C., and an endotherm having an onset at about 150° C. and a peak at about 154° C. In some embodiments, the crystalline form of the 1,1,2-trichloroethane solvate of Compound 1 (Form 10) has a thermo-gravimetric analysis (TGA) thermogram substantially the same as the one set forth in FIG. 20. In some embodiments, the crystalline form of the 1,1,2-trichloroethane solvate of Compound 1 changes to a Form A after storage at 40° C. and 75% RH. In some embodiments, the molar ratio of 1,1,2-trichloroethane and Compound 1 in the crystalline form is about 0.3 to 0.4, e.g., about 0.34.

### Preparation of Crystalline Forms

In some embodiments, solvated crystalline forms of 1-((R)-3-(4-amino-3-(4-phenoxyphenyl)-1H-pyrazolo[3,4-d]pyrimidin-1-yl)piperidin-1-yl)prop-2-en-1-one are prepared as outlined in the Examples. It is noted that solvents, temperatures and other reaction conditions presented herein may vary.

### Suitable Solvents

Therapeutic agents that are administrable to mammals, such as humans, must be prepared by following regulatory guidelines. Such government regulated guidelines are referred to as Good Manufacturing Practice (GMP). GMP guidelines outline acceptable contamination levels of active therapeutic agents, such as, for example, the amount of residual solvent in the final product. Preferred solvents are those that are suitable for use in GMP facilities and consistent with industrial safety concerns. Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005).

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene; carbon tetrachloride; 1,2-dichloroethane; 1,1-dichloroethene; and 1,1,1-trichloroethane.

Examples of Class 2 solvents are: acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidine, nitromethane, pyridine, sulfolane, tetrahydrofuran, tetralin, toluene, 1,1,2-trichloroethene and xylene.

Class 3 solvents, which possess low toxicity, include: acetic acid, acetone, anisole, 1-butanol, 2-butanol, butyl acetate, tert-butylmethyl ether (MTBE), cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, and propyl acetate.

Residual solvents in active pharmaceutical ingredients (APIs) originate from the manufacture of API. In some cases, the solvents are not completely removed by practical manufacturing techniques. Appropriate selection of the solvent for the synthesis of APIs may enhance the yield, or determine characteristics such as crystal form, purity, and solubility. Therefore, the solvent is a critical parameter in the synthetic process.

In some embodiments, compositions comprising Compound 1 comprise an organic solvent(s). In some embodiments, compositions comprising Compound 1 comprise a residual amount of an organic solvent(s). In some embodiments, compositions comprising Compound 1 comprise a residual amount of a Class 2 solvent. In some embodiments, compositions comprising Compound 1 comprise a residual amount of a Class 3 solvent. In some embodiments, the organic solvent is a Class 3 solvent. In some embodiments, the Class 3 solvent is selected from the group consisting of acetic acid, acetone, anisole, 1-butanol, 2-butanol, butyl acetate, tert-butylmethyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran. In some embodiments, the Class 3 solvent is selected from ethyl acetate, isopropyl acetate, tert-butylmethylether, heptane, isopropanol, and ethanol.

Other solvents include acetophenone, benzonitrile, benzyl acetate, benzyl alcohol, t-butanol, butyronitrile, chlorobenzotrifluoride, cyclopentylmethyl ether, cyclohexanone, 1,2-dichlorobenzene, ethylene glycol, glycerol, dimethyl carbonate, hexafluorobenzene, methyl-THF, N-methylpyrrolidone, perfluorohexane, propionitrile, 1,1,2-trichloroethane, trifluoroethanol and trifluorotoluene.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

As used herein, the term “agonist” refers to a compound, the presence of which results in a biological activity of a protein that is the same as the biological activity resulting from the presence of a naturally occurring ligand for the protein, such as, for example, Btk.

As used herein, the term “partial agonist” refers to a compound the presence of which results in a biological activity of a protein that is of the same type as that resulting from the presence of a naturally occurring ligand for the protein, but of a lower magnitude.

As used herein, the term “antagonist” refers to a compound, the presence of which results in a decrease in the magnitude of a biological activity of a protein. In certain embodiments, the presence of an antagonist results in complete inhibition of a biological activity of a protein, such as, for example, Btk. In certain embodiments, an antagonist is an inhibitor.

As used herein, “amelioration” of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

“Bioavailability” refers to the percentage of Compound 1 dosed that is delivered into the general circulation of the animal or human being studied. The total exposure (AUC_{(0-∞)}) of a drug when administered intravenously is usually defined as 100% bioavailable (F %). “Oral bioavailability” refers to the extent to which Compound 1 is absorbed into the general circulation when the pharmaceutical composition is taken orally as compared to intravenous injection.

“Blood plasma concentration” refers to the concentration of Compound 1 in the plasma component of blood of a subject. It is understood that the plasma concentration of Compound 1 may vary significantly between subjects, due to variability with respect to metabolism and/or possible interactions with other therapeutic agents. In accordance with one embodiment disclosed herein, the blood plasma concentration of Compound 1 may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of Compound 1 may vary from subject to subject.

The term “Bruton's tyrosine kinase,” as used herein, refers to Bruton's tyrosine kinase from Homo sapiens, as disclosed in, e.g., U.S. Pat. No. 6,326,469 (GenBank Accession No. NP_000052).

The term “Bruton's tyrosine kinase homolog,” as used herein, refers to orthologs of Bruton's tyrosine kinase, e.g., the orthologs from mouse (GenBank Acession No. AAB47246), dog (GenBank Acession No. XP_549139.), rat (GenBank Acession No. NP_001007799), chicken (GenBank Acession No. NP_989564), or zebra fish (GenBank Acession No. XP_698117), and fusion proteins of any of the foregoing that exhibit kinase activity towards one or more substrates of Bruton's tyrosine kinase (e.g. a peptide substrate having the amino acid sequence “AVLESEEELYSSARQ”).

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of Compound 1, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

The term “homologous cysteine,” as used herein refers to a cysteine residue found with in a sequence position that is homologous to that of cysteine 481 of Bruton's tyrosine kinase, as defined herein. For example, cysteine 482 is the homologous cysteine of the rat ortholog of Bruton's tyrosine kinase; cysteine 479 is the homologous cysteine of the chicken ortholog; and cysteine 481 is the homologous cysteine in the zebra fish ortholog. In another example, the homologous cysteine of TXK, a Tec kinase family member related to Bruton's tyrosine, is Cys 350. Other examples of kinases having homologous cysteines are shown in FIG. 1. See also the sequence alignments of tyrosine kinases (TK) published on the world wide web at kinase.com/human/kinome/phylogeny.html.

The term “substantially the same,” as used herein to define a figure is intended to mean that the figure is considered the same as a reference figure by a skilled artisan in view of deviations acceptable in the art. Such deviations may be caused by factors related to instruments, operation conditions and human factors, etc., known in the art. For example, one skilled in the art can appreciate that the endotherm onset and peak temperatures as measured by differential scanning calorimetry (DSC) may vary significantly from experiment to experiment. In some embodiments, when positions of characteristic peaks of two figures do not vary more than ±5% or ±1%, it is deemed that the two figures are substantially the same. For example, one skilled in the art can readily identify whether two X-ray diffraction patterns or two DSC thermograms are substantially the same. In some embodiments, when characteristic peaks of two X-ray diffraction patterns do not vary more than ±0.2° 2-Theta or ±0.1° 2-Theta, it is deemed that the X-ray diffraction patterns are substantially the same. The term “characteristic peaks” refers to peaks that are distinguishable from the baseline noise. In some embodiments, “characteristic peaks” refers to peaks having an area, height, or intensity that is at least 30%, at least 25% or at least 20% of the peak having the largest area, height, or intensity, respectively. The term “about” or “˜” when used before a numerical value indicates that the value may vary within a reasonable range, such as within ±10%, ±5% or ±1% of the stated value.

The terms “inhibits”, “inhibiting”, or “inhibitor” of a kinase, as used herein, refer to inhibition of enzymatic phosphotransferase activity.

The term “irreversible inhibitor,” as used herein, refers to a compound that, upon contact with a target protein (e.g., a kinase) causes the formation of a new covalent bond with or within the protein, whereby one or more of the target protein's biological activities (e.g., phosphotransferase activity) is diminished or abolished notwithstanding the subsequent presence or absence of the irreversible inhibitor.

The term “irreversible Btk inhibitor,” as used herein, refers to an inhibitor of Btk that can form a covalent bond with an amino acid residue of Btk. In one embodiment, the irreversible inhibitor of Btk can form a covalent bond with a Cys residue of Btk; in particular embodiments, the irreversible inhibitor can form a covalent bond with a Cys 481 residue (or a homolog thereof) of Btk or a cysteine residue in the homologous corresponding position of another tyrosine kinase.

The term “isolated,” as used herein, refers to separating and removing a component of interest from components not of interest. Isolated substances can be in either a dry or semi-dry state, or in solution, including but not limited to an aqueous solution. The isolated component can be in a homogeneous state or the isolated component can be a part of a pharmaceutical composition that comprises additional pharmaceutically acceptable carriers and/or excipients. By way of example only, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Also, by way of example, a gene is isolated when separated from open reading frames which flank the gene and encode a protein other than the gene of interest.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

The term “prophylactically effective amount,” as used herein, refers that amount of a composition applied to a patient which will relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

The term “subject” as used herein, refers to an animal which is the object of treatment, observation or experiment. By way of example only, a subject may be, but is not limited to, a mammal including, but not limited to, a human.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

As used herein, the term “target protein” refers to a molecule or a portion of a protein capable of being bound by a selective binding compound. In certain embodiments, a target protein is Btk.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the IC₅₀ refers to an amount, concentration or dosage of a particular test compound that achieves a 50% inhibition of a maximal response, such as inhibition of Btk, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

### Pharmaceutical Compositions/Formulations

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

A pharmaceutical composition or pharmaceutical formulation, as used herein, refers to a mixture of solvated Compound 1 with other chemical components, such as carriers, stabilizers, diluents, dispersing agents, suspending agents, thickening agents, and/or excipients. The pharmaceutical composition facilitates administration of the compound to a mammal. In practicing the methods of treatment or use provided herein, therapeutically effective amounts of Compound 1 are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures. The solvates of Compound 1 described herein can be administered in the pharmaceutical compositions described in U.S. Pat. No. 7,514,444.

The term “pharmaceutical combination” as used herein, means a product that results from the mixing or combining of more than one active ingredient and includes both fixed and non-fixed combinations of the active ingredients. The term “fixed combination” means that the active ingredients, e.g. solvated Compound 1 and a co-agent, are both administered to a patient simultaneously in the form of a single entity or dosage. The term “non-fixed combination” means that the active ingredients, e.g. solvated Compound 1 and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients. The solvates of Compound 1 described herein can be administered in the pharmaceutical combinations described in U.S. Pat. No. 7,514,444.

In some embodiments, a solvate of Compound 1 is incorporated into pharmaceutical compositions to provide solid oral dosage forms. In other embodiments, a solvate of Compound 1 is used to prepare pharmaceutical compositions other than solid oral dosage forms. The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations. In some embodiments, the pharmaceutical composition comprising a pharmaceutically acceptable carrier and a solvate provided herein is in a solid form or a suspension in a liquid excipient. In some embodiments, the pharmaceutical composition is in a liquid solution form and comprises a pharmaceutically acceptable carrier and is prepared from a solvate provided herein.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

### Dosage Forms

The pharmaceutical compositions described herein can be formulated for administration to a mammal via any conventional means including, but not limited to, oral, parenteral (e.g., intravenous, subcutaneous, or intramuscular), buccal, intranasal, rectal or transdermal administration routes. As used herein, the term “subject” is used to mean an animal, preferably a mammal, including a human or non-human. The terms patient and subject may be used interchangeably.

Moreover, the pharmaceutical compositions described herein, which include a solvate of Compound 1 can be formulated into any suitable dosage form, including but not limited to, solid oral dosage forms, controlled release formulations, fast melt formulations, effervescent formulations, tablets, powders, pills, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations described herein may be administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of a solvate of Compound 1 with one or more pharmaceutical excipients to form a bulk blend composition. When referring to these bulk blend compositions as homogeneous, it is meant that the particles of a solvate of Compound 1 are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a solvate of Compound 1 and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of a solvate of Compound 1. In one embodiment, some or all of the particles of the solvate of Compound 1 are coated. In another embodiment, some or all of the particles of the solvate of Compound 1 are microencapsulated. In still another embodiment, the particles of the solvate of Compound 1 are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release Compound 1 from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like. In some embodiments provided herein, the disintegrating agent is selected from the group consisting of natural starch, a pregelatinized starch, a sodium starch, methylcrystalline cellulose, methylcellulose, croscarmellose, croscarmellose sodium, cross-linked sodium carboxymethylcellulose, cross-linked carboxymethylcellulose, cross-linked croscarmellose, cross-linked starch such as sodium starch glycolate, cross-linked polymer such as crospovidone, cross-linked polyvinylpyrrolidone, sodium alginate, a clay, or a gum. In some embodiments provided herein, the disintegrating agent is croscarmellose sodium.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like. In some embodiments provided herein, the lubricant is selected from the group consisting of stearic acid, calcium hydroxide, talc, corn starch, sodium stearyl fumerate, stearic acid, sodium stearates, magnesium stearate, zinc stearate, and waxes. In some embodiments provided herein, the lubricant is magnesium stearate.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like. In some embodiments provided herein, the diluent is selected from the group consisting of lactose, sucrose, dextrose, dextrates, maltodextrin, mannitol, xylitol, sorbitol, cyclodextrins, calcium phosphate, calcium sulfate, starches, modified starches, microcrystalline cellulose, microcellulose, and talc. In some embodiments provided herein, the diluent is microcrystalline cellulose.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. In some embodiments provided herein, the surfactant is selected from the group consisting of sodium lauryl sulfate, sorbitan monooleate, polyoxyethylene sorbitan monooleate, polysorbates, polaxomers, bile salts, glyceryl monostearate, copolymers of ethylene oxide and propylene oxide. In some embodiments provided herein, the surfactant is sodium lauryl sulfate.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxypropylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating can provide a delayed release of a solvate of Compound 1 from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

A capsule may be prepared, for example, by placing the bulk blend of the formulation of solvated Compound 1 inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of a solvate of Compound 1 and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with a solvate of Compound 1 which sufficiently isolate the solvated Compound 1 from other non-compatible excipients. Materials compatible with a solvate of Compound 1 are those that delay the release of the compounds of solvated Compound 1 in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated solvated Compound 1 may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In one embodiment, the particles of a solvate of Compound 1 are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of solvated Compound 1 are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments, a powder including the formulations with a solvate of Compound 1 may be formulated to include one or more pharmaceutical excipients and flavors. Such a powder may be prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH>7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions; Poly Vinyl Acetate Phthalate (PVAP). PVAP dissolves in pH>5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In other embodiments, the formulations described herein, which include solvates of Compound 1 are delivered using a pulsatile dosage form. A pulsatile dosage form is capable of providing one or more immediate release pulses at predetermined time points after a controlled lag time or at specific sites. Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983, each of which is specifically incorporated by reference.

In some embodiments, pharmaceutical formulations are provided that include particles of a solvate of Compound 1 and at least one dispersing agent or suspending agent for oral administration to a subject. The formulations may be a powder and/or granules for suspension, and upon admixture with water, a substantially uniform suspension is obtained.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

### Dosing and Treatment Regimens

In some embodiments, a solvate of Compound 1 is administered to a mammal in an amount that delivers Compound 1 to the mammal in an amount as described herein. In some embodiments, the amount of Compound 1 is from 300 mg/day up to, and including, 1000 mg/day. In some embodiments, the amount of a solvate of Compound 1 that is administered to a mammal is from 420 mg/day up to, and including, 840 mg/day. In some embodiments, the amount of a solvate of Compound 1 that is administered to a mammal delivers Compound 1 in an amount of about 420 mg/day, about 560 mg/day, or about 840 mg/day. In some embodiments, the amount of Compound 1 is about 420 mg/day. In some embodiments, the amount of Compound 1 is about 560 mg/day. In some embodiments, the AUC₀₋₂₄ of Compound 1 is between about 150 and about 3500 ng*h/mL. In some embodiments, the AUC₀₋₂₄ of Compound 1 is between about 500 and about 1100 ng*h/mL. In some embodiments, a solvate of Compound 1 is administered orally. In some embodiments, a solvate of Compound 1 is administered once per day, twice per day, or three times per day. In some embodiments, a solvate of Compound 1 is administered daily. In some embodiments, a solvate of Compound 1 is administered once daily. In some embodiments, a solvate of Compound 1 is administered every other day. In some embodiments, a solvate of Compound 1 is a maintenance therapy.

A solvate of Compound 1 can be used in the preparation of medicaments for the inhibition of Btk or a homolog thereof, or for the treatment of diseases or conditions that would benefit, at least in part, from inhibition of Btk or a homolog thereof, including a subject diagnosed with a hematological malignancy. In addition, a method for treating any of the diseases or conditions described herein in a subject in need of such treatment, involves administration of pharmaceutical compositions containing a solvate of Compound 1, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

The compositions containing a solvate of Compound 1 can be administered for prophylactic, therapeutic, or maintenance treatment. In some embodiments, compositions containing a solvate of Compound 1 are administered for therapeutic applications (e.g., administered to a subject diagnosed with a hematological malignancy). In some embodiments, compositions containing a solvate of Compound 1 are administered for prophylactic applications (e.g., administered to a subject susceptible to or otherwise at risk of developing a hematological malignancy). In some embodiments, compositions containing a solvate of Compound 1 are administered to a patient who is in remission as a maintenance therapy.

Amounts of solvated Compound 1 will depend on the use (e.g., therapeutic, prophylactic, or maintenance). Amounts of a solvate of Compound 1 will depend on severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial). In some embodiments, the amount of a solvate of Compound 1 provides Compound 1 from 300 mg/day up to, and including, 1000 mg/day. In some embodiments, the amount of Compound 1 is from 420 mg/day up to, and including, 840 mg/day. In some embodiments, the amount of Compound 1 is from 400 mg/day up to, and including, 860 mg/day. In some embodiments, the amount of Compound 1 is about 360 mg/day. In some embodiments, the amount of Compound 1 is about 420 mg/day. In some embodiments, the amount of Compound 1 is about 560 mg/day. In some embodiments, the amount of Compound 1 is about 840 mg/day. In some embodiments, the amount of Compound 1 is from 2 mg/kg/day up to, and including, 13 mg/kg/day. In some embodiments, the amount of Compound 1 is from 2.5 mg/kg/day up to, and including, 8 mg/kg/day. In some embodiments, the amount of Compound 1 is from 2.5 mg/kg/day up to, and including, 6 mg/kg/day. In some embodiments, the amount of Compound 1 is from 2.5 mg/kg/day up to, and including, 4 mg/kg/day. In some embodiments, the amount of Compound 1 is about 2.5 mg/kg/day. In some embodiments, the amount of Compound 1 is about 8 mg/kg/day.

In some embodiments, pharmaceutical compositions described herein include about 140 mg of Compound 1. In some embodiments, a capsule formulation is prepared that includes about 140 mg of Compound 1. In some embodiments, 2, 3, 4, or 5 of the capsule formulations are administered daily. In some embodiments, 3 or 4 of the capsules are administered daily. In some embodiments, 3 of the 140 mg capsules are administered once daily. In some embodiments, 4 of the 140 mg capsules are administered once daily. In some embodiments, the capsules are administered once daily. In other embodiments, the capsules are administered multiple times a day.

In some embodiments, a solvate of Compound 1 is administered daily. In some embodiments, a solvate of Compound 1 is administered every other day.

In some embodiments, a solvate of Compound 1 is administered once per day. In some embodiments, a solvate of Compound 1 is administered twice per day. In some embodiments, a solvate of Compound 1 is administered three times per day. In some embodiments, a solvate of Compound 1 is administered four times per per day.

In some embodiments, a solvate of Compound 1 is administered until disease progression, unacceptable toxicity, or individual choice. In some embodiments, a solvate of Compound 1 is administered daily until disease progression, unacceptable toxicity, or individual choice. In some embodiments, a solvate of Compound 1 is administered every other day until disease progression, unacceptable toxicity, or individual choice.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, the severity of the disease, the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative. In some embodiments, each unit dosage form comprises 140 mg of Compound 1. In some embodiments, an individual is administered 1 unit dosage form per day. In some embodiments, an individual is administered 2 unit dosage forms per day. In some embodiments, an individual is administered 3 unit dosage forms per day. In some embodiments, an individual is administered 4 unit dosage forms per day.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Combination Therapy

In certain instances, it is appropriate to administer a solvate of Compound 1 in combination with another therapeutic agent.

In one embodiment, the compositions and methods described herein are also used in conjunction with other therapeutic reagents that are selected for their particular usefulness against the condition that is being treated. In general, the compositions described herein and, in embodiments where combinational therapy is employed, other agents do not have to be administered in the same pharmaceutical composition, and are, because of different physical and chemical characteristics, administered by different routes. In one embodiment, the initial administration is made according to established protocols, and then, based upon the observed effects, the dosage, modes of administration and times of administration, further modified.

In various embodiments, the compounds are administered concurrently (e.g., simultaneously, essentially simultaneously or within the same treatment protocol) or sequentially, depending upon the nature of the disease, the condition of the patient, and the actual choice of compounds used. In certain embodiments, the determination of the order of administration, and the number of repetitions of administration of each therapeutic agent during a treatment protocol, is based upon evaluation of the disease being treated and the condition of the patient.

For combination therapies described herein, dosages of the co-administered compounds vary depending on the type of co-drug employed, on the specific drug employed, on the disease or condition being treated and so forth.

The individual compounds of such combinations are administered either sequentially or simultaneously in separate or combined pharmaceutical formulations. In one embodiment, the individual compounds will be administered simultaneously in a combined pharmaceutical formulation. Appropriate doses of known therapeutic agents will be appreciated by those skilled in the art.

The combinations referred to herein are conveniently presented for use in the form of a pharmaceutical compositions together with a pharmaceutically acceptable diluent(s) or carrier(s).

Disclosed herein, in certain embodiments, is a method for treating a cancer in an individual in need thereof, comprising: administering to the individual an amount of a solvate of Compound 1. In some embodiments, the method further comprises administering a second cancer treatment regimen.

In some embodiments, administering a Btk inhibitor before a second cancer treatment regimen reduces immune-mediated reactions to the second cancer treatment regimen. In some embodiments, administering a solvate of Compound 1 before ofatumumab reduces immune-mediated reactions to ofatumumab.

In some embodiments, the second cancer treatment regimen comprises a chemotherapeutic agent, a steroid, an immunotherapeutic agent, a targeted therapy, or a combination thereof. In some embodiments, the second cancer treatment regimen comprises a B cell receptor pathway inhibitor. In some embodiments, the B cell receptor pathway inhibitor is a CD79A inhibitor, a CD79B inhibitor, a CD19 inhibitor, a Lyn inhibitor, a Syk inhibitor, a PI3K inhibitor, a Blnk inhibitor, a PLCγ inhibitor, a PKCβ inhibitor, a LYN inhibitor, a JAK inhibitor, a MAPK inhibitor, a MEK inhibitor or a NFκB inhibitor or a combination thereof. In some embodiments, the second cancer treatment regimen comprises an antibody, B cell receptor signaling inhibitor, a PI3K inhibitor, an IAP inhibitor, an mTOR inhibitor, a radioimmunotherapeutic, a DNA damaging agent, a proteosome inhibitor, a Cyp3A4 inhibitor, a histone deacetylase inhibitor, a protein kinase inhibitor, a hedgehog inhibitor, an Hsp90 inhibitor, a telomerase inhibitor, a Jak1/2 inhibitor, a protease inhibitor, a PKC inhibitor, a PARP inhibitor, or a combination thereof.

In some embodiments, the second cancer treatment regimen comprises chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

In some embodiments, the second cancer treatment regimen comprises cyclophosphamide, hydroxydaunorubicin, vincristine, and prednisone, and optionally, rituximab.

In some embodiments, the second cancer treatment regimen comprises bendamustine, and rituximab.

In some embodiments, the second cancer treatment regimen comprises fludarabine, cyclophosphamide, and rituximab.

In some embodiments, the second cancer treatment regimen comprises cyclophosphamide, vincristine, and prednisone, and optionally, rituximab.

In some embodiments, the second cancer treatment regimen comprises etoposide, doxorubicin, vinristine, cyclophosphamide, prednisolone, and optionally, rituximab.

In some embodiments, the second cancer treatment regimen comprises dexamethasone and lenalidomide.

In some embodiments, the second cancer treatment comprises a proteasome inhibitor. In some embodiments, the second treatment comprises bortezomib. In some embodiments, the second cancer treatment comprises an epoxyketone. In some embodiments, the second cancer treatment comprises epoxomicin. In some embodiments, the second cancer treatment comprises a tetrapeptide epoxyketone. In some embodiments, the second cancer treatment comprises carfilzomib. In some embodiments, the second cancer treatment comprises disulfram, epigallocatechin-3-gallate, salinosporamide A, ONX 0912m CEP-18770, MLN9708, or MG132.

In some embodiments, the second cancer treatment comprises a Cyp3A4 inhibitor. In some embodiments, the second cancer treatment comprises indinavir, nelfinavir, ritonavir, clarithromycin, itraconazole, ketoconazole, nefazodone. In some embodiments, the second cancer treatment comprises ketoconazole.

In some embodiments, the second cancer treatment comprises a Janus Kinase (JAK) inhibitor. In some embodiments, the second treatment comprises Lestaurtinib, Tofacitinib, Ruxolitinib, CYT387, Baricitinib or Pacritinib.

In some embodiments, the second cancer treatment comprises a histone deacetylase inhibitor (HDAC inhibitor, HDI). In some embodiments, the second cancer treatment comprises a hydroxamic acid (or hydroxamate), such as trichostatin A, vorinostat (SAHA), belinostat (PXD101), LAQ824, and panobinostat (LBH589), a cyclic tetrapeptide, such as trapoxin B, a depsipeptide, a benzamide, such as entinostat (MS-275), CI994, and mocetinostat (MGCD0103), an electrophilic ketone, or an aliphatic acid compound, such as phenylbutyrate and valproic acid,

Additional cancer treatment regimens include Nitrogen Mustards such as for example, bendamustine, chlorambucil, chlormethine, cyclophosphamide, ifosfamide, melphalan, prednimustine, trofosfamide; Alkyl Sulfonates like busulfan, mannosulfan, treosulfan; Ethylene Imines like carboquone, thiotepa, triaziquone; Nitrosoureas like carmustine, fotemustine, lomustine, nimustine, ranimustine, semustine, streptozocin; Epoxides such as for example, etoglucid; Other Alkylating Agents such as for example dacarbazine, mitobronitol, pipobroman, temozolomide; Folic Acid Analogues such as for example methotrexate, permetrexed, pralatrexate, raltitrexed; Purine Analogs such as for example cladribine, clofarabine, fludarabine, mercaptopurine, nelarabine, tioguanine; Pyrimidine Analogs such as for example azacitidine, capecitabine, carmofur, cytarabine, decitabine, fluorouracil, gemcitabine, tegafur; Vinca Alkaloids such as for example vinblastine, vincristine, vindesine, vinflunine, vinorelbine; Podophyllotoxin Derivatives such as for example etoposide, teniposide; Colchicine derivatives such as for example demecolcine; Taxanes such as for example docetaxel, paclitaxel, paclitaxel poliglumex; Other Plant Alkaloids and Natural Products such as for example trabectedin; Actinomycines such as for example dactinomycin; Antracyclines such as for example aclarubicin, daunorubicin, doxorubicin, epirubicin, idarubicin, mitoxantrone, pirarubicin, valrubicin, zorubincin; Other Cytotoxic Antibiotics such as for example bleomycin, ixabepilone, mitomycin, plicamycin; Platinum Compounds such as for example carboplatin, cisplatin, oxaliplatin, satraplatin; Methylhydrazines such as for example procarbazine; Sensitizers such as for example aminolevulinic acid, efaproxiral, methyl aminolevulinate, porfimer sodium, temoporfin; Protein Kinase Inhibitors such as for example dasatinib, erlotinib, everolimus, gefitinib, imatinib, lapatinib, nilotinib, pazonanib, sorafenib, sunitinib, temsirolimus; Other Antineoplastic Agents such as for example alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofurine, topotecan, tretinoin, vorinostat; Estrogens such as for example diethylstilbenol, ethinylestradiol, fosfestrol, polyestradiol phosphate; Progestogens such as for example gestonorone, medroxyprogesterone, megestrol; Gonadotropin Releasing Hormone Analogs such as for example buserelin, goserelin, leuprorelin, triptorelin; Anti-Estrogens such as for example fulvestrant, tamoxifen, toremifene; Anti-Androgens such as for example bicalutamide, flutamide, nilutamide, Enzyme Inhibitors, aminoglutethimide, anastrozole, exemestane, formestane, letrozole, vorozole; Other Hormone Antagonists such as for example abarelix, degarelix; Immunostimulants such as for example histamine dihydrochloride, mifamurtide, pidotimod, plerixafor, roquinimex, thymopentin; Immunosuppressants such as for example everolimus, gusperimus, leflunomide, mycophenolic acid, sirolimus; Calcineurin Inhibitors such as for example ciclosporin, tacrolimus; Other Immunosuppressants such as for example azathioprine, lenalidomide, methotrexate, thalidomide; and Radiopharmaceuticals such as for example, iobenguane.

Additional cancer treatment regimens include interferons, interleukins, Tumor Necrosis Factors, Growth Factors, or the like.

Additional cancer treatment regimens include Immunostimulants such as for example ancestim, filgrastim, lenograstim, molgramostim, pegfilgrastim, sargramostim; Interferons such as for example interferon alfa natural, interferon alfa-2a, interferon alfa-2b, interferon alfacon-1, interferon alfa-n1, interferon beta natural, interferon beta-1a, interferon beta-1b, interferon gamma, peginterferon alfa-2a, peginterferon alfa-2b; Interleukins such as for example aldesleukin, oprelvekin; Other Immunostimulants such as for example BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin; Immunosuppressants such as for example abatacept, abetimus, alefacept, antilymphocyte immunoglobulin (horse), antithymocyte immunoglobulin (rabbit), eculizumab, efalizumab, everolimus, gusperimus, leflunomide, muromab-CD3, mycophenolic acid, natalizumab, sirolimus; TNF alpha Inhibitors such as for example adalimumab, afelimomab, certolizumab pegol, etanercept, golimumab, infliximab; Interleukin Inhibitors such as for example anakinra, basiliximab, canakinumab, daclizumab, mepolizumab, rilonacept, tocilizumab, ustekinumab; Calcineurin Inhibitors such as for example ciclosporin, tacrolimus; Other Immunosuppressants such as for example azathioprine, lenalidomide, methotrexate, thalidomide.

Additional cancer treatment regimens include Adalimumab, Alemtuzumab, Basiliximab, Bevacizumab, Cetuximab, Certolizumab pegol, Daclizumab, Eculizumab, Efalizumab, Gemtuzumab, Ibritumomab tiuxetan, Infliximab, Muromonab-CD3, Natalizumab, Panitumumab, Ranibizumab, Rituximab, Tositumomab, Trastuzumab, or the like, or a combination thereof.

Additional cancer treatment regimens include Monoclonal Antibodies such as for example alemtuzumab, bevacizumab, catumaxomab, cetuximab, edrecolomab, gemtuzumab, ofatumumab, panitumumab, rituximab, trastuzumab, Immunosuppressants, eculizumab, efalizumab, muromab-CD3, natalizumab; TNF alpha Inhibitors such as for example adalimumab, afelimomab, certolizumab pegol, golimumab, infliximab, Interleukin Inhibitors, basiliximab, canakinumab, daclizumab, mepolizumab, tocilizumab, ustekinumab, Radiopharmaceuticals, ibritumomab tiuxetan, tositumomab; Others Monoclonal Antibodies such as for example abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, ofatumumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, zalutumumab.

Additional cancer treatment regimens include agents that affect the tumor micro-enviroment such as cellular signaling network (e.g. phosphatidylinositol 3-kinase (PI3K) signaling pathway, signaling from the B-cell receptor and the IgE receptor). In some embodiments, the second agent is a PI3K signaling inhibitor or a syc kinase inhibitor. In one embodiment, the syk inhibitor is R788. In another embodiment is a PKCy inhibitor such as by way of example only, enzastaurin.

Examples of agents that affect the tumor micro-environment include PI3K signaling inhibitor, syc kinase inhibitor, Protein Kinase Inhibitors such as for example dasatinib, erlotinib, everolimus, gefitinib, imatinib, lapatinib, nilotinib, pazonanib, sorafenib, sunitinib, temsirolimus; Other Angiogenesis Inhibitors such as for example GT-111, JI-101, R1530; Other Kinase Inhibitors such as for example AC220, AC480, ACE-041, AMG 900, AP24534, Arry-614, AT7519, AT9283, AV-951, axitinib, AZD1152, AZD7762, AZD8055, AZD8931, bafetinib, BAY 73-4506, BGJ398, BGT226, BI 811283, BI6727, BIBF 1120, BIBW 2992, BMS-690154, BMS-777607, BMS-863233, BSK-461364, CAL-101, CEP-11981, CYC116, DCC-2036, dinaciclib, dovitinib lactate, E7050, EMD 1214063, ENMD-2076, fostamatinib disodium, GSK2256098, GSK690693, INCB18424, INNO-406, JNJ-26483327, JX-594, KX2-391, linifanib, LY2603618, MGCD265, MK-0457, MK1496, MLN8054, MLN8237, MP470, NMS-1116354, NMS-1286937, ON 01919.Na, OSI-027, OSI-930, Btk inhibitor, PF-00562271, PF-02341066, PF-03814735, PF-04217903, PF-04554878, PF-04691502, PF-3758309, PHA-739358, PLC3397, progenipoietin, R547, R763, ramucirumab, regorafenib, RO5185426, SAR103168, SCH 727965, SGI-1176, SGX523, SNS-314, TAK-593, TAK-901, TK1258, TLN-232, TTP607, XL147, XL228, XL281RO5126766, XL418, XL765.

Further examples of anti-cancer agents for use in combination with a Btk inhibitor compound include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

Other anti-cancer agents that can be employed in combination with a Btk inhibitor compound include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rIL2), interferon alfa-2a; interferon alfa-2b; interferon alfa-n1; interferon alfa-n3; interferon beta-1a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with a Btk inhibitor compound include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-such as for example growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with a Btk inhibitor compound include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of alkylating agents that can be employed in combination a Btk inhibitor compound include, but are not limited to, nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

Examples of anti-cancer agents which act by arresting cells in the G2-M phases due to stabilized microtubules and which can be used in combination with a Btk inhibitor compound include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-1AABE (Cytoskeleton/Mt. Sinai School of Medicine), A-204197 (Abbott), T-607 (Tularik, also known as T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

In some embodiments, the additional anti-cancer agent that is a Bcl-2 inhibitor.

In some embodiments, the additional anti-cancer agent is immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1, PD-1, CTLA-4, LAG3, or TIM3. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the immune checkpoint inhibitor is an inhibitor of PD-L2.

In some embodiments, the solvates are administered in combination with a CD20 inhibitor. Exemplary CD20 inhibitors include, but are not limited to, ibritumomab tiuxetan, ofatumumab, rituximab, tositumomab, and obinutuzumab.

In some embodiments, the additional anticancer agent used in combination with the solvates described herein include CDK4 inhibitors (e.g., palbociclib).

In some embodiments, the additional cancer agent is a proteosome inhibitor. In some embodimentx, the proteasome inhibitor is selected from bortezomib or carfilzomib

In some embodiments, the additional cancer agent that can be administered in combination with the solvates is an HDAC inhibitor. In some embodiments, the HDAC inhibitor is abexinostat or a salt thereof. In some embodiments, the abexinostat or a salt thereof is abexinostat HCl. In some embodiments, the abexinostat or a salt thereof is abexinostat tosylate.

In some embodiments, the additional cancer agent that can be administered in combination with the solvates is a MALT1 inhibitor, MCL-1 inhibitor, IDH1 inhibitor, TLR inhibitor, or PIM inhibitor.

In some embodiments, the additional anti-cancer agent that can be administered in combination with the solvates is an immunomodulatory agent. Exemplary immunomodulatory agents include, but are not limited to, lenalidomide, thalidomide, and pomalidomide.

In some embodiments, the solvates are administered in combination with an additional agent selected from idelalisib (GS-1101), pentostatine and etopside. In some embodiments, the solvates are administered with an additional therapeutic agent comprising the HyperCVAD regimen (cyclophosphamide, vincristine, doxorubicin, dexamethasone alternating with methotrexate and cytarabine), FCR regimen (FCR (fludarabine, cyclophosphamide, rituximab), R-CHOP regimen (rituximab, cyclophosphamide, doxorubicin, vincristine, and prednisone), FCMR regimen (fludarabine, cyclophosphamide, mitoxantrone, rituximab), FMR regimen (fludarabine, mitoxantrone, rituximab), PCR regimen (pentostatin, cyclophosphamide, rituximab), PEPC regimen (prednisone, etoposide, procarbazine, cyclophosphamide), an autologous stem cell transplant, ⁹⁰Y-ibritumomab tiuxetan or ¹³¹I-tositumomab. In some embodiments, the HyperCVAD regimen is administered in combination with rituximab.

In some embodiments, the solvates may be used with an analgesic such as acetaminophen.

The solvates may be used in any combination with one or more other anti-thromboembolic agents to treat or prevent thromboembolic disorder (e.g., stroke). Examples of anti-thromboembolic agents include, but are not limited any of the following: thrombolytic agents (e.g., alteplase anistreplase, streptokinase, urokinase, or tissue plasminogen activator), heparin, tinzaparin, warfarin, dabigatran (e.g., dabigatran etexilate), factor Xa inhibitors (e.g., fondaparinux, draparinux, rivaroxaban, DX-9065a, otamixaban, LY517717, or YM150), ticlopidine, clopidogrel, CS-747 (prasugrel, LY640315), ximelagatran, or BIBR 1048.

Where the individual is suffering from or at risk of suffering from an autoimmune disease, an inflammatory disease, or an allergy disease, a solvate of Compound 1 can be used in with one or more of the following therapeutic agents in any combination: immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics.

### Kits/Articles of Manufacture

For use in the therapeutic methods of use described herein, kits and articles of manufacture are also described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Packaging materials for use in packaging pharmaceutical products include, e.g., U.S. Pat. No. 5,323,907 (incorporated by reference). Examples of pharmaceutical packaging materials include, but are not limited to, blister packs, bottles, tubes, bags, containers, bottles, and any packaging material suitable for a selected formulation and intended mode of administration and treatment.

In some embodiments, the solvates of Compound 1 or compositions described herein, are presented in a package or dispenser device which may contain one or more unit dosage forms containing the active ingredient. The solvate of Compound 1 or composition described herein is packaged alone, or packaged with another compound or another ingredient or additive. In some embodiments, the package contains one or more containers filled with one or more of the ingredients of the pharmaceutical compositions. In some embodiments, the package comprises metal or plastic foil, such as a blister pack. In some embodiments, the package or dispenser device is accompanied by instructions for administration, such as instructions for administering the solvates of Compound 1 or compositions for treating a neoplastic disease. In some embodiments, the package or dispenser is accompanied with a notice associated with the container in form prescribed by a governmental agency regulating the manufacture, use, or sale of pharmaceuticals, which notice is reflective of approval by the agency of the form of the drug for human or veterinary administration. In some embodiments, such notice, for example, is the labeling approved by the U.S. Food and Drug Administration for prescription drugs, or the approved product insert. In some embodiments, compositions include a solvate of Compound 1 described herein formulated in a compatible pharmaceutical carrier are prepared, placed in an appropriate container, and labeled for treatment of an indicated condition.

For example, the container(s) include a solvate of Compound 1, optionally in a composition or in combination with another agent as disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a solvate of Compound 1 provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Examples

The following ingredients, formulations, processes and procedures for practicing the methods disclosed herein correspond to that described above.

### Example 1: Preparation of Amporphous 1-((R)-3-(4-amino-3-(4-phenoxyphenyl)-1H-pyrazolo[3,4-d]pyrimidin-1-yl)piperidin-1-yl)prop-2-en-1-one (Compound 1)

Compound 1 was dissolved in DCM (20 volumes) and filtered to remove any remaining solid particles. The solvent was then removed under vacuum (30° C., 200 mm Hg). The solids generated were analyzed by XRPD.

### Example 2a: Preparation of Crystalline Forms of Compound 1 Solvates—Maturation Method

Twenty-eight vials each containing a suspension of amorphous Compound 1 (30 mg) and one of twenty-eight separate solvents (150 μL of acetic acid, acetophenone, benzonitrile, benzyl alcohol, 1-butanol, t-butanol, butyronitrile, chlorobenzene, chlorobenzotrifluoride, chloroform, cyclopentylmethyl ether, cyclohexane, cyclohexanone, 1,2-dichlorobenzene, 1,2-dichloroethane, 1,2-dimethoxyethane, dimethylacetamide, ethylene glycol, glycerol, hexafluorobenzene, dimethyl carbonate, methyl-THF, N-methylpyrrolidone, perfluorohexane, propionitrile, trifluoroethanol, trifluorotoluene, or xylene] were stirred at 5° C. overnight. Vials containing suspensions were filtered and the collected solid was analyzed by XRPD. Vials containing solutions were allowed to slowly evaporate at room temperature and the remaining solid was analyzed by XRPD. Using this procedure, the following crystalline Compound 1 solvates were obtained: Compound 1 butyronitrile solvate (Form 1), Compound 1 1,2-dimethoxyethane solvate (Form 2), Compound 1 hexafluorobenzene solvate (Form 3), Compound 1 acetophenone solvate (Form 5), and Compound 1 chlorobenzene solvate (Form 6).

### Example 2b: Preparation of Crystalline Forms of Compound 1 Solvates—Maturation Method

Twenty-eight vials each containing a suspension of amorphous Compound 1 (30 mg) and one of twenty-eight separate solvents (150 μL of acetic acid, acetophenone, benzonitrile, benzyl alcohol, 1-butanol, t-butanol, butyronitrile, chlorobenzene, chlorobenzotrifluoride, chloroform, cyclopentylmethyl ether, cyclohexane, cyclohexanone, 1,2-dichlorobenzene, 1,2-dichloroethane, 1,2-dimethoxyethane, dimethylacetamide, ethylene glycol, glycerol, hexafluorobenzene, dimethyl carbonate, methyl-THF, N-methylpyrrolidone, perfluorohexane, propionitrile, trifluoroethanol, trifluorotoluene, or xylene] were stirred at 25° C. overnight. Vials containing suspensions were filtered and the collected solid was analyzed by XRPD. Vials containing solutions were allowed to evaporate at room temperature and the remaining solid was analyzed by XRPD. Using this procedure, the following crystalline Compound 1 solvates were obtained: Compound 1 butyronitrile solvate (Form 1), Compound 1 1,2-dimethoxyethane solvate (Form 2), Compound 1 hexafluorobenzene solvate (Form 4), Compound 1 acetophenone solvate (Form 5), and Compound 1 dimethylacetamide solvate (Form 8).

### Example 2c: Preparation of Crystalline Forms of Compound 1 Solvates—Maturation Method

Twenty-eight vials each containing a suspension of amorphous Compound 1 (30 mg) and one of twenty-eight separate solvents (150 μL of acetic acid, acetophenone, benzonitrile, benzyl alcohol, 1-butanol, t-butanol, butyronitrile, chlorobenzene, chlorobenzotrifluoride, chloroform, cyclopentylmethyl ether, cyclohexane, cyclohexanone, 1,2-dichlorobenzene, 1,2-dichloroethane, 1,2-dimethoxyethane, dimethylacetamide, ethylene glycol, glycerol, hexafluorobenzene, dimethyl carbonate, methyl-THF, N-methylpyrrolidone, perfluorohexane, propionitrile, trifluoroethanol, trifluorotoluene, or xylene] were stirred at 50° C. overnight. Vials containing suspensions were filtered and the collected solid was analyzed by XRPD. Vials containing solutions were allowed to evaporate at 50° C. and the remaining solid was analyzed by XRPD. Using this procedure, Compound 1 acetophenone solvate (Form 5) was obtained.

### Example 3: Scale-Up Preparation of Crystalline Compound 1 Butyronitrile Solvate (Form 1)

A solution of amorphous Compound 1 (109 mg) in butyronitrile (0.5 mL) was seeded with Compound 1 butyronitrile solvate (Form 1) obtained from Example 2a. The resulting suspension was stirred at 5° C. for 10 minutes. The solid was collected by filtration and washed with butyronitrile (0.5 mL) to give Compound 1 butyronitrile solvate (Form 1) (1.8 equivalents of butyronitrile).

### Example 4: Scale-Up Preparation of Crystalline Compound 1 1,2-Dimethoxyethane Solvate (Form 2)

A solution of amorphous Compound 1 (99 mg) in 1,2-dimethoxyethane (0.5 mL) was seeded with Compound 1 1,2-dimethoxyethane solvate (Form 2) obtained from Example 2b. The resulting suspension was stirred at 5° C. for 10 minutes. The thick suspension was diluted with 1,2-dimethoxyethane (0.5 mL). The solid was collected by filtration and washed with 1,2-dimethoxyethane (0.5 mL) to give Compound 1 1,2-dimethoxyethane solvate (Form 2) (0.6 equivalents of 1,2-dimethoxyethane).

### Example 5: Scale-Up Preparation of Crystalline Compound 1 Hexafluorobenzene Solvate (Form 4)

A suspension of amorphous Compound 1 (103 mg) in hexafluorobenzene (0.5 mL) was seeded with Compound 1 hexafluorobenzene solvate (Form 4) obtained from Example 2b. The thick suspension was allowed to evaporate at ambient conditions to give Compound 1 hexafluorobenzene solvate (Form 4).

### Example 6a: Scale-up Preparation of Crystalline Compound 1 Acetophenone Solvate (Form 5)

A solution of amorphous Compound 1 (110 mg) in acetophenone (0.5 mL) was seeded with Compound 1 acetophenone solvate (Form 5) obtained from Example 2a. The resulting suspension was stirred at 5° C. for 1 hour. The solid was collected by filtration and washed with acetophenone (0.5 mL) to give Compound 1 acetophenone solvate (Form 5) (1 equivalent of acetophenone).

### Example 6b: Scale-up Preparation of Crystalline Compound 1 Acetophenone Solvate (Form 5)

A slurry resulting from dissolving amorphous Compound 1 (2.005 g) in acetophenone (8 mL) was seeded with Compound 1 acetophenone solvate (Form 5) obtained from Example 2a. The resulting suspension was stirred at room temperature for 10 minutes and then at 5° C. for 2 hours. The solid was collected by filtration and washed with heptane (2×5 mL) to give Compound 1 acetophenone solvate (Form 5).

### Example 7a: Scale-up Preparation of Crystalline Compound 1 Chlorobenzene Solvate (Form 6)

A solution of amorphous Compound 1 (110 mg) in chlorobenzene (0.5 mL) was seeded with Compound 1 chlorobenzene solvate (Form 6) obtained from Example 2a. The resulting suspension was stirred at 5° C. for 1 hour. The solid was collected by filtration and washed with chlorobenzene (0.5 mL) to give Compound 1 chlorobenzene solvate (Form 6) (0.9 equivalents of chlorobenzene).

### Example 7b: Scale-up Preparation of Crystalline Compound 1 Chlorobenzene Solvate (Form 6)

A suspension of amorphous Compound 1 (250 mg) in chlorobenzene (1.2 mL) was seeded with Compound 1 chlorobenzene solvate (Form 6) obtained from Example 2a. The resulting suspension was stirred at 5° C. for 1 hour. The solid was collected by filtration to give Compound 1 chlorobenzene solvate (Form 6).

### Example 7c: Scale-up Preparation of Crystalline Compound 1 Chlorobenzene Solvate (Form 6)

A suspension of amorphous Compound 1 (1.002 g) in chlorobenzene (5 mL) was seeded with Compound 1 chlorobenzene solvate (Form 6) obtained from Example 2a. The resulting suspension was stirred at room temperature for 10 minutes and then stirred at 5° C. for 2 hours. The solid was collected by filtration and washed with chlorobenzene (2 mL) to give Compound 1 chlorobenzene solvate (Form 6).

### Example 8: Scale-Up Preparation of Crystalline Compound 1 Acetophenone Solvate (Form 7)

A suspension of Compound 1 acetophenone solvate (Form 5) obtained from Example 6b (300 mg) in heptane (3 mL) was sonicated at room temperature for 20 minutes. The solid was collected by filtration and washed with heptane (3 mL). The solid was re-suspended in heptane and stirred at room temperature for 72 hours. The solid was collected by filtration to give Compound 1 acetophenone solvate (Form 7) (0.46 equivalent of acetophenone).

### Example 9: Scale-Up Preparation of Crystalline Compound 1 Dimethylacetamide Solvate (Form 8)

A suspension of amorphous Compound 1 (250 mg) in dimethylacetamide (0.3 mL) was stirred at 50° C. The solution (open vial) was stirred overnight at room temperature. After drying for 48 hours at ambient conditions, the solid was placed in a vacuum oven at 25° C. overnight (about 16 hours) to give Compound 1 dimethylacetamide solvate (Form 8).

### Example 10: Preparation of Crystalline Compound 1 Benzyl Acetate Solvate (Form 9)

Amorphous Compound 1 (about 40 mg) was suspended in benzyl acetate (800 μL, 20 vol.) and the resulting mixture was left stirring overnight at 25° C. The resulting suspension was filtered to give Compound 1 benzyl acetate solvate (Form 9) (0.5 equivalent of benzyl acetate).

### Example 11: Preparation of Crystalline Compound 1 1,1,2-Trichlorethane Solvate (Form 10)

Compound 1 (144 mg) was dissolved in 1,1,2-trichlorethane (720 ptL) at 50° C. and treated with heptane (3 mL). The resulting suspension became a gel; the biphasic mixture was placed in the fridge at 5° C. for 30 min. The solid was filtered and air dried for 10 minutes to give Compound 1 1,1,2-trichlorethane solvate (Form 10) (124.8 mg, yield about 87%) (0.34 equivalent of 1,1,2-trichlorethane, 0.11 eq of heptane).

### Example 12: X-Ray Powder Diffraction (XRPD)

X-Ray powder diffraction patterns were collected on a Bruker AXS C2 GADDS or Bruker AXS D8 diffractometer.

Bruker AXS C2 GADDS

X-Ray Powder Diffraction patterns were collected on a Bruker AXS C2 GADDS diffractometer using Cu Ka radiation (40 kV, 40 mA), automated XYZ stage, laser video microscope for auto-sample positioning and a HiStar 2-dimensional area detector. X-ray optics consists of a single Gobel multilayer mirror coupled with a pinhole collimator of 0.3 mm. A weekly performance check is carried out using a certified standard NIST 1976 Corundum (flat plate). The beam divergence, i.e. the effective size of the X-ray beam on the sample, was approximately 4 mm. A θ-θ continuous scan mode was employed with a sample-detector distance of 20 cm which gives an effective 20 range of 3.2°-29.7°. Typically the sample would be exposed to the X-ray beam for 120 seconds. The software used for data collection was GADDS for XP/2000 4.1.43 and the data were analysed and presented using Diffrac Plus EVA v15.0.0.0.

### Ambient Conditions

Samples run under ambient conditions were prepared as flat plate specimens using powder as received without grinding. Approximately 1-2 mg of the sample was lightly pressed on a glass slide to obtain a flat surface.

### Non-Ambient Conditions

Samples run under non-ambient conditions were mounted on a silicon wafer with heatconducting compound. The sample was then heated to the appropriate temperature at 10° C./min (unless otherwise stated) and subsequently held isothermally for 1 minute before data collection was initiated.

Bruker AXS D8 Advance

X-Ray Powder Diffraction patterns were collected on a Bruker D8 diffractometer using Cu Ka radiation (40 kV, 40 mA), 0-20 goniometer, and divergence of V4 and receiving slits, a Ge monochromator and a Lynxeye detector. The instrument is performance checked using a certified Corundum standard (NIST 1976). The software used for data collection was Diffrac Plus XRD Commander v2.6.1 and the data were analysed and presented using Diffrac Plus EVA v15.0.0.0. Samples were run under ambient conditions as flat plate specimens using powder as received. The sample was gently packed into a cavity cut into polished, zero-background (510) silicon wafer. The sample was rotated in its own plane during analysis. The details of the data collection are:
Angular range: 2 to 42° 20
Step size: 0.05° 20
Collection time: 0.5 s/step

XRPD of Compound 1 Butyronitrile Solvate (Form 1)

The X-ray powder diffraction for Compound 1 butyronitrile solvate (Form 1) is displayed in FIG. 1. Characteristic peaks include 2.7±0.1° 2-Theta, 5.5±0.1° 2-Theta, 10.9±0.1° 2-Theta, 13.6±0.1° 2-Theta, 14.8±0.1° 2-Theta, 17.3±0.1° 2-Theta, 18.7±0.1° 2-Theta, 20.0±0.1° 2-Theta, and 21.8±0.1° 2-Theta. In one example, the X-ray powder diffraction for Compound 1 butyronitrile solvate has the peaks in Table 1:

TABLE 1

| Angle 2-Theta ° | Intensity % |
|---|---|
| 2.69 | 24.8 |
| 5.45 | 100.0 |
| 10.86 | 3.9 |
| 13.57 | 4.7 |
| 14.83 | 3.6 |
| 17.33 | 3.9 |
| 18.66 | 12.1 |
| 19.98 | 10.1 |
| 21.80 | 5.4 |

XRPD of Compound 1 1,2-Dimethoxyethane Solvate (Form 2)

The X-ray powder diffraction for Compound 1 1,2-dimethoxyethane solvate (Form 2) is displayed in FIG. 3. Characteristic peaks include 6.88±0.1° 2-Theta, 13.4±0.10 2-Theta, 17.6±0.1° 2-Theta, 18.2±0.1° 2-Theta, 20.2±0.1° 2-Theta, 21.2±0.1° 2-Theta, and 22.2±0.1° 2-Theta. In one example, the X-ray powder diffraction for Compound 1 dimethoxyethane solvate has the peaks in Table 2:

TABLE 2

| Angle 2-Theta ° | Intensity % |
|---|---|
| 6.75 | 100.0 |
| 10.30 | 5.5 |
| 10.70 | 9.2 |
| 13.41 | 74.6 |
| 15.49 | 12.7 |
| 16.78 | 6.2 |
| 17.35 | 18.6 |
| 17.62 | 35.0 |
| 18.23 | 37.0 |
| 18.60 | 6.4 |
| 18.92 | 28.3 |
| 20.18 | 34.2 |
| 20.52 | 29.0 |
| 21.18 | 38.4 |
| 21.45 | 24.3 |
| 21.70 | 7.0 |
| 22.15 | 53.5 |
| 23.13 | 7.1 |
| 23.37 | 22.4 |
| 23.58 | 17.3 |
| 27.01 | 17.5 |
| 29.74 | 15.9 |
| 30.04 | 8.5 |
| 30.70 | 6.2 |
| 34.00 | 6.2 |

Crystallinity was unaffected after one week storage at 40° C./75% RH.

XRPD of Compound 1 Hexafluorobenzene Solvate (Form 3)

The X-ray powder diffraction for Compound 1 hexafluorobenzene solvate (Form 3) is displayed in FIG. 5. Characteristic peaks include 5.4±0.1° 2-Theta, 14.0±0.1° 2-Theta, 16.1±0.1° 2-Theta, 18.6±0.1° 2-Theta, 19.3±0.1° 2-Theta, 22.4±0.1° 2-Theta, and 23.6±0.10 2-Theta. In one example, the X-ray powder diffraction for Compound 1 hexafluorobenzene solvate has the peaks in Table 3:

TABLE 3

| Angle 2-Theta ° | Intensity % |
|---|---|
| 5.37 | 100.0 |
| 7.66 | 21.7 |
| 9.78 | 20.1 |
| 10.69 | 15.4 |
| 13.98 | 40.1 |
| 16.07 | 57.4 |
| 17.69 | 23.7 |
| 18.57 | 33.5 |
| 19.25 | 39.3 |
| 20.06 | 21.4 |
| 22.42 | 47.3 |
| 23.55 | 32.8 |

XRPD of Compound 1 Hexafluorobenzene Solvate (Form 4)

The X-ray powder diffraction for Compound 1 hexafluorobenzene solvate (Form 4) is displayed in FIG. 7. Characteristic peaks include 12.6±0.1° 2-Theta, 15.4±0.1° 2-Theta, 17.7±0.1° 2-Theta, 24.9±0.1° 2-Theta, 25.4±0.1° 2-Theta, and 26.9±0.1° 2-Theta. In one example, the X-ray powder diffraction for Compound 1 hexafluorobenzene solvate has the peaks in Table 4:

TABLE 4

| Angle 2-Theta ° | Intensity % |
|---|---|
| 5.25 | 7.1 |
| 6.81 | 7.2 |
| 8.75 | 9.7 |
| 10.16 | 9.2 |
| 12.58 | 37.8 |
| 13.38 | 14.4 |
| 14.70 | 20.2 |
| 15.36 | 75.5 |
| 16.25 | 12.8 |
| 16.70 | 7.3 |
| 17.18 | 8.6 |
| 17.65 | 25.6 |
| 17.88 | 20.4 |
| 18.25 | 6.9 |
| 18.80 | 12.0 |
| 19.32 | 9.2 |
| 19.95 | 9.6 |
| 20.43 | 18.4 |
| 20.85 | 10.1 |
| 21.11 | 11.4 |
| 22.67 | 14.2 |
| 22.96 | 12.5 |
| 23.89 | 20.4 |
| 24.86 | 27.5 |
| 25.44 | 100.0 |
| 26.37 | 23.5 |
| 26.92 | 32.5 |
| 28.68 | 8.0 |
| 29.25 | 8.3 |
| 29.60 | 14.9 |

XRPD of Compound 1 Acetophenone Solvate (Form 5)

The X-ray powder diffraction for Compound 1 acetophenone solvate (Form 5) is displayed in FIG. 9. Characteristic peaks include 7.6±0.1° 2-Theta, 8.8±0.1° 2-Theta, 15.2±0.1° 2-Theta, 17.6±0.1° 2-Theta, 18.9±0.1° 2-Theta, 19.5±0.1° 2-Theta, 20.4±0.1° 2-Theta, 21.0±0.1° 2-Theta, 21.3±0.1° 2-Theta, 21.8±0.1° 2-Theta, 24.3±0.1° 2-Theta, and 24.8±0.1° 2-Theta. In one example, the X-ray powder diffraction for Compound 1 acetophenone solvate has the peaks in Table 5:

TABLE 5

| Angle 2-Theta ° | Intensity % |
|---|---|
| 7.60 | 46.6 |
| 8.01 | 9.5 |
| 8.76 | 23.4 |
| 10.17 | 14.6 |
| 13.08 | 12.2 |
| 14.03 | 6.6 |
| 14.86 | 9.1 |
| 15.17 | 47.0 |
| 15.41 | 20.7 |
| 15.80 | 9.4 |
| 16.55 | 17.3 |
| 17.14 | 8.0 |
| 17.59 | 24.5 |
| 18.38 | 20.2 |
| 18.85 | 99.8 |
| 19.30 | 18.1 |
| 19.49 | 50.1 |
| 20.43 | 100.0 |
| 20.97 | 54.7 |
| 21.25 | 40.5 |
| 21.82 | 46.1 |
| 22.65 | 22.1 |
| 23.56 | 10.7 |
| 24.31 | 66.0 |
| 24.82 | 30.1 |
| 25.80 | 10.9 |
| 26.04 | 9.4 |
| 26.59 | 9.4 |
| 27.66 | 16.2 |
| 28.26 | 10.0 |
| 28.70 | 9.9 |
| 29.06 | 15.1 |
| 29.54 | 9.9 |
| 30.82 | 10.8 |
| 32.82 | 8.7 |
| 33.62 | 10.4 |

XRPD of Compound 1 Chlorobenzene Solvate (Form 6)

The X-ray powder diffraction for Compound 1 chlorobenzene solvate (Form 6) is displayed in FIG. 11. Characteristic peaks include 18.4±0.1° 2-Theta, 19.4±0.1° 2-Theta, 20.2±0.1° 2-Theta, 20.9±0.1° 2-Theta, 21.2±0.1° 2-Theta, 21.9±0.1° 2-Theta, and 25.0±0.10 2-Theta. In one example, the X-ray powder diffraction for Compound 1 chlorobenzene solvate has the peaks in Table 6:

TABLE 6

| Angle 2-Theta ° | Intensity % |
|---|---|
| 7.64 | 21.6 |
| 7.91 | 5.8 |
| 8.86 | 15.8 |
| 10.55 | 9.4 |
| 12.99 | 8.6 |
| 13.77 | 5.9 |
| 15.01 | 6.3 |
| 15.29 | 15.8 |
| 15.73 | 21.3 |
| 16.89 | 7.5 |
| 17.73 | 23.7 |
| 18.13 | 10.4 |
| 18.43 | 38.8 |
| 19.39 | 100.0 |
| 20.23 | 36.0 |
| 20.92 | 44.1 |
| 21.20 | 31.8 |
| 21.52 | 27.8 |
| 21.85 | 36.9 |
| 22.57 | 20.4 |
| 24.02 | 18.9 |
| 24.45 | 8.1 |
| 24.80 | 15.4 |
| 25.04 | 39.2 |
| 25.59 | 12.3 |
| 25.84 | 9.6 |
| 26.70 | 8.7 |
| 27.82 | 14.1 |
| 28.46 | 10.1 |
| 28.98 | 12.1 |

Crystallinity was unaffected after one week storage at 40° C./75% RH.

XRPD of Compound 1 Acetophenone Solvate (Form 7)

The X-ray powder diffraction for Compound 1 acetophenone solvate (Form 7) is displayed in FIG. 13. Characteristic peaks include 6.5±0.1° 2-Theta, 13.0±0.1° 2-Theta, 17.6±0.1° 2-Theta, 18.4±0.1° 2-Theta, 19.9±0.1° 2-Theta, 21.0±0.1° 2-Theta, 21.5±0.1° 2-Theta, 22.1±0.1° 2-Theta, and 23.9±0.1° 2-Theta. In one example, the X-ray powder diffraction for Compound 1 acetophenone solvate has the peaks in Table 7:

TABLE 7

| Angle 2-Theta ° | Intensity % |
|---|---|
| 6.49 | 29.3 |
| 9.58 | 24.7 |
| 10.05 | 7.4 |
| 10.41 | 22.8 |
| 12.37 | 14.1 |
| 12.95 | 39.8 |
| 14.23 | 21.2 |
| 16.96 | 20.6 |
| 17.62 | 81.7 |
| 17.87 | 35.3 |
| 18.36 | 73.5 |
| 19.27 | 26.7 |
| 19.91 | 100.0 |
| 20.25 | 22.4 |
| 20.95 | 74.3 |
| 21.48 | 51.5 |
| 22.09 | 44.5 |
| 23.26 | 31.3 |
| 23.87 | 42.1 |
| 25.23 | 21.8 |
| 25.85 | 28.4 |
| 27.36 | 26.4 |
| 28.10 | 9.4 |
| 28.34 | 16.1 |
| 28.79 | 28.1 |
| 29.66 | 10.9 |
| 30.44 | 8.9 |
| 31.11 | 9.4 |
| 31.91 | 9.5 |
| 32.37 | 7.8 |
| 41.50 | 8.8 |

XRPD of Compound 1 Dimethylacetamide Solvate (Form 8)

The X-ray powder diffraction for Compound 1 dimethylacetamide solvate (Form 8) is displayed in FIG. 15. Characteristic peaks include 8.8±0.1° 2-Theta, 19.2±0.1° 2-Theta, 19.9±0.1° 2-Theta, 22.5±0.1° 2-Theta, 24.5±0.1° 2-Theta, and 25.3±0.1° 2-Theta. In one example, the X-ray powder diffraction for Compound 1 dimethylacetamide solvate has the peaks in Table 8:

TABLE 8

| Angle 2-Theta ° | Intensity % |
|---|---|
| 6.37 | 6.1 |
| 8.80 | 32.9 |
| 9.36 | 20.4 |
| 9.88 | 12.5 |
| 10.84 | 9.4 |
| 11.38 | 14.1 |
| 12.65 | 14.5 |
| 15.89 | 5.7 |
| 16.56 | 17.6 |
| 17.38 | 23.4 |
| 17.64 | 25.0 |
| 18.41 | 24.0 |
| 19.15 | 55.2 |
| 19.87 | 100.0 |
| 20.11 | 16.7 |
| 21.40 | 7.1 |
| 21.79 | 26.1 |
| 22.50 | 72.8 |
| 22.99 | 19.5 |
| 23.83 | 15.1 |
| 24.25 | 16.9 |
| 24.48 | 26.6 |
| 25.30 | 30.0 |
| 26.23 | 11.0 |
| 26.66 | 7.7 |
| 27.34 | 11.2 |
| 28.41 | 12.4 |
| 29.06 | 7.3 |
| 30.05 | 9.6 |

XRPD of Compound 1 Benzyl Acetate Solvate (Form 9)

The X-ray powder diffraction for Compound 1 benzyl acetate solvate (Form 9) is displayed in FIG. 17. Characteristic peaks include 12.8±0.1° 2-Theta, 17.8±0.1° 2-Theta, 18.7±0.1° 2-Theta, 19.2±0.1° 2-Theta, 20.1±0.1° 2-Theta, 20.7±0.1° 2-Theta, 22.1±0.1° 2-Theta and 22.9±0.1° 2-Theta. In one example, the X-ray powder diffraction for Compound 1 benzyl acetate solvate has the peaks in Table 9:

TABLE 9

| Angle 2-Theta ° | Intensity % |
|---|---|
| 6.3 | 26.8 |
| 9.7 | 14.7 |
| 10.0 | 18.2 |
| 12.5 | 21.1 |
| 12.8 | 66.1 |
| 13.4 | 10.6 |
| 17.2 | 15.4 |
| 17.3 | 24.8 |
| 17.8 | 43.2 |
| 18.7 | 86.5 |
| 19.2 | 100.0 |
| 19.5 | 14.3 |
| 20.1 | 67.7 |
| 20.4 | 14.2 |
| 20.7 | 47.1 |
| 21.8 | 39.3 |
| 22.1 | 48.5 |
| 22.9 | 43.4 |
| 23.5 | 14.2 |
| 24.4 | 12.2 |
| 25.0 | 10.3 |
| 26.1 | 18.5 |
| 26.7 | 12.6 |
| 27.5 | 22.6 |
| 27.7 | 20.6 |
| 28.6 | 6.6 |
| 29.6 | 7.6 |
| 30.2 | 11.5 |
| 30.6 | 9.1 |
| 32.1 | 6.7 |
| 33.7 | 4.8 |
| 34.7 | 5.3 |

XRPD of Compound 1 1,1,2-Trichloroethane Solvate (Form 10)

The X-ray powder diffraction for Compound 1 1,1,2-trichloroethane solvate (Form 10) is displayed in FIG. 19. Characteristic peaks include 5.4±0.1° 2-Theta, 18.6±0.10 2-Theta, 20.1±0.1° 2-Theta, 20.8±0.1° 2-Theta, 21.3±0.1° 2-Theta, 21.7±0.1° 2-Theta, and 22.6±0.1° 2-Theta. In one example, the X-ray powder diffraction for Compound 1 1,1,2-trichloroethane solvate has the peaks in Table 10:

TABLE 10

| Angle 2-Theta ° | Intensity % |
|---|---|
| 5.4 | 83.8 |
| 10.5 | 30.7 |
| 10.8 | 24.2 |
| 11.9 | 15.4 |
| 12.5 | 15.4 |
| 13.3 | 22.4 |
| 15.0 | 31.8 |
| 15.7 | 37.2 |
| 16.1 | 24.5 |
| 17.3 | 33.3 |
| 18.6 | 66.7 |
| 19.2 | 30.6 |
| 19.6 | 34.8 |
| 20.1 | 70.8 |
| 20.8 | 40.4 |
| 21.3 | 100.0 |
| 21.7 | 59.3 |
| 22.6 | 41.9 |
| 24.4 | 26.4 |
| 25.8 | 28.9 |
| 26.3 | 29.6 |
| 28.9 | 19.8 |

### Example 13: Single Crystal X-Ray Diffraction of Compound 1 Acetophenone Solvate (Form 5)

Single crystal X-ray diffraction data was collected and processed as follows:

| | |
|---|---|
| Diffractometer | SuperNova, Dual, Cu at zero, Atlas |
| Radiation source | SuperNova (Cu) X-ray Source, CuKα |
| Data collection method | Omega scans |
| Theta range for data collection | 3.851 to 76.274° |
| Index ranges | −14 ≤ h ≤ 12, −14 ≤ k ≤ 14, |
| | −15 ≤ l ≤ 15 |
| Reflections collected | 29536 |
| Independent reflections | 10931 [R(int) = 0.0358] |
| Coverage of independent | 100.0% |
| reflections | |
| Variation in check reflections | N/A |
| Absorption correction | Semi-empirical from equivalents |
| Max. and min. transmission | 1.00000 and 0.53806 |
| Structure solution technique | Direct Methods |
| Structure solution program | SHELXTL (Sheldrick, 2013) |
| Refinement technique | Full-matrix least-squares on F² |
| Refinement program | SHELXTL (Sheldrick, 2013) |
| Function minimized | Σw(Fₒ² − F_{c}²)² |
| Data/restraints/parameters | 10931/3/858 |
| Goodness-of-fit on F² | 1.044 |
| Δ/σₘₐₓ | 0.000 |
| Final R indices | |
| 10485 data; I > 2σ(I) | R1 = 0.0454, wR2 = 0.1175 |
| all data | R1 = 0.0476, wR2 = 0.1208 |
| Weighting scheme | w = 1/[σ² (Fₒ²) + (0.0716P)² + 0.3711P] |
| | where P = (Fₒ² + 2F_{c}²)/3 |
| Absolute structure parameter | 0.05(9) |
| Extinction coefficient | n/a |
| Largest diff. peak and hole | 0.354 and −0.286 eÅ⁻³ |

Compound 1 acetophenone solvate (Form 5) was characterized by unit cell parameters approximately equal to the following at a temperature of approximately 100(2) K:

| Molecular formula | C₃₃H₃₂N₆O₃ | | | | |
|---|---|---|---|---|---|
| Molecular weight | 560.64 | | | | |
| Crystal system | Triclinic | | | | |
| Space group | P1 | a | 11.3552(5) Å | α | 79.657(3)° |
| | | b | 11.7741(4) Å | β | 70.352(4)° |
| | | c | 12.2064(4) Å | γ | 67.080(4)° |

| | |
|---|---|
| V | 1413.38(11) Å³ |
| Z | 2 |
| Density (calculated) | 1.317 Mg/m³ |
| Absorption coefficient | 0.699 mm⁻¹ |
| Wavelength | 1.54178 Å |
| F(000) | 592 |
| T | 100(2) K |

### Example 14: Single Crystal X-Ray Diffraction of Compound 1 Dimethylacetamide Solvate (Form 8)

Single crystal X-ray diffraction data was collected and processed as follows:

DiffractometerSuperNova, Dual, Cu at zero, Atlas

| | |
|---|---|
| Radiation source | SuperNova (Cu) X-ray Source, CuKα |
| Data collection method | Omega scans |
| Theta range for data collection | 8.913 to 74.496° |
| Index ranges | −11 ≤ h ≤ 11, −13 ≤ k ≤ 12, |
| | −18 ≤ l ≤ 18 |
| Reflections collected | 26862 |
| Independent reflections | 10221 [R(int) = 0.0297] |
| Coverage of independent | 99.6% |
| reflections | |
| Variation in check reflections | n/a |
| Absorption correction | Semi-empirical from equivalents |
| Max. and min. transmission | 1.00000 and 0.47869 |
| Structure solution technique | Direct methods |
| Structure solution program | SHELXTL (Sheldrick, 2013) |
| Refinement technique | Full-matrix least-squares on F² |
| Refinement program | SHELXTL (Sheldrick, 2013) |
| Function minimized | Σ w(Fₒ² − F_{c}²) |
| Data/restraints/parameters | 10221/3/783 |
| Goodness-of-fit on F² | 1.029 |
| Δ/σₘₐₓ | 0.000 |
| Final R indices | |
| 9847 data; I > 2σ(I) | R1 = 0.0342, wR2 = 0.0861 |
| all data | R1 = 0.0361, wR2 = 0.0881 |
| Weighting scheme | w = 1/[σ² (Fₒ²) + (0.0486P)² + 0.2940P] |
| | where P = (Fₒ² + 2F_{c}²)/3 |
| Absolute structure parameter | 0.01(7) |
| Extinction coefficient | n/a |
| Largest diff. peak and hole | 0.629 and −0.281 eÅ⁻³ |

Compound 1 dimethylacetamide solvate (Form 8) was characterized by unit cell parameters approximately equal to the following at a temperature of approximately 100(2) K:

| Molecular formula | C₂₉H₃₃N₇O₃ | | | | |
|---|---|---|---|---|---|
| Molecular weight | 527.62 | | | | |
| Crystal system | Triclinic | | | | |
| Space group | P1 | a | 9.3627(3) Å | α | 70.831(3)° |
| | | b | 10.9543(4) Å | β | 76.034(3)° |
| | | c | 14.7742(5) Å | γ | 70.721(3)° |

| | |
|---|---|
| V | 1335.88(9) Å³ |
| Z | 2 |
| Density (calculated) | 1.312 Mg/m³ |
| Absorption coefficient | 0.711 mm⁻¹ |
| Wavelength | 1.54178 Å |
| F(000) | 560 |
| T | 100(2) K |

### Example 15: Differential Scanning Calorimetry (DSC) and Thermo-Gravimetric Analysis (TGA)

DSC data were collected on a TA Instruments Q2000 equipped with a 50 position autosampler. The calibration for thermal capacity was carried out using sapphire and the calibration for energy and temperature was carried out using certified indium. Typically 0.5-3 mg of each sample, in a pin-holed aluminium pan, was heated at 10° C./min from 25° C. to 300° C. A purge of dry nitrogen at 50 mL/min was maintained over the sample, unless otherwise stated. Modulated temperature DSC was carried out using an underlying heating rate of 2° C./min and temperature modulation parameters of ±0.636° C. (amplitude) every 60 seconds (period). The instrument control software was Advantage for Q Series v2.8.0.394 and Thermal Advantage v5.5.3 and the data were analysed using Universal Analysis v4.5A.

TGA data were collected on a TA Instruments Q500 TGA, equipped with a 16 position autosampler. The instrument was temperature calibrated using certified Alumel and Nickel. Typically 5-10 mg of each sample was loaded onto a pre-tared aluminium DSC pan and heated at 10° C./min from ambient temperature to 350° C. A nitrogen purge at 60 mL/min was maintained over the sample, unless otherwise stated. The instrument control software was Advantage for Q Series v2.5.0.256 and Thermal Advantage v4.8.3 and the data were analysed using Universal Analysis v4.5A.

Compound 1 Butyronitrile Solvate (Form 1)

DSC and TGA thermograms for Compound 1 Butyronitrile Solvate (Form 1) are displayed in FIG. 2.

Loss of about 14.4% w/w between about 25-80° C. Loss of about 3.7% w/w between about 100-140° C.

In the DSC (rate of heating: 10° C./min or 20° C./min), a broad endotherm was observed between about 25-80° C. and another endotherm between about 100-125° C. The DSC further has endotherm onset at about 153° C. and a peak at about 156° C.

Compound 1 1,2-Dimethoxyethane Solvate (Form 2)

DSC and TGA thermograms for Compound 1 1,2-Dimethoxyethane Solvate (Form 2) are displayed in FIG. 4.

Loss of about 4.0% w/w between about 60-110° C.

In the DSC (rate of heating: 10° C./min or 20° C./min), endotherm onset at about 89° C. (e.g., 89-93° C.) and a peak at about 101° C.

Compound 1 Hexafluorobenzene Solvate (Form 3)

DSC thermogram for Compound 1 Hexafluorobenzene Solvate (Form 3) is displayed in FIG. 6.

In the DSC (rate of heating: 10° C./min or 20° C./min), a broad double endotherm between about 51-100° C., and a small endotherm onset at about 152° C.

Compound 1 Hexafluorobenzene Solvate (Form 4)

DSC and TGA thermograms for Compound 1 Hexafluorobenzene Solvate (Form 4) are displayed in FIG. 8.

Loss of about 20.6% w/w between about 84-110° C.

In the DSC (rate of heating: 10° C./min or 20° C./min), a broad endotherm between about 84-110° C. with endotherm onset at about 84° C. and a peak at about 100° C.

Compound 1 Acetophenone Solvate (Form 5)

DSC and TGA thermograms for Compound 1 Acetophenone Solvate (Form 5) are displayed in FIG. 10.

A weight loss of about 22.6% w/w was observed between about 80-190° C.

In the DSC (rate of heating: 10° C./min or 20° C./min), endotherm onset at about 89° C. and a peak at about 96° C. The endotherm may be between about 50-110° C.

Compound 1 Chlorobenzene Solvate (Form 6)

DSC and TGA thermograms for Compound 1 Acetophenone Solvate (Form 5) are displayed in FIG. 12.

A weight loss of about 3.9% w/w was observed between about 75-95° C.

In the DSC (rate of heating: 10° C./min or 20° C./min), endotherm onset at about 92° C. and a peak at about 95° C.

Compound 1 Acetophenone Solvate (Form 7)

DSC and TGA thermograms for Compound 1 Acetophenone Solvate (Form 7) are displayed in FIG. 14.

A weight loss of about 11.5% w/w was observed between about 100-300° C.

In the DSC (rate of heating: 10° C./min or 20° C./min), endotherm onset at about 124° C. and a peak at about 127° C.

Compound 1 Dimethylacetamide Solvate (Form 8)

DSC and TGA thermograms for Compound 1 Dimethylacetamide Solvate (Form 8) are displayed in FIG. 16.

A weight loss of about 16.3% w/w was observed between about 50-300° C.

In the DSC (rate of heating: 10° C./min or 20° C./min), endotherm onset at about 82° C. and a peak at about 85° C.

Compound 1 Benzyl Acetate Solvate (Form 9)

DSC and TGA thermograms for Compound 1 benzyl acetate solvate (Form 9) are displayed in FIG. 18.

A weight loss of about 11.2% w/w between 25-140° C. and about 1.4% w/w between 140-170° C.

In the DSC (rate of heating: 10° C./min or 20° C./min), endotherm onset at about 105.7° C. and melt (onset) about 155.1° C.

Compound 1 1,1,2-Trichloroethane Solvate (Form 10)

DSC and TGA thermograms for Compound 1 1,1,2-trichloroethane solvate (Form 10) are displayed in FIG. 20.

A weight loss of about 6.8% w/w between 25-105° C.

In the DSC (rate of heating: 10° C./min or 20° C./min), broad endotherm at between about 55-100° C. and melt (onset) 150.3° C.

### Example 16: Safety and Tolerability Study of Compound 1 Solvate in Chronic Lymphocytic Leukemia

Purpose: The purpose of this study is to establish the safety and optimal dose of orally administered Compound 1 solvate (will be administered in an amount comprising 420 mg/day Compound 1) in patients with B-cell chronic lymphocytic leukemia/small lymphocytic lymphoma/diffuse well-differentiated lymphocytic lymphoma.

Primary Outcome Measures: Safety and tolerability of Compound 1 solvate (frequency, severity, and relatedness of adverse events).

Secondary Outcome Measures: Pharmacokinetic/Pharmacodynamic assessments. Tumor response—overall response rate as defined by recent guidelines on CLL and SLL (B cell lymphoma) and duration of response.

Eligibility: 18 Years and older; both genders are eligible.

Inclusion Criteria: 1. For treatment-naive group only: Men and women ≥65 years of age with confirmed diagnosis of CLL/SLL, who require treatment per NCI or International Working Group guidelines11-14. 2. For relapsed/refractory group only: Men and women ≥18 years of age with a confirmed diagnosis of relapsed/refractory CLL/SLL unresponsive to therapy (ie, failed ≥2 previous treatments for CLL/SLL and at least 1 regimen had to have had a purine analog [eg, fludarabine] for subjects with CLL). 3. Body weight ≥40 kg. 4. ECOG performance status of <2. 5. Agreement to use contraception during the study and for 30 days after the last dose of study drug if sexually active and able to bear children. 6. Willing and able to participate in all required evaluations and procedures in this study protocol including swallowing capsules without difficulty. 7. Ability to understand the purpose and risks of the study and provide signed and dated informed consent and authorization to use protected health information (in accordance with national and local subject privacy regulations).

Exclusion Criteria: 1. A life-threatening illness, medical condition or organ system dysfunction which, in the investigator's opinion, could compromise the subject's safety, interfere with the absorption or metabolism of Compound 1 solvate PO, or put the study outcomes at undue risk. 2. Any immunotherapy, chemotherapy, radiotherapy, or experimental therapy within 4 weeks before first dose of study drug (corticosteroids for disease-related symptoms allowed but require 1-week washout before study drug administration). 3. Central nervous system (CNS) involvement by lymphoma. 4. Major surgery within 4 weeks before first dose of study drug. 5. Creatinine >1.5×institutional upper limit of normal (ULN); total bilirubin >1.5×ULN (unless due to Gilbert's disease); and aspartate aminotransferase (AST) or alanine aminotransferase (ALT) >2.5×ULN unless disease related. 6. Concomitant use of medicines known to cause QT prolongation or torsades de pointes. 7. Significant screening electrocardiogram (ECG) abnormalities including left bundle branch block, 2nd degree AV block type II, 3rd degree block, bradycardia, and QTc>470 msec. 8. Lactating or pregnant.

### Example 17: Safety and Efficacy of Compound 1 Solvate in Subjects with Relapsed/Refractory Mantle Cell Lymphoma (MCL)

The primary objective of this trial is to evaluate the efficacy of Compound 1 solvate in relapsed/refractory subjects with Mantle Cell Lymphoma (MCL). The secondary objective is to evaluate the safety of a fixed daily dosing regimen of Compound 1 solvate (will be administered in an amount comprising 560 mg/day Compound 1 in the form of capsules) in this population.

Primary Outcome Measures: To measure the number of participants with a response to Compound 1 solvate.

Secondary Outcome Measures: To measure the number of participants with adverse events as a measure of safety and tolerability. To measure pharmacokinetics to assist in determining how the body responds to the study drug. Patient reported outcomes (to measure the number of participants reported outcomes in determining the health related quality of life).

Eligibility: 18 Years and older; both genders are eligible.

Inclusion Criteria: Men and women ≥18 years of age. ECOG performance status of ≤2. Pathologically confirmed MCL, with documentation of either overexpression of cyclin D1 or t(11;14), and measurable disease on cross sectional imaging that is ≥2 cm in the longest diameter and measurable in 2 perpendicular dimensions. Documented failure to achieve at least partial response (PR) with, or documented disease progression disease after, the most recent treatment regimen. At least 1, but no more than 5, prior treatment regimens for MCL (Note: Subjects having received ≥2 cycles of prior treatment with bortezomib, either as a single agent or as part of a combination therapy regimen, will be considered to be bortezomib-exposed.). Willing and able to participate in all required evaluations and procedures in this study protocol including swallowing capsules without difficulty. Ability to understand the purpose and risks of the study and provide signed and dated informed consent and authorization to use protected health information (in accordance with national and local subject privacy regulations).

Major exclusion criteria: Prior chemotherapy within 3 weeks, nitrosoureas within 6 weeks, therapeutic anticancer antibodies within 4 weeks, radio- or toxin-immunoconjugates within 10 weeks, radiation therapy within 3 weeks, or major surgery within 2 weeks of first dose of study drug. Any life-threatening illness, medical condition or organ system dysfunction which, in the investigator's opinion, could compromise the subject's safety, interfere with the absorption or metabolism of Compound 1 solvate capsules, or put the study outcomes at undue risk. Clinically significant cardiovascular disease such as uncontrolled or symptomatic arrhythmias, congestive heart failure, or myocardial infarction within 6 months of screening, or any Class 3 or 4 cardiac disease as defined by the New York Heart Association Functional Classification. Malabsorption syndrome, disease significantly affecting gastrointestinal function, or resection of the stomach or small bowel or ulcerative colitis, symptomatic inflammatory bowel disease, or partial or complete bowel obstruction. Any of the following laboratory abnormalities: 1. Absolute neutrophil count (ANC)<750 cells/mm3 (0.75×109/L) unless there is documented bone marrow involvement. 2. Platelet count <50,000 cells/mm3 (50×109/L) independent of transfusion support unless there is documented bone marrow involvement. 3. Serum aspartate transaminase (AST/SGOT) or alanine transaminase (ALT/SGPT)≥3.0×upper limit of normal (ULN). 4. Creatinine >2.0×ULN.

### Example 18: Phase 2 Study of the Combination of Compound 1 Solvate and Rituximab in High-Risk Chronic Lymphocytic Leukemia and Small Lymphocytic Lymphoma Patients

Purpose: The goal of this clinical research study is to learn if Compound 1 solvate combined with rituximab can help to control chronic lymphocytic leukemia (CLL) and small lymphocytic lymphoma (SLL). The safety of this combination will also be studied.

Rituximab (375 mg/m²) will be given intravenously (IV) on Day 1, Day 8, Day 15, and Day 22, then continued once every 4 weeks only on Days 1 during cycles 2-6. Compound 1 solvate will be started on Day 2 of cycle 1 at a dose of 420 mg Compound 1 (3×140-mg capsules) orally daily and will be continued daily.

Primary Outcome Measures: Progression free survival (PFS) [Time Frame: 3 months]—progression free survival defined as the time interval from treatment to progressive disease or death, whichever happens earlier. Patients in complete remission (CR), partial remission (PR) or stable disease (SD) are all counted as progression-free. Survival or times to progression functions estimated using the Kaplan-Meier method.

Secondary Outcome Measures: Toxicity [Time Frame: 3 months]—toxicity reported by type, frequency and severity. Worst toxicity grades per patient tabulated for selected adverse events and laboratory measurements. Toxicity (grade 3 or 4) monitored based on the Bayesian model (beta-binomial) by assuming a priori probability of toxicity following beta(1,1).

Eligibility: 18 Years and older; both genders are eligible.

Inclusion Criteria: 1. Patients must have a diagnosis of high-risk CLL/SLL and be previously treated with up to 3 lines of prior therapy. High-risk CLL and high-risk SLL is defined by the presence of a 17p deletion or 11q deletion or TP53 mutation. Any CLL and SLL patient who has a short remission duration of less than 3 years after prior first-line chemo-immunotherapy, such as the FCR regimen, also fulfills criteria of high-risk CLL/SLL, regardless of the presence or absence of cytogenetic abnormalities. 2. CLL and SLL patients with 17p deletion or TP53 mutation will not be required to have received any prior therapy, given the poor outcome of CLL/SLL patients to standard frontline chemo-immunotherapy, such patients will be eligible if they are untreated or if they have received up to 3 lines of prior therapy. 3. Patients must have an indication for treatment by 2008 IWCLL Criteria. 4. Patients age >18 years at the time of signing informed consent. Understand and voluntarily sign an informed consent. Be able to comply with study procedures and follow-up examinations. 5. ECOG/WHO performance status of 0-1. 6. Patients of childbearing potential must be willing to practice highly effective birth control (e.g., condoms, implants, injectables, combined oral contraceptives, some intrauterine devices [IUDs], sexual abstinence, or sterilized partner) during the study and for 30 days after the last dose of study drug. Women of childbearing potential include any female who has experienced menarche and who has not undergone successful surgical sterilization (hysterectomy, bilateral tubal ligation, or bilateral oophorectomy) or is not postmenopausal. Post menopause is defined as follows: Amenorrhea >/=12 consecutive months without another cause and a documented serum follicle stimulating hormone (FSH) level >35 mIU/mL; a male of childbearing potential is any male that has not been surgically sterilized. 7. Adequate renal and hepatic function as indicated by all of the following: Total bilirubin </=1.5×institutional Upper Limit of Normal (ULN) except for patients with bilirubin elevation due to Gilbert's disease who will be allowed to participate; an ALT </=2.5×ULN; and an estimated creatinine clearance (CrCl) of >30 mL/min, as calculated by the Cockroft-Gault equation unless disease related. 8. Free of prior malignancies for 3 years with exception of currently treated basal cell, squamous cell carcinoma of the skin, or carcinoma in situ of the cervix or breast. 9. A urine pregnancy test (within 7 days of Day 1) is required for women with childbearing potential

Exclusion Criteria: 1. Pregnant or breast-feeding females. 2. Treatment including chemotherapy, chemo-immunotherapy, monoclonal antibody therapy, radiotherapy, high-dose corticosteroid therapy (more than 60 mg Prednisone or equivalent daily), or immunotherapy within 21 days prior to enrollment or concurrent with this trial. 3.Investigational agent received within 30 days prior to the first dose of study drug or have previously taken Compound 1 solvate. If received any investigational agent prior to this time point, drug-related toxicities must have recovered to Grade 1 or less prior to first dose of study drug. 4. Systemic fungal, bacterial, viral, or other infection not controlled (defined as exhibiting ongoing signs/symptoms related to the infection and without improvement, despite appropriate antibiotics or other treatment). 5. Patients with uncontrolled Autoimmune Hemolytic Anemia (AIHA) or autoimmune thrombocytopenia (ITP). 6. Patients with severe hematopoietic insufficiency, as defined by an absolute neutrophil count of less than 500/micro-L and/or a platelet count of less than 30,000/micro-L at time of screening for this protocol. 7. Any other severe concurrent disease, or have a history of serious organ dysfunction or disease involving the heart, kidney, liver or other organ system that may place the patient at undue risk to undergo therapy with Compound 1 solvate and rituximab. 8. Significant cardiovascular disease such as uncontrolled or symptomatic arrhythmias, congestive heart failure, or myocardial infarction within 6 months of screening, or any Class 3 or 4 cardiac disease as defined by the New York Heart Association Functional Classification. 9. Significant screening ECG abnormalities including left bundle branch block, 2nd degree AV block type II, 3rd degree block, bradycardia, and QTc >470 msec. 10. Any serious medical condition, laboratory abnormality, or psychiatric illness that places the subject at unacceptable risk if he/she were to participate in the study. 11. History of stroke or cerebral hemorrhage within 6 months. 12. Evidence of bleeding diathesis or coagulopathy. 13. Major surgical procedure, open biopsy, or significant traumatic injury within 28 days prior to Day 1, anticipation of need for major surgical procedure during the course of the study. 14. Minor surgical procedures, fine needle aspirations or core biopsies within 7 days prior to Day 1. Bone marrow aspiration and/or biopsy are allowed. 15. Serious, non-healing wound, ulcer, or bone fracture. 16. Treatment with Coumadin. Patients who recently received Coumadin must be off Coumadin for at least 7 days prior to start of the study. 17. Any chemotherapy (e.g., bendamustine, cyclophosphamide, pentostatin, or fludarabine), immunotherapy (e.g., alemtuzumab, or ofatumumab), bone marrow transplant, experimental therapy, or radiotherapy is prohibited during therapy on this study. 18. Use of medications known to prolong QTc interval or that may be associated with Torsades de Pointes are prohibited within 7 days of starting study drug and during study-drug treatment.

The examples and embodiments described herein are illustrative and various modifications or changes suggested to persons skilled in the art are to be included within this disclosure. As will be appreciated by those skilled in the art, the specific components listed in the above examples may be replaced with other functionally equivalent components, e.g., diluents, binders, lubricants, fillers, and the like.

1. A solvate of 1-((R)-3-(4-amino-3-(4-phenoxyphenyl)-1H-pyrazolo[3,4-d]pyrimidin-1-yl)piperidin-1-yl)prop-2-en-1-one, wherein 1-((R)-3-(4-amino-3-(4-phenoxyphenyl)-1H-pyrazolo[3,4-d]pyrimidin-1-yl)piperidin-1-yl)prop-2-en-1-one is solvated with acetophenone or benzyl acetate.

2. The solvate of claim 1, which is in a crystalline form.

3-30. (canceled)

31. The crystalline form of claim 2, wherein the solvate is an acetophenone solvate that has at least one of the following properties:
(a) an X-ray powder diffraction (XRPD) pattern substantially similar to the one set forth in FIG. 9;
(b) an XRPD pattern comprising at least two, at least four, at least six, at least eight, at least ten, or twelve peaks at about 7.6° 2-Theta, about 8.8° 2-Theta, about 15.2° 2-Theta, about 17.6° 2-Theta, about 18.9° 2-Theta, about 19.5° 2-Theta, about 20.4° 2-Theta, about 21.0° 2-Theta, about 21.3° 2-Theta, about 21.8° 2-Theta, about 24.3° 2-Theta, and about 24.8° 2-Theta;
(c) a differential scanning calorimetry (DSC) thermogram substantially similar to the one set forth in FIG. 10;
(d) a DSC thermogram having an endotherm with a peak at about 96° C.;
(e) thermo-gravimetric analysis (TGA) thermogram substantially similar to the one set forth in FIG. 10;
(f) unit cell parameters approximately equal to the following at a temperature of approximately 100(2) K:

| Molecular formula | C₃₃H₃₂N₆O₃ | | | | |
|---|---|---|---|---|---|
| Molecular weight | 560.64 | | | | |
| Crystal system | Triclinic | | | | |
| Space group | P1 | a | 11.3552(5) Å | α | 79.657(3)° |
| | | b | 11.7741(4) Å | β | 70.352(4)° |
| | | c | 12.2064(4) Å | γ | 67.080(4)° |
| | |
|---|---|
| V | 1413.38(11) Å³ |
| Z | 2 |
| Density (calculated) | 1.317 Mg/m³ |
| Absorption coefficient | 0.699 mm⁻¹ |
| Wavelength | 1.54178 Å |
| F(000) | 592 |
| T | 100(2) K |

or
(g) combinations thereof.

32. The crystalline form of claim 31, wherein the crystalline form has an XRPD pattern substantially similar to the one set forth in FIG. 9.

33. The crystalline form of claim 31, wherein the crystalline form has an XRPD pattern comprising at least two peaks at about 7.6° 2-Theta, about 8.8° 2-Theta, about 15.2° 2-Theta, about 17.6° 2-Theta, about 18.9° 2-Theta, about 19.5° 2-Theta, about 20.4° 2-Theta, about 21.0° 2-Theta, about 21.3° 2-Theta, about 21.8° 2-Theta, about 24.3° 2-Theta, and about 24.8° 2-Theta.

34. The crystalline form of claim 31, wherein the DSC thermogram has an endotherm with a peak at about 96° C.

35. The crystalline form of claim 31, wherein the crystalline form has a DSC thermogram substantially similar to as the one set forth in FIG. 10.

36-46. (canceled)

47. The crystalline form of claim 2, wherein the solvate is an acetophenone solvate that has at least one of the following properties:
(a) an XRPD pattern substantially similar to the one set forth in FIG. 13;
(b) an XRPD pattern comprising at least two, at least four, at least six, at least eight, or nine peaks at about 6.5° 2-Theta, about 13.0° 2-Theta, about 17.6° 2-Theta, about 18.4° 2-Theta, about 19.9° 2-Theta, about 21.0° 2-Theta, about 21.5° 2-Theta, about 22.1° 2-Theta, and about 23.9° 2-Theta;
(c) a DSC thermogram substantially similar to the one set forth in FIG. 14;
(d) a DSC thermogram having an endotherm with a peak at about 127° C.;
(e) thermo-gravimetric analysis (TGA) thermogram substantially similar to the one set forth in FIG. 14;
or
(f) combinations thereof.

48. The crystalline form of claim 47, wherein the crystalline form has an XRPD pattern substantially similar to the one set forth in FIG. 13.

49. The crystalline form of claim 47, wherein the crystalline form has an XRPD pattern comprising at least two peaks at about 6.5° 2-Theta, about 13.0° 2-Theta, about 17.6° 2-Theta, about 18.4° 2-Theta, about 19.9° 2-Theta, about 21.0° 2-Theta, about 21.5° 2-Theta, about 22.1° 2-Theta, and about 23.9° 2-Theta.

50. The crystalline form of claim 47, wherein the DSC thermogram has an endotherm with a peak at about 127° C.

51. The crystalline form of claim 47, wherein the crystalline form has a DSC thermogram substantially similar to the one set forth in FIG. 14.

52. The crystalline form of claim 47, wherein the crystalline form has a TGA thermogram substantially similar to the one set forth in FIG. 14.

53-61. (canceled)

62. The crystalline form of claim 2, wherein the solvate is a benzyl acetate solvate that has at least one of the following properties:
(a) an XRPD pattern substantially similar to the one set forth in FIG. 17;
(b) an XRPD pattern comprising at least two, at least four, at least six, or eight peaks at about 12.8° 2-Theta, about 17.8° 2-Theta, about 18.7° 2-Theta, about 19.2° 2-Theta, about 20.1° 2-Theta, about 20.7° 2-Theta, about 22.1° 2-Theta and about 22.9° 2-Theta;
(c) a DSC thermogram substantially similar to the one set forth in FIG. 18;
(d) a DSC thermogram having an endotherm with a peak at about 158° C.;
(e) a thermo-gravimetric analysis (TGA) thermogram substantially similar to the one set forth in FIG. 18;
or
(f) combinations thereof.

63. The crystalline form of claim 62, wherein the crystalline form has an XRPD pattern substantially the same as shown in FIG. 17.

64. The crystalline form of claim 62, wherein the crystalline form has an XRPD pattern comprising at least two peaks at about 12.8° 2-Theta, about 17.8° 2-Theta, about 18.7° 2-Theta, about 19.2° 2-Theta, about 20.1° 2-Theta, about 20.7° 2-Theta, about 22.1° 2-Theta and about 22.9° 2-Theta.

65-67. (canceled)

68. A pharmaceutical composition comprising a crystalline form of claim 31, and at least one pharmaceutically acceptable ingredient.

69-73. (canceled)

74. A pharmaceutical composition comprising the crystalline form of claim 47, and at least one pharmaceutically acceptable ingredient.

75. The crystalline form of claim 62, wherein the DSC thermogram has an endotherm with a peak at about 158° C.

76. A pharmaceutical composition comprising the crystalline form of claim 62, and at least one pharmaceutically acceptable ingredient.
